(19) United States
(12) Patent Application Publication
(10) Pub. No.: US 2018/0110837 A1
(43) Pub. Date: Apr. 26, 2018

(54) METHODS AND ASSAYS RELATING TO MACROPHAGE DIFFERENTIATION

(71) Applicant: THE BRIGHAM AND WOMEN'S HOSPITAL, INC., Boston, MA (US)

(72) Inventor: Ali HAFEZI-MOGHADAM, Jamaica Plain, MA (US)

(73) Assignee: THE BRIGHAM AND WOMEN'S HOSPITAL, INC., Boston, MA (US)

(21) Appl. No.: 15/621,062

(22) Filed: Jun. 13, 2017

Related U.S. Application Data
(63) Continuation of application No. 14/649,755, filed on Jun. 4, 2015, now abandoned.
(60) Provisional application No. 61/737,462, filed on Dec. 14, 2012.

(51) Int. Cl. A61K 38/21; A61K 38/20; A61K 31/513; A61K 31/551; G01N 33/569; C12N 5/0786; A61K 31/435; A61K 38/19; A61K 35/12; A61K 39/00

(52) U.S. Cl. CPC A61K 38/217; A61K 38/2066; A61K 31/513; A61K 31/551; G01N 33/56972; C12N 5/0645; G01N 2800/7014; A61K 31/435; A61K 38/2026; A61K 38/19; A61K 35/12; C12N 2501/727; A61K 2039/57; A61K 38/2086

(57) ABSTRACT

The technology described herein is directed to methods and assay related to macrophage differentiation and the role of such in pathogenesis.

### CROSS-REFERENCE TO RELATED APPLICATIONS

This application is a continuation of U.S. application Ser. No. 14/649,755 filed Jun. 4, 2015, which is a 35 U.S.C. § 371 National Phase Entry Application of International Application No. PCT/US2013/074922 filed Dec. 13, 2013, which designates the U.S., and which claims benefit under 35 U.S.C. § 119(e) of U.S. Provisional Application No. 61/737,462 filed Dec. 14, 2012, the contents of which are incorporated herein by reference in their entirety.

### GOVERNMENT SUPPORT

This invention was made with government support under Grant Nos. 1R01DK083567 and A1050775 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Jun. 13, 2017, is named 043214-076601-PCT_SL.txt and is 235,515 bytes in size.

### TECHNICAL FIELD

The technology described herein relates to macrophage differentiation.

### BACKGROUND

Age-related macular degeneration (AMD) is the leading cause of adult vision loss. In many cases, AMD can have a proliferative nature due to pathogenic angiogenesis called choroidal neovascularization (CNV). Why this angiogenic process is activated in the eyes of affected subjects is not currently known.

Vascular endothelial growth factor A (VEGF-A) has long been implicated in angiogenesis, but is also important for normal functions in the eye. Current drug therapy in AMD is based on repeated intravitreal injections of VEGF-A inhibitors (Martin et al. NEJM 2011 364:1897-1908), which is not free of risks to the retina (Nishijima, K. et al. Am J Pathol 2007 171, 53-67 and Maharaj, A. S. et al. J Exp Med 2008 205, 491-501). A multi-center cohort study showed macular atrophy in virtually all long-term treated AMD cases, one third of which suffered an alarming visual decay (Rofagha, S., et al. Ophthalmology, 2013).

### SUMMARY

Mature macrophages can have either an M1 phenotype (pro-inflammatory) or an M2 phenotype (anti-inflammtory). As described herein, the inventors have discovered that the amount of M2-type macrophages increases with age and in eyes affected by AMD, particularly those experiencing CNV. Decreasing the number of M2 macrophages decreases the amount of pathogenic angiogenesis and reduces damage to the eye.

It is further demonstrated herein that this shift towards the M2 phenotype is promoted by ROCK2, but not ROCK1. Thus, the inhibiting ROCK2 decreases both the amount of M2-type macrophages and pathogenic angiogenesis. The work presented herein describes a previously unknown molecular switch for macrophage polarization through the ROCK signaling pathway.

In one aspect, described herein is a method of treating a condition selected from the group consisting of pathogenic angiogenesis; vascular leakage; and aging or age-related conditions; the method comprising administering a M2 or MaDAM macrophage inhibitor to a subject. In one aspect, described herein is a method of treatment comprising administering a M2 or MaDAM macrophage inhibitor to a patient determined to have an increased level of M2 or MaDAM cells in a tissue.

In some embodiments, the M2 or MaDAM macrophage is a CD11b(+) cell. In some embodiments, the M2 or MaDAM macrophage is a CD163(+) cell. In some embodiments, the M2 or MaDAM macrophage is a CD206(+) cell. In some embodiments, the pathogenic angiogenesis is associated with a condition selected from the group consisting of AMD, CNV, or aging. In some embodiments, the vascular leakage is associated with AMD. In some embodiments, the M2 macrophage inhibitor is a pan-ROCK inhibitor. In some embodiments, the pan-ROCK inhibitor is selected from the group consisting of Fasudil; HP1152P; and Y-27632. In some embodiments, the M2 macrophage inhibitor is a ROCK2-specific inhibitor. In some embodiments, the ROCK2 inhibitor does not inhibit ROCK1. In some embodiments, the ROCK2 inhibitor does not affect the cytoskeleton. In some embodiments, the ROCK2 inhibitor does not reduce recruitment. In some embodiments, the ROCK2 inhibitor is SLx2119. In some embodiments, the M2 macrophage inhibitor is a M1-promoting cytokine. In some embodiments, the M1-promoting cytokine is selected from the group consisting of INF-γ and LPS. In some embodiments, the inhibitor is not a direct modulator of VEGF-A.

In one aspect, described herein is a method of treating a condition selected from the group consisting of pathogenic angiogenesis; vascular leakage; and aging or age-related conditions the method comprising administering a M1 macrophage to a subject. In some embodiments, the pathogenic angiogenesis is associated with a condition selected from the group consisting of AMD, CNV, or aging. In some embodiments, the pathogenic vascular leakage is associated with AMD. In some embodiments, the M1 macrophage is administered via intravitreal injection.

In one aspect, described herein is a method of promoting macrophage differentiation to the M1 phenotype, the method comprising contacting a macrophage with M2 macrophage inhibitor. In some embodiments, the M2 macrophage inhibitor is a ROCK2-specific inhibitor. In some embodiments, the ROCK2 inhibitor does not inhibit ROCK1. In some embodiments, the ROCK2 inhibitor does not affect the cytoskeleton. In some embodiments, the ROCK2 inhibitor does not reduce recruitment. In some embodiments, the ROCK2 inhibitor is SLx2119. In some embodiments, the M2 macrophage inhibitor is a M1-promoting cytokine. In some embodiments, the M1-promoting cytokine is selected from the group consisting of INF-γ and LPS. In some embodiments, the inhibitor is not a direct modulator of VEGF-A.

In one aspect, described herein is a method of treating an inflammatory or autoimmune disease the method comprising administering a M1 macrophage inhibitor to a subject. In some embodiments, the M1 macrophage inhibitor is a ROCK1 inhibitor. In some embodiments, the ROCK1 inhibitor is a ROCK1-specific inhibitor. In some embodiments, the ROCK1 inhibitor does not inhibit ROCK2. In some embodiments, the ROCK1 inhibitor does affect the cytoskeleton. In some embodiments, the ROCK1 inhibitor reduces recruitment. In some embodiments, the ROCK1 inhibitor is selected from the group consisting of GSK 429286; a dihydropyrimidinone; and a dihydropyrimidine. In some embodiments, the M1 macrophage inhibitor is a M2-promoting cytokine. In some embodiments, the M2-promoting cytokine is selected from the group consisting of IL-4; IL-10; and IL-13. In some embodiments, the inhibitor is not a direct modulator of VEGF-A.

In one aspect, described herein is a method of promoting macrophage differentiation to the M2 phenotype, the method comprising contacting a macrophage with a M1 macrophage inhibitor. In some embodiments, the M1 macrophage inhibitor is a ROCK1 inhibitor. In some embodiments, the ROCK1 inhibitor is a ROCK1-specific inhibitor. In some embodiments, the ROCK1 inhibitor does not inhibit ROCK2. In some embodiments, the ROCK1 inhibitor does affect the cytoskeleton. In some embodiments, the ROCK1 inhibitor reduces recruitment. In some embodiments, the ROCK1 inhibitor is selected from the group consisting of GSK 429286; a dihydropyrimidinone; and a dihydropyrimidine. In some embodiments, the M1 macrophage inhibitor is a M2-promoting cytokine. In some embodiments, the M2-promoting cytokine is selected from the group consisting of IL-4; IL-10; and IL-13. In some embodiments, the inhibitor is not a direct modulator of VEGF-A.

In one aspect, described herein is a method of determining if a tissue is affected by pathogenic angiogenesis or vascular leakage, the method comprising measuring the level of M2 or MaDAM cells present in the tissue; and determining the tissue is affected by pathogenic angiogenesis if the level of M2 or MaDAM cells is increased relative to a control. In some embodiments, the pathogenic angiogenesis or vascular leakage is associated with AMD or CNV. In some embodiments, the level of M2 or MaDAM cells is determined by measuring the level of CD11b. In some embodiments, the level of M2 or MaDAM cells is determined by measuring the level of CD163. In some embodiments, the level of M2 or MaDAM cells is determined by measuring the level of CD206. In some embodiments, the level of M2 or MaDAM cells is determined by measuring the level of ROCK1 or ROCK2. In some embodiments, the level of M2 or MaDAM cells is determined by measuring the level of a marker selected from the group consisting of arginase 1; YM 1; Fizz1; CCL5; IL-10; CCL3; MYPT1; I×Ba; NF-κB; IL-4; CCR3; MLC; RhoA; and iNOS. In some embodiments, the method further comprises a step of administering a treatment for pathogenic angiogenesis if the level of M2 or MaDAM cells is increased relative to the control. In some embodiments, the treatment comprises the administration of an M2 macrophage inhibitor and/or M1 macrophage as described herein.

### BRIEF DESCRIPTION OF THE DRAWINGS

FIGS. 1A-1F demonstrate that CNV depends on ROCK signaling. ROCK signaling was quantified in CNV and the effect of ROCK inhibition was studied in vivo. FIG. 1A depicts the results of Western blot analysis with anti-pMYPT1, anti-MYPT1, anti-ROCK1, anti-ROCK2 or anti-β-tubulin Abs, using whole cell lysates from choroids of normal and CNV eyes at the indicated time points after laser injury. Vehicle and inhibitor treatment of day 7 CNV tissues were screened for anti-pMYPT1, anti-MYPT1, anti-ROCK1, anti-ROCK2, as well as (FIG. 1B) anti-RhoA, anti-RhoE, and anti-pMLC Abs, n=3. (FIG. 1C depicts the quantification of mouse CNV lesions treated with vehicle, fasudil (n=5, 40 lesions), ROCK2 inhibitor at different concentrations (n=4, 32 lesions in each group) and in ROCK1+/−TieCre mouse (n=5, 40 lesions). FIG. 1D depicts quantification of leakage from the angiogenic vessels visualized by fluorescein angiography and quantified in early (1-2 min) and late-phase (6-8 min) angiograms of Brown Norway rats treated with vehicle, fasudil or ROCK2 inhibitor, as well as (FIG. 1E) in ROCK1+/−TieCre mouse. FIG. 1F depicts the results of fluorescent angiography in monkey and CNV thickness measurements with vehicle or fasudil treatment (n=4). The percentage of lesions graded as 0, I, IIa, defined as no to moderate leakage, and Hb, clinically relevant leakage (n=4, 32 lesions). *P<0.05; **P<0.01.

FIGS. 2A-2D demonstrate that lack of endothelial NFκ-B signaling does not affect CNV. FIG. 2A depicts Western blot analysis of pIκB-α, IκB-α, pNF-κB p65, NF-κB, and β-tubulin in whole cell lysates of lasered eye extracts at different time points after laser injury. FIG. 2B depicts a representative western blot of anti-pIκB-α, anti-IκB-α, anti-pNF-κB p65, anti-NF-κB, anti-GAPDH with fasudil (20 mg/kg), Y-27632 (10 mg/kg) or ROCK2 inhibitor (10 mg/kg) treatment in choroids with CNV (day 7) with the corresponding quantifications. FIG. 2C depicts quantitative analysis of CNV volume (n=5, 40 lesions). *P<0.05; **P<0.01. FIG. 2D depicts quantification of Representative early phase (1-2 min) and late-phase (6-8 min) fluorescein angiograms in C57BL/6 and Tie-1-ΔN mice.

FIGS. 3A-3C demonstrate ROCK-mediated regulation of inflammatory leukocyte infiltration during CNV. FIG. 3A depicts quantification of the number of F4/80-positive macrophages in CNV lesions in C57BL/6 mice treated with vehicle, fasudil or ROCK2 inhibitor, or in CD18−/− mice (n=3). FIG. 3B depicts the impact of fasudil on macrophage infiltration in CNV of monkey (n=4). The graph depicts quantification of the number of CD68-positive leukocytes in CNV lesions (n=4) of CNV lesions, in lasered monkeys treated with vehicle or fasudil. **P<0.01. Scale bar, 100 μm. FIG. 3C depicts quantification of Ex vivo imaging of impact of ROCK inhibitors on MCP-1-mediated leukocyte transmigration. AO(+) leukocytes and Con A(+) angiogenic vessels in MCP-1-implanted corneas, 2 h after AO injection, 24 hours after pellet implantation with vehicle, fasudil, Y-27632 or ROCK2 inhibitor treatment. Graph depicts quantification of the number of AO(+) leukocytes in areas of MCP-1-implanted corneas 2 h after AO injection, 24 hours after pellet implantation. n=4; *P<0.05, **P<0.01.

FIGS. 4A-4C demonstrate the polarization of ocular infiltrating macrophages by ROCK. FIG. 4A depicts Western blot analysis with anti-IL-4, anti-CD163, anti-CCR3, anti-CCR7, anti-CD80 or anti-β-tubulin Abs, using whole cell lysates from lasered mouse choroids at the indicated time points after laser injury. FIG. 4B depicts FACS analysis of infiltrating cells from untreated and laser-injured choroids at the different time points after laser injury. Cells were stained for CD11b, CD80, and CD206. The percentages of infiltrating CD11b(+)CD206(+), CD11b(+)CD80(+), CD11b(−) CD206(+), CD11b(−)CD80(+) cells in CNV at the indicated times (n=6). FIG. 4C depicts FACS analysis of infiltrating cells from CNV eyes (choroid) with vehicle, fasudil or ROCK2 inhibitor treatment at day 7 with PE-CD11b mAb and FITC-CD206 mAb. The percentages of infiltrating CD11b(+)CD206(+) cells and CD11b(+)CD206(−) cells in CNV with the treatment at day 7 (n=6). *P<0.05; **P<0.01.

FIG. 5 demonstrates that isoform-specific inhibition of ROCK determines macrophage fate. ROCK isoforms were blocked in RAW 264.7, U937 and BMDM cells in M0-, M1-, or M2 environments. Chemokines, cell markers, and genes were quantified by luminex, flow cytometry, or rtPCR. Data represent average from three or more independent experiments. RT-PCR data was evaluated after subtraction of the specific endogenous control gene expression (18S rRNA). *P<0.05; **P<0.01

FIGS. 6A-6G demonstrate that age-induced ROCK-signaling and M2 differentiation cause CNV. FIG. 6A dempicts CNV results from mice after intravitreal injections of Vehicle, M0, M1- with or without ROCK2 inhibitor or M2-differentiated macrophages. FIG. 6B depicts quantification of CNV lesions from mice after intravitreal cytokine injections, M1 cocktail: INF-γ and LPS, M2 cocktail: IL-4, Il-10, IL13. FIG. 6C depicts leakage from the angiogenic vessels was visualized by fluorescein angiography and quantified in early (1-2 min) and late-phase (6-8 min) angiograms of mice injected with either M1 or M2 cocktail. Scale bar, 1 mm. FIG. 6D depicts the quantification of CNV lesions from C57BL mice, IL-12−/− and Adiponectin−/− mice with and without ROCK2 inhibitor. FIG. 6E depicts representative western blot of normal and CNV (day 3) choroids from young (8-12 week old) and aged (>16 month old) WT mice and quantifications; n=3, *P<0.05; **P<0.01. FIG. 6F depicts Western blot of normal choroids from young (8-12 week old) and aged (>16 month old) MCP-1−/− mice, and quantifications; n=3, *P<0.05; **P<0.01. FIG. 6G depicts representative western blot of anti-IL-4, anti-CD80 and anti-CCR7 with fasudil (20 mg/kg), Y-27632 (10 mg/kg) or ROCK2 inhibitor (10 mg/kg) treatments in choroids with CNV (day 7) with the corresponding quantifications.

FIGS. 7A-7B demonstrate characterization of the ROCK2 inhibitor. The IC50 of the ROCK2 inhibitor, SLx-2119, and the pan ROCK inhibitor, Y-27632, for ROCK1 and ROCK2 were previously published (WO2010104851A1, FIG. 10, sheet 18). SLx-2119 selectively inhibited ROCK2 (IC50˜105 nM). FIG. 7A demonstrates that in contrast to Y-27632 (IC50 111 nM), SLx-2119 did not affect ROCK1 enzymatic activity at concentrations up to 10 μM (IC50 24 μM). Briefly, recombinant ROCK1 and ROCK2 enzymes containing the truncated catalytic domains (PV3691 and PV3759) were purchased from Invitrogen and enzymatic activity was determined using [γ33P]ATP (5 μM) and S6 kinase substrate (17 μM). The reaction was run for 45 min at room temperature and was terminated by the addition of phosphoric acid. [γ33P] phosphorylated S6 peptide was isolated by membrane filtration. The background was determined by running the reaction without enzyme. Radioactivity was assessed using a Microbeta Jet. Schueller et al. reported a >100 fold higher selectivity of the inhibitor against ROCK2 (IC50=125±25 nM) versus ROCK1 (IC50=13±1 μM) 56. A subsequent study, using a cell free radiometric enzyme assay showed a selective inhibition of human ROCK2 (IC50=105 nM), while inhibition of ROCK1 was minimal (IC50=24 04) 57. FIG. 7B depicts the comparative enzyme inhibitor kinetics (Ki) of two existing pan ROCK inhibitors, fasudil and Y-27632 58, and the selective ROCK2 inhibitor, SLx-2119. SLx2119 potently inhibits ROCK2, without affecting ROCK1. In comparison, Y-27632 and fasudil, showed equipotent inhibitory activities against ROCK1 and ROCK2. Therefore, in this study, the biological functions of the ROCK2 inhibitor were compared with the pan ROCK inhibitors fasudil and Y-27632. IC50 was determined by curve fitting using GraphPad Prizm software (available on the world wide web at www.graphpad.com). The sigmoidal dose-response (variable slope) equation type analysis was used to generate the IC50 values. Ki values were calculated using the Cheng Prusoff equation of Ki=IC50/(1+[S]/Km), where [S] and Km are the concentration of ATP and the Km value of ATP, respectively 59. Fasudil (HA-1077, 5-(1,4-diazepan-1-ylsulfonyl)isoquinoline hydrochloride, C14H17N3O2S.HCl, molecular weight 327.83) is in some countries an approved drug for the human use. In addition to ROCK, fasudil also inhibits, PKA, PKG, PKC, and MLCK with Ki values of 0.33 μM, 1.6 μM, 1.6 μM, 3.3 μM and 36 μM, respectively. Recently, Anastassiadis et al. determined the gini score, a measure of statistical dispersion, for over 175 inhibitors 60. The gini score reflects, on a scale of 0 to 1, the degree to which the aggregate inhibitory activity of a compound (calculated as the sum of inhibition for all kinases) is directed toward only a single target (a gini score of 1 or highest possible selectivity) or is distributed equally across all tested kinases (a gini score of 0 or promiscuous inhibition). The gini range of all examined molecules was 0.20 and 0.81. Fasudil scored 0.63 and ranked 74th, while over 100 inhibitors showed a more favorable gini score 60. Next to inhibiting both ROCK isoforms, fasudil and Y-27632 also affect protein kinase A and C 61. While the same numeric value of gini score may result from different inhibitory distributions, fasudil's inhibitory score of 0.63 suggests the need for development of more selective molecules.

FIG. 8 demonstrates CNV reduction with preventive and therapeutic ROCK inhibition. FIG. 8 depicts quantitative analysis of CNV volume of CNV lesions (day 14) in choroidal flat mounts from mice treated with vehicle, fasudil or ROCK2 inhibitor from day 1 or day 7 after laser injury. (n=4, 32 lesions).

FIG. 9 demonstrates that intravitreal ROCK inhibition reduces CNV. FIG. 9 depicts quantitative analysis of CNV volume of CNV lesions (day 7) in the choroidal flat mounts from rats treated with vehicle, fasudil or ROCK2 inhibitor intravitreously. (n=4, 32 lesions).

FIG. 10 demonstrates the lack of abnormality in electroretinogram (ERG) with ROCK inhibition. FIG. 10 depicts ERG of rats with or without intravitreal injections of vehicle, fasudil, or ROCK2 inhibitor on days 0, 3, and 6 after treatment. ROCK inhibition at concentrations that reduce CNV does not cause functional ERG changes.

FIGS. 11A-11B demonstrate the impact of ROCK inhibition on cytoskeletal structures. FIG. 11A depicts HUVEC cells stained for the cytoskeletal proteins F-actin and paxillin. ROCK1 blockade diminishes but ROCK2 blockade does not affect cytoskeletal structures. Scale bar, 20 μm. FIG. 11B depicts Western analysis of HUVECs transfected with siRNA targeting of ROCK1, ROCK2, or both. Negative control, a scramble 20-mer RNA. BlockIt (Invitrogen), a fluorescently labeled RNA to track uptake/transfection efficiency.

FIG. 12 depicts a time course of CD11b(+)CD206(−) cell accumulation in CNV. FACS analysis shows the initial increase of the infiltrated CD11b(+)CD206(−) cells in CNV (n=6). The initial peak on day 1 could in part be attributed to neutrophils. *P<0.05, **P<0.01.

FIGS. 13A-13B demonstrate ROCK signaling in macrophages is distinct from VEGF expression. VEGF is an important growth factor in AMD pathology. VEGF-A inhibition is the current standard in AMD treatment. To examine the role of ROCK signaling in VEGF-A secretion in macrophages, this growth factor was measured in cultured RAW 264.7 monocytes that were in M0, M1, and M2 state of differentiation. Neither pharmacologic (FIG. 13A) nor (FIG. 13B) gene blockade of ROCK isoforms affected VEGF-A gene or protein expression in M0, M1, or M2 macrophages. These data indicate that the anti-angiogenic and anti-leakage properties of ROCK inhibition is unrelated to VEGF-A secretion from macrophages.

FIG. 14 demonstrates macrophage polarization. For quality control, the M1 and M2 differentiated bone marrow derived macrophages were characterized by flow cytometry.

FIG. 15 demonstrates CCR3 expression in CNV. CCR3 is a chemokine receptor involved in eosinophils and basophils trafficking. CCR3 is also found in TH2 cells 62. It is thought to take part in the type 2 response 13. Monocytes also express CCR3, albeit at lower levels. CCR3 expression is increased in CD14(+) monocytes in patients with rheumatoid or other types of arthritis, indicating the variable expression of this molecule in pathologic conditions 63. For instance, CCR3 on macrophages is dramatically upregulated through HIV-1 tat protein 64. CCR3 was reported to be elevated in CNV without any involvement of inflammation 31. Surprisingly, these results did not show a difference in CCR3 in CNV compared to unlasered controls.

FIG. 16 demonstrates Age-dependent ROCK signaling in MCP-1−/− mice. Depicted is the quantification of western blot results from normal choroids of young (8-12 week old) and aged (>16 month old) MCP-1−/− mice. CCR3, ROCK1, ROCK2, IκB-α, CCR7, and CD80. n=3. The M-1 specific markers CCR7 and CD80 remained unchanged between young and aged animals.

### DETAILED DESCRIPTION

As described herein, the inventors have discovered that the balance of two ROCK isoforms, ROCK1 and ROCK2 controls macrophage differentiation and cell fate. Modulation of the balance of ROCK1/ROCK2 can be used, e.g. to control macrophage development and treat pathogenic conditions characterized by macrophage imbalance, e.g., age-related macular degeneration (AMD).

As used herein, “ROCK” or “Rho-associated, coiled-coil-containing protein kinase” refers to a kinase involved in cytoskeletal rearrangement, contractility, angiogenesis, and inflammation. Upstream of ROCK are RhoA and RhoE which are involved in cytoskeletal functions. Immediate ROCK substrates are myosin light chain (MLC), myosin binding subunit of myosin phosphatase (MYPT), and ezrin/radixin/moesin (ERM) proteins, while downstream targets include IκB-α and NF-κB. ROCK phosphorylates MLC phosphatase, causing smooth muscle contraction and vasoconstriction. Two forms of ROCK exist, i.e., ROCK1 and ROCK2. As used herein, “ROCK1” refers to a human ROCK1 or a homolog or variant thereof (NCBI Gene ID: 6093; SEQ ID NOs: 1-2). As used herein, “ROCK2” refers to human ROCK2 or a homolog or variant thereof (NCBI Gene ID: 9475; SEQ ID NOs: 3-4).

The balance of the two isoforms of ROCK pushes an undifferentiated macrophage towards a particular differentiation fate. An undifferentiated M0 macrophage can polarize into either a pro-inflammatory M1 macrophage or an anti-inflammatory M2 macrophage. An M1 macrophage can be characterized by the expression of CCL3, CCL5, CD80, CCR7, iNOS and INF-γ. An M2 macrophage can be characterized by the expression of CCL22, CD206, CD163, YM1, Fizz1, and arginase 1. Described herein is a previously unknown subset of M2 macrophages termed Macular Degeneration Associated Macrophages (MaDAMs). MaDAMs can be distinguished by their location (e.g., their presence in AMD tissues). MaDAMs express both ROCK isoforms.

Higher levels of ROCK2 expression and/or activity induces differentions towards the M2 phenotype, while lower levels of ROCK2 expression and/or activity induces differentiation towards the M1 phenotype. A surfeit of either type of macrophage, as described herein, can lead to pathogenic perturbations of a subject's physiology. Thus, the ability to modulate macrophage differentiation, in accordance with the methods described herein, can permit treatment of certain diseases and conditions.

In one aspect, described herein is a method of promoting macrophage differentiation to the M1 phenotype, the method comprising contacting a macrophage with a M2 macrophage inhibitor. As used herein, an “inhibitor” is refers to an agent which can decrease the level, expression and/or activity of the target (e.g., the level of a M2 macrophage inhibitor, or ROCK2), e.g. by at least 10% or more, e.g. by 10% or more, 50% or more, 70% or more, 80% or more, 90% or more, 95% or more, or 98% or more. The efficacy of an inhibitor, e.g. its ability to decrease the level and/or activity of the target can be determined, e.g. by measuring the level of target. Methods of measuring the numbers of a given type of cell and/or for measuring the level of an expression product or activity of a polypeptide are described elsewhere herein.

The term “agent” refers generally to any entity which is normally not present or not present at the levels being administered to a cell, tissue or subject. An agent can be selected from a group including but not limited to: polynucleotides; polypeptides; small molecules; and antibodies or antigen-binding fragments thereof. A polynucleotide can be RNA or DNA, and can be single or double stranded, and can be selected from a group including, for example, nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid aptamer, peptide-nucleic acid (PNA), locked nucleic acid (LNA), small organic or inorganic molecules; saccharide; oligosaccharides; polysaccharides; biological macromolecules, peptidomimetics; nucleic acid analogs and derivatives; extracts made from biological materials such as bacteria, plants, fungi, or mammalian cells or tissues and naturally occurring or synthetic compositions. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety selected, for example, from unsubstituted or substituted alkyl, aromatic, or heterocyclyl moieties including macrolides, leptomycins and related natural products or analogues thereof. Agents can be known to have a desired activity and/or property, or can be selected from a library of diverse compounds. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons. In some embodiments, the inhibitor can be an inhibitory nucleic acid; an antibody reagent; an antibody; or a small molecule.

A M2 macrophage inhibitor can be any agent that reduces the absolute and/or relative level (e.g. compared to the M1 phenotype) of M2 macrophages in a population of macrophages. As MaDAMs are a subset of M2 macrophages, in some embodiments, a M2 macrophage inhibitor can be a MaDAM inhibitor and vice versa.

In some embodiments, a M2 macrophage inhibitor can be a ROCK2 inhibitor. In some embodiments, a ROCK2 inhibitor can be a pan-ROCK inhibitor, e.g. it can inhibit ROCK1 and ROCK2. In some embodiments, a ROCK2 inhibitor can be a ROCK2-specific inhibitor, i.e. it can inhibit ROCK2 but not ROCK1. In addition to directly measuring ht expression and/or activity of ROCK1 and ROCK2, a ROCK2-specific inhibitor can be distinguished from a ROCK1 inhibitor or pan-ROCK inhibitor in that a ROCK2-specific inhibitor does not affect the cytoskeleton nor reduce recruitment.

Pan-ROCK inhibitors, e.g. agents that inhibit both ROCK1 and ROCK2 to a detectable degree are known in the art and include, without limitation Fasudil, HP1152P, CCG-1423; AS 1892802; GSk 269962; and Y-27632. Further non-limiting examples can include H-1152; N-(4-Pyridyl)-N′-(2,4,6-trichlorophenyl)urea; 3-(4-Pyridyl)-1H-indole (CAS 7272-84-6); (S)-(+)-2-Methyl-4-glycyl-1-(4-methylisoquinolinyl-5-sulfonyl)homopiperazine, 2HCl, H-1152, Glycyl, Glycyl H-1152 hydrochloride (CAS 913844-45-8); N-(4-(1H-pyrazol-4-yl)phenyl)-2,3-dihydrobenzo[b][1,4]dioxine-2-carboxamide (CAS 1072906-02-5); 1-(3-Hydroxybenzyl)-3-(4-(pyridin-4-yl)thiazol-2-yl)urea, Mesylate, 1-(3-Hydroxybenzyl)-3-(4-(pyridin-4-yl)thiazol-2-yl)urea, Methanesulfonate; RKI-1447; Glycyl-H 1152 dihydrochloride; HA 1100 hydrochloride; HA 1077, Dihydrochloride; hexahydro-1-(isoquinoline-5-sulfonyl)-1H-1,4-diazepine; AS1892802; Azabenzimidazole-aminofurazans including GSK269962A and SB772077; DE-104; SR 3677 dihydrochloride; GSK 429286; GSK269962; SB772077; SB-729743; SB-742548; BF 66851; BF 668522; BF 66853; 4-(1-aminoalkyl)-N-(4-pyridyl)cyclohexane-carboxamides; Y39983; Wf-536; HMN-1152; Rhostatin; BA-210; BA-207; BA-215; BA-285; BA-1037; Ki-23095; VAS-012; Thiazovivin; (+)-(R)-trans-4-(1-aminoethyl)-N-(4-piridyl)cyclohexanecarboxamide; 2-[4-(1H-indazol-5-yl)phenyl]-2-propanamine; N-(3-methoxybenzyl)-4-(4-piridyl)benzamide; 4-[(trans-4-aminocyclohexyl)amino]-2,5-difluorobenzamide; 4-[(trans-4-aminocyclohexyl)amino]-5-chloro-2-fluorobenzamide; 5-(hexahydro-1H-1,4-diazepin-1-ylsulfonyl)isoquinoline; N-(4-Pyridyl)-N′-(2,4,6-trichlorophenyl)urea; Y-27632; CCG-1423; AR-12286; K-115; Y-39983/SNJ-1656/RKI-983; INS-117548; DE-104; 4,5-c]pyridin-6-yl]oxy}phenyl)-4-{[2-(4-morpholinyl)ethyl]-oxy}benzamide; HA1077: 1-(5-isoquinolenesulfonyl)-homopiperazine; H1152P: (S)-(+)-2-methyl-14(4-methyl-5-isoquinolynyl)sulfonyl]homopiperazine; SB772077B: 4-(7-{[(3S)-3-amino-1-pyrrolidinyl]carbonyl}-1-ethyl-1Himidazo[4,5-c]pyridin-2-yl)-1,2,5-oxadiazol-3-amine; Wf536: (+)-(R)-4-(1-aminoethyl)-N-(4-pyridyl) benzamide; Y27632: (R)-(+)-trans-N-(4-pyridyl)-4-(1-aminoethyl)-cyclohexanecarboxamide; and Y39983: 44(1R)-aminoethyl]-N-(1H-pyrrolo [2,3-b]pyridine-4-yl)benzamide

Fasudil is obtainable from Asahi Kasei Pharma Corp (PMID: 3598899), Hydroxy fasudil is obtainable from Asahi Kasei Pharma Corp (PMID: 3598899), Y-39983 is obtainable from Novartis/Senju (PMID: 11606042) and Y27632 is obtainable from Mitsubishi Pharma (PMID: 9862451). (S)-(+)-2-Methyl-1-[(4-methyl-5-isoquinolinyl) sulfonyl]homopiperazine], N-(4-Pyridyl)-N′-(2,4,6-trichlorophenyl) urea and 3-(4-Pyridyl)-1H-indole are also available at AXXORA (UK) Ltd and other suppliers.

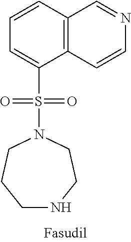 [SMILES: O=S(=O)(c1cccc2cnccc12)N1CCCNCC1]

ROCK inhibitors and methods of making them are described, e.g. in U.S. Pat. Nos. 8,071,779; 8,093,266; 7,199,147; US Patent Publications 2013/0131106; 2012/0178752; 2010/0041645; 2008/0161297; 2012/0270868; 2009/0203678; 2010/0137324; 2013/0131059; International Patent Publications WO 2009/155209; WO 2012/135697; WO 2005/003101; European Patents 2628482, 1256578; 1270570; and 1550660; and Tamura et al. Biophys Ada 2005 1754:245-252; each of which is incorporated by reference herein in its entirety. Further exemplary ROCK inhibitors are described in, e.g., WO 98/06433; isoquinoline sulfonyl derivatives disclosed in WO 97/23222, Nature 389, 990-994 (1997) and WO 99/64011; heterocyclic amino derivatives disclosed in WO owl/56988; indazole derivatives disclosed in WO 02/100833; and quinazoline derivatives disclosed in WO 02/076976 and WO 02/076977; in WO02053143, p. 7, lines 1-5, EP1163910 A1, p. 3-6, WO02076976 A2, p. 4-9, preferably the compounds described on p. 10-13 and p. 14 lines 1-3, WO02/076977A2, the compounds I-VI of p. 4-5, WO03/082808, p. 3-p. 10 (until line 14), the indazole derivates described in U.S. Pat. No. 7,563,906 B2, WO2005074643A2, p. 4-5 and the specific compounds of p. 10-11, WO2008015001, pages 4-6, EP1256574, claims 1-3, EP1270570, claims 1-4, and EP 1 550 660; each of which is incorporated by reference herein in its entirety. ROCK inhibitors can also comprise inhibitory nucleic acids, e.g. siRNAs, miRNAS, amiRNAs, shRNAs and the like. In some embodiments, the following validated ROCK I/II-specific siRNA molecules may be used: ON-TARGET PLUS siRNA human ROCK I (ID: L-003536-00; Dharmacon); ON-TARGET PLUS siRNA human ROCK II (ID: L-004610-00; Dharmacon).

ROCK2-specific inhibitors are known in the art and include, without limitation SLx2119 and related compound and XD-4000 (see, e.g. Liao et al. 2007 J Cardiovasc Pharamcol 50:17-24; which is incorporated by reference herein in its entirety). In some embodiments, the ROCK2-specific inhibitor can be SLx2119. SLx2119 and related compounds are described in International Patent Publication WO2010/104851 and US Patent Publication 2012/0202793; each of which is incorporated by reference herein in its entirety.

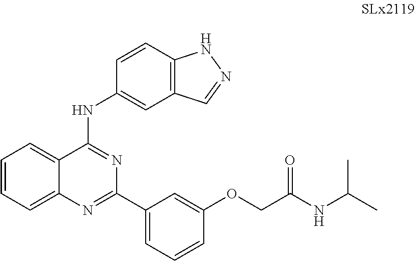 [SMILES: CC(C)NC(=O)COc1cccc(-c2nc(Nc3ccc4[nH]ncc4c3)c3ccccc3n2)c1]

ROCK1-specific inhibitors are known in the art and include, without limitation, GSK 429286, dihydropyrimidinones and derivatives thereof, and dihydropyrimidines and derivatives thereof, (including compounds as described in Sehon et al. J. Med. Chem., 2008, 51 (21), pp 6631-6634) and the inhibitors disclosed in US Patent Publication 2006/0142193; each of which is incorporated by reference herein in its entirety.

In some embodiments, the M2 macrophage inhibitor can be a M1-promoting cytokine. M1-promoting cytokines are known in the art and can include, but are not limited to INF-γ. LPS, TNF-α, IL-23, IL-12, and IL-1β. In some embodiments, more than one M1-promoting cytokine can be used, e.g. both INF-γ and LPS can be administered.

In one aspect, described herein is a method of treating pathogenic angiogenesis, vascular leakage, aging, or age-related conditions, the method comprising administering a M2 or MaDAM macrophage inhibitor to a subject. In some embodiments, the pathogenic angiogenesis or vascular leakage can be associated with AMD or choroidal neovascularization (CNV). In some embodiments, the pathogenic angiogenesis or vascular leakage can be associated with aging. In one aspect, described herein is a method of treating M2 macrophage-associated disorders, the method comprising administering a M2 or MaDAM macrophage inhibitor to a subject. As demonstrated herein, M2 macrophages increase as a tissue ages, and thus the methods described herein can counteract the effects of aging and/or prevent or treat conditions linked to aging, e.g. AMD. Other M2 macrophage-associated conditions are known in the art and can include, by way of non-limiting example, Ommen's syndrome, AMD, pathogeneic angiogenesis; chronic GVDH, atopic disorders, asthma, eczema, allergic rhinitis, some systemic autoimmune diseases, progressive systemic sclerosis, systemic lupus erythematosus, and allergies.

In some embodiments, the M2 macrophage inhibitor can be a pan-ROCK inhibitor. In some embodiments, the M2 macrophage inhibitor can be a M1-promoting cytokine.

As described elsewhere herein, the administration of M1 macrophages can reduce the level of M2 macrophages and induce therapeutic changes similar to the administration of an M2 macrophage inhibitor. In one aspect, described herein is a method of treating pathogenic angiogenesis or vascular leakage, the method comprising administering a M1 macrophage to a subject. In some embodiments, the pathogenic angiogenesis is associated with a condition selected from the group consisting of AMD, CNV, or aging. In some embodiments, the pathogenic vascular leakage is associated with AMD. In some embodiments, the M1 macrophage is administered via intravitreal injection. In some embodiments, the M1 macrophage can from the same species as the subject receiving the treatment. In some embodiments, the M1 macrophage can be autologous to the subject receiving the treatment. In some embodiments, the M1 macrophage syngenic to the subject receiving the treatment.

In one aspect, described herein is a method of promoting macrophage differentiation to the M2 phenotype, the method comprising contacting a macrophage with a M1 macrophage inhibitor. In some embodiments, the M1 macrophage inhibitor can be a ROCK1 inhibitor. In some embodiments, the ROCK1 inhibitor can be a ROCK1-specific inhibitor, i.e. it can inhibit ROCK1 but not ROCK2. In addition to directly measuring the expression and/or activity of ROCK1 and ROCK2, a ROCK1 inhibitor can be distinguished from a ROCK2-specific inhibitor in that a ROCK1 inhibitor does affect the cytoskeleton and reduces recruitment.

In some embodiments, the M1 macrophage inhibitor can be a M2-promoting cytokine. M2-promoting cytokines are known in the art and can include, but are not limited to TGF-β, IL-4, IL-10, and IL-13. In some embodiments, more than one M2-promoting cytokine can be used, e.g. all of IL-4, IL-10, and IL-13 can be administered.

An overabundance of M1 macrophages is known to contribute to Th1-associated diseases, autoimmune diseases, and/or inflammatory disease. M1 macrophage-associated diseases are known in the art and can include but are not limited to, e.g., Ankylosing spondylitis; atherosclerosis; Barrett's esophagus; Chronic Lyme disease (borreliosis); Crohn's disease; Diabetes insipidus; Diabetes type I; Diabetes type II; Fibromyalgia (FM); Gastroesophageal reflux disease (GERD); Hypertension; Irritable Bowel Syndrome (IBS); Interstitial cystitis (IC); Kidney stones; Lofgren's syndrome; Lupus erythematosis; Multiple Chemical Sensitivity (MCS); Multiple sclerosis; Myasthenia gravis; Neuropathy; Osteoarthritis; Polymyalgia rheumatica; Psoriasis; Psoriatic arthritis; Reactive arthritis (Reiter syndrome); Rheumatoid arthritis; Scleroderma; Ulcerative colitis; Uveitis; chronic ulcers; chronic venous ulcers; infectious mononucleosis, and organ-specific autoimmune diseases.

Accordingly, in one aspect, described herein is a method of treating an inflammatory or autoimmune disease, the method comprising administering a M1 macrophage inhibitor to a subject. In some embodiments, the M1 macrophage inhibitor is a ROCK1 inhibitor. In some embodiments, the ROCK1 inhibitor is a ROCK1-specific inhibitor.

In some embodiments of any of the aspects described herein, the macrophage or ROCK1 inhibitor is not a direct modulator of VEGF-A, e.g. is it not an agent that binds to VEGF-A or an expression product thereof. Non-limiting examples of direct modulators of VEGF include e.g. bevacizumab (AVASTIN®), ranibizumab (LUCENTIS®), afillbercept (ELYEA®), and pegaptanib (MACUGEN®).

In some embodiments, the methods described herein relate to treating a subject having or diagnosed as having, e.g. macular degeneration, e.g. AMD. Subjects having AMD can be identified by a physician using current methods of diagnosing AMD. Symptoms and/or complications of AMD which characterize these conditions and aid in diagnosis are well known in the art and include but are not limited to, drusen, pigmentary alterations, exudative changes in the eye, atrophy, drastic decreases in visual acuity, blurred vision, central sarcoma, and distorted vision. Tests that may aid in a diagnosis of, e.g. AMD include, but are not limited to, fluorescein angiography and optical coherence tomography. A family history of AMD, or exposure to risk factors for AMD (e.g. age, genetics, hypertension, high cholesterol, etc.) can also aid in determining if a subject is likely to have AMD or in making a diagnosis of AMD.

The compositions and methods described herein can be administered to a subject having or diagnosed as having a condition described herein, e.g. AMD. In some embodiments, the methods described herein comprise administering an effective amount of compositions described herein, to a subject in order to alleviate a symptom of a disease or condition. As used herein, “alleviating a symptom of a disease or condition” is ameliorating any condition or symptom associated with the disease or condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a composition, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for macrophage differentiation, among others. The dosage can be determined by a physician and adjusted, as necessary, to suit observed effects of the treatment.

In some embodiments, the technology described herein relates to a pharmaceutical composition comprising a composition as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include an anti-VEGF-A treatment, (e.g. bevacizumab (AVASTIN®), ranibizumab (LUCENTIS®), afillbercept (ELYEA®), and/or pegaptanib (MACUGEN®)) and/or a laser therapy.

In certain embodiments, an effective dose of a composition comprising an agent as described herein can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the composition. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the active ingredient, its potency, and the extent to which symptoms, markers, or indicators of a condition described herein are desired to be reduced, for example the percentage reduction desired for one or more symptoms (e.g. vision loss or pathogenic angiogenesis). The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein (e.g. a decrease in pathogeneic angiogenesis) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. angiogenesis. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. the level of angiogenesis or a marker of ROCK1 or ROCK2 signaling). It is well within the ability of one skilled in the art to monitor efficacy of administration and/or treatment by measuring any one of such parameters, or any combination of parameters. Efficacy can be assessed in animal models of a condition described herein, for example treatment of AMD. When using an experimental animal model, efficacy of treatment is evidenced when a statistically significant change in a marker is observed, e.g. ROCK2 signaling or angiogenesis levels.

In vitro and animal model assays are provided herein which allow the assessment of a given dose of a composition described herein, e.g, a ROCK2 inhibitor. By way of non-limiting example, the effects of a dose can be assessed by contacting cells in vitro with a ROCK2 inhibitor and detecting the level of ROCK1/ROCK2 marker polypeptides. A non-limiting example of a protocol for such an assay is as follows: cells contacted with the inhibitor are treated with lysis buffer (mammalian cell lysis kit MCL1, Sigma), supplemented with protease and phosphatase inhibitors (P2850, P5726, P8340 Sigma), and sonicated. The lysate can be centrifuged (12000 rpm, 15 min, 4° C.) and the supernatant collected. Each sample containing an equal amount of total protein, quantified by protein assay (Bio-Rad Laboratories), can be separated by SDS-PAGE and electroblotted to PVDF membranes (Invitrogen). To block nonspecific binding, the membranes can be washed with 5% skim milk and subsequently incubated with one or more of the following: rabbit Abs against phospho-MBS/MYPT1-THr853 (CY-P1025, Cyclex), MYPT1 (sc-25618, Santa Cruz Biotechnology), phospho NF-κB p65 (3033, Cell Signaling), NF-κB p65 (3034, Cell Signaling), IκB-α (9242, Cell Signaling) or mouse Abs against pIκB-α (9246, Cell Signaling), ROCK1 and ROCK2 (611136, 610623, BD Transduction Laboratories), pERM (3149, Cell Signaling), ERM (3142, Cell Signaling), IL-4 (ab11524, Abcam), CD163 (sc-33560, Santa Cruz Biotechnology), CCR3 (ab32512, Abcam), CCR7 (ab65851, Abcam), CD80 (ab53003, Abcam) and β-tubulin (ab11308, Abcam) at 4° C. overnight, followed by incubation with a horseradish peroxidase-conjugated donkey or sheep Ab against rabbit or mouse IgG (NA934V, NXA931, GE Healthcare), or goat antirat secondary (goat anti-rat IgG-HRP: sc-2032, Santa Cruz). The signals can be visualized by chemiluminescence (ECL kit; GE Healthcare) according to the manufacturer's protocol.

The efficacy of a given dosage combination can also be assessed in an animal model, e.g. a rat model of CNV. For example, Brown Norway rats can be subjected to laser injury to induce CNV. 7 and/or 14 days after laser injury, the rats are anesthetized, and fluorescein angiography performed using a digital fundus camera (SLO; HRA2; Heidelberg Engineering). Fluorescein injections can be performed intravenously (0.2 ml of 2% fluorescein; Akorn, NDC 17478-253-10). FA images can be evaluated by two masked retina specialists. The grading criteria can be as follows: Grade-0, no hyperfluorescence; Grade-I, hyperfluorescence without leakage; Grade-IIA, hyperfluorescence in the early or mid-transit images and late leakage; Grade-IIB, bright hyperfluorescence in the transit images and late leakage beyond the treated areas.

The role of M2 (and particularly MaDAM) macrophages in pathogenic angiogenesis and vascular leakage, as described herein, permits macrophage differentation to be used as a biomarker of these processes. Accordingly, described herein is a method of determining if a tissue is affected by pathogenic angiogenesis or vascular leakage, the method comprising measuring the level of M2 or MaDAM cells present in the tissue; and determining the tissue is affected by pathogenic angiogenesis if the level of M2 or MaDAM cells is increased relative to a control. In some embodiments, the pathogenic angiogenesis or vascular leakage is associated with AMD or CNV.

Markers for M1, M2, and/or MaDAM cells are provided elsewhere herein. The level of a given type of macrophage can be determined by contacting a sample with a reagent specific for a marker of a particular cell type and detecting the level of the reagent which is bound or hybridized to a target. Alternatively, the number of cells bound and/or hybridized by the reagent can be measured, e.g., by FACS or microscopy. In some embodiments, the level of M2 or MaDAM cells is determined by measuring the level of CD11b. In some embodiments, the level of M2 or MaDAM cells is determined by measuring the level of CD163. In some embodiments, the level of M2 or MaDAM cells is determined by measuring the level of CD206. In some embodiments, the level of the marker can be the number of cells comprising a detectable level of the marker.

In some embodiments, the level of M2 or MaDAM cells is determined by measuring the level of ROCK1 or ROCK2. In some embodiments, an increased level of ROCK2 expression products (e.g. ROCK2 polypeptide or an mRNA encoding a ROCK2 polypeptide) and/or a decreased level of ROCK1 expression products is indicative of an increased level of M2 and/or MaDAM cells. In some embodiments, the distribution of ROCK2 polypeptide in the cytoplasm of macrophages indicates an increased level of M1 macrophages, while a concentration of ROCK2 polypeptide near the nucleus of macrophages indicates an increased level of M2 and/or MaDAM cells.

Additional markers of M2 and/or MaDAM cells are provided herein, e.g. molecules involved in ROCK2 signaling. In some embodiments, the level of M2 and/or MaDAM cells is determined by measuring the level of a marker selected from the group consisting of: arginase 1; YM 1; Fizz1; CCL5; IL-10; CCL3; MYPT1; IκBa; NF-κB; IL-4; CCR3; MLC; RhoA; and iNOS. In some embodiments, an increased level of a marker selected from the group consisting of arginase 1; YM 1; Fizz1; IL-10; MYPT1; IκBa; NF-κB; IL-4; CCR3; MLC; and RhoA; indicates an increases level of ROCK2 signaling or an increased level of M2 and/or MaDAM cells. In some embodiments, an increased level of phosphorylation of a marker selected from the group consisting of MYPT1; IκBa; NF-κB; IL-4; and MLC indicates an increases level of ROCK2 signaling or an increased level of M2 and/or MaDAM cells. In some embodiments, an increased level of a marker selected from the group consisting of CCL5; CCL3; and iNOS indicates a decreased level of ROCK2 signaling or a decreased level of M2 and/or MaDAM cells. Additional markers for M2 macrophages and/or MaDAMs are known in the art, see, e.g., Mantovani et al. Trends Immunol 2004 25:677-686; which is incorporated by reference herein in its entirety. In some embodiments, a decreased level of ROCK1 activity (e.g. a decreased level of M1 macrophages) is indicated by increased levels of IL-4 and/or CCL22.

Methods for measuring the level of a particular mRNA and/or polypeptide are known to one of skill in the art, e.g. RTPCR with primers specific for the target mRNA can be used to determine the level of the mRNA and Western blotting with an anti-target antibody (e.g. anti-CD206 Cat No. ab8918; Abcam; Cambridge, Mass.) can be used to determine the level of the target polypeptide. The activity of, e.g., ROCK1 and ROCK2 can be determined using methods known in the art, including, by way of non-limiting example, by measuring the level of the markers described herein, and/or the level of phosphorylation of the markers described herein (e.g., with a phospho-specific antibody reagent).

In some embodiments, the methods and assays described herein include (a) transforming the target molecule, e.g., a target gene expression product into a detectable gene target; (b) measuring the amount of the detectable gene target; and (c) comparing the amount of the detectable gene target to an amount of a reference, wherein if the amount of the detectable gene target is statistically significantly different than the amount of the reference level, a level of M1, M2, and/or MaDAMs is determined and/or a level of ROCK1 and/or ROCK2 expression and/or activity is determined.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

Methods to measure gene expression products associated with the marker genes described herein are well known to a skilled artisan. Such methods to measure gene expression products, e.g., protein level, include ELISA (enzyme linked immunosorbent assay), western blot, FACS, radioimmunological assay; (RIA); sandwich assay; fluorescent in situ hybridization (FISH); immunohistological staining; immunoelectrophoresis; immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques.

For example, antibodies for the polypeptide expression products of the marker genes described herein are commercially available and can be used for the purposes of the invention to measure protein expression levels. Alternatively, since the amino acid sequences for the marker genes described herein are known and publically available at NCBI website, one of skill in the art can raise their own antibodies against these proteins of interest for the purpose of the invention. The amino acid sequences of the marker genes described herein have been assigned NCBI accession numbers for different species such as human, mouse and rat.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (i.e. a marker gene polypeptide as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., tumor, blood, serum or urine) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tumor samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, an adaptation of Western blotting (Towbin et al., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

Flow cytometry is a well-known technique for analyzing and sorting cells (or other small particles) suspended in a fluid stream. This technique allows simultaneous analysis of the physical and/or chemical characteristics of single cells flowing through an optical, electronic, or magnetic detection apparatus. As applied to FACS, the flow cytometer consists of a flow cell which carries the cells in a fluid stream in single file through a light source with excites the fluorescently labeled detection marker(s) (for example, antibody reagents) and measures the fluorescent character of the cell. The fluid stream is then ejected through a nozzle and a charging ring, under pressure, which breaks the fluid into droplets. The flow cell device and fluid stream is calibrated such that there is a relatively large distance between individual cells or bound groups of cells, resulting in a low probability that any droplet contains more than a single cell or bound group of cells. The charging ring charges the droplets based on the fluorescence characteristic of the cell which is contained therein. The charged droplets are then deflected by an electrostatically-charged deflection system which diverts the droplets into various containers based upon their charge (related to the fluorescence intensity of the cell). A FACS system (e.g. the FACSARIA™ flow cytometer (BD Biosciences) and FLOWJO™ Version 7.6.4 (TreeStar)) can detect and record the number of total cells as well as the number of cells which display one or more fluorescent characteristics, e.g. the total number of cells bound by one or more antibody reagents specific for a CTC marker gene.

In certain embodiments, the gene expression products as described herein can be instead determined by determining the level of messenger RNA (mRNA) expression of genes associated with the marker genes described herein. Such molecules can be isolated, derived, or amplified from a biological sample, such as a tumor biopsy. Detection of mRNA expression is known by persons skilled in the art, and comprise, for example but not limited to, PCR procedures, RT-PCR, quantitative PCR or RT-PCR, Northern blot analysis, differential gene expression, RNA protection assay, microarray analysis, hybridization methods, next-generation sequencing etc. Non-limiting examples of next-generation sequencing technologies can include Ion Torrent, Illumina, SOLiD, 454; Massively Parallel Signature Sequencing solid-phase, reversible dye-terminator sequencing; and DNA nanoball sequencing.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art. The nucleic acid sequences of the marker genes described herein have been assigned NCBI accession numbers for different species such as human, mouse and rat. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the nucleic acid molecule to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylene diaminetetraacetic acid (EDTA).

In some embodiments of any of the aspects described herein, the level of expression products of more than one gene can be determined simultaneously (e.g. a multiplex assay) or in parallel. In some embodiments, the level of expression products of no more than 200 other genes is determined. In some embodiments, the level of expression products of no more than 100 other genes is determined. In some embodiments, the level of expression products of no more than 20 other genes is determined. In some embodiments, the level of expression products of no more than 10 other genes is determined.

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments, a test sample can comprise cells from subject.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject.

In some embodiments, the assay or method described herein can further comprise creating a report based on the level or number of M1 and/or M2 macrophages and/or the expression level of ROCK1, ROCK2, or a marker thereof.

In some embodiments, the method further comprises a step of administering a treatment for pathogenic angiogenesis if the level of M2 or MaDAM cells is increased relative to the control. In some embodiments, the treatment is a treatment as described herein, e.g. an M2 macrophage inhibitor and/or M1 macrophage cells. In some embodiments, the treatment can be an anti-VEGF treatment.

TABLE 1

| | NCBI Gene | mRNA NCBI | | Polypeptide | |
|---|---|---|---|---|---|
| Gene Name | ID No: | Seq Ref: | SEQ ID NO: | NCBI Seq Ref: | SEQ ID NO: |
| ROCK1 | 6093 | NM_005406 | 1 | NP_005397 | 2 |
| ROCK2 | 9475 | NM_004850 | 3 | NP_004841 | 4 |
| INF-γ | 3458 | NM_000619 | 5 | NP_000610 | 6 |
| CD11b | 3684 | NM_001145808 | 7 | NP_001139280 | 8 |
| CD163 | 9332 | NM_004244 | 9 | NP_004235 | 10 |
| CD206 | 4360 | NM_002438 | 11 | NP_002429 | 12 |
| IL-4 | 3565 | NM_000589 | 13 | NP_000580 | 14 |
| IL-10 | 3586 | NM_000572 | 15 | NP_000563 | 16 |
| IL-13 | 3596 | NM_002188 | 17 | NP_002179 | 18 |
| TNF-α | 7124 | NM_000594 | 19 | NP_000585 | 20 |
| IL-23 | 51561 | NM_016584 | 21 | NP_057668 | 22 |
| IL-12 | 3593 | NM_002187 | 23 | NP_002178 | 24 |
| IL-1β | 3553 | NM_000576 | 25 | NP_000567 | 26 |
| CCL3 | 6348 | NM_002983 | 27 | NP_002974 | 28 |
| CCL5 | 6352 | NM_001278736 | 29 | NP_001265665 | 30 |
| CD80 | 941 | NM_005191 | 31 | NP_005182 | 32 |
| CCR7 | 1236 | NM_001838 | 33 | NP_001829 | 34 |
| iNOS | 4843 | NM_000625 | 35 | NP_000616 | 36 |
| TGF-β | 7040 | NM_000660 | 37 | NP_000651 | 38 |
| CCL22 | 6367 | NM_002990 | 39 | NP_002981 | 40 |
| YM 1 | 12655 | NM_009892 | 41 | NP_034022 | 42 |
| Fizz1 | 84666 | NM_032579 | 43 | NP_115968 | 44 |
| Arginase 1 | 383 | NM_001244438 | 45 | NP_001231367 | 46 |
| MYPT1 | 4659 | NM_001143885 | 47 | NP_001137357 | 48 |
| IκBa | 4792 | NM_020529 | 49 | NP_065390 | 50 |
| NF-κB | 4790 | NM_003998 | 51 | NP_003989 | 52 |
| CCR3 | 1232 | NM_001164680 | 53 | NP_001158152 | 54 |
| MLC | 23209 | NM_015166 | 55 | NP_055981 | 56 |
| RhoA | 387 | NM_001664 | 57 | NP_001655 | 58 |

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g., AMD. A subject can be male or female. In some embodiments, the subject can be a human subject.

In some embodiments, the subject can be a subject in need of treatment for a condition described herein, e.g. pathogenic angiogenesis, AMD, and/or CNV.

A subject can be one who has been previously diagnosed with or identified as suffering from or having a condition in need of treatment (e.g. AMD) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, “expression level” refers to the number of mRNA molecules and/or polypeptide molecules encoded by a given gene that are present in a cell or sample. Expression levels can be increased or decreased relative to a reference level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein an “antibody” refers to IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target molecule.

Aptamers are short synthetic single-stranded oligonucleotides that specifically bind to various molecular targets such as small molecules, proteins, nucleic acids, and even cells and tissues. These small nucleic acid molecules can form secondary and tertiary structures capable of specifically binding proteins or other cellular targets, and are essentially a chemical equivalent of antibodies. Aptamers are highly specific, relatively small in size, and non-immunogenic. Aptamers are generally selected from a biopanning method known as SELEX (Systematic Evolution of Ligands by Exponential enrichment) (Ellington et al. Nature. 1990; 346(6287):818-822; Tuerk et al., Science. 1990; 249(4968):505-510; Ni et al., Curr Med Chem. 2011; 18(27):4206-14; which are incorporated by reference herein in their entireties). Methods of generating an apatmer for any given target are well known in the art. Preclinical studies using, e.g. aptamer-siRNA chimeras and aptamer targeted nanoparticle therapeutics have been very successful in mouse models of cancer and HIV (Ni et al., Curr Med Chem. 2011; 18(27):4206-14).

Inhibitors of the expression of a given gene can be an inhibitory nucleic acid. In some embodiments, the inhibitory nucleic acid is an inhibitory RNA (iRNA). Non-limiting examples of inhibitory RNAs can include siRNAs, dsRNAs, amiRNAs, miRNAs, shRNA and variants, fragments, and/or modifications thereof. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of the target. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 564,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O-CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. AMD. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating a condition selected from the group consisting of:
   pathogenic angiogenesis; vascular leakage; and aging or age-related conditions;
   the method comprising administering a M2 or MaDAM macrophage inhibitor to a subject.
2. The method of paragraph 1, wherein the M2 or MaDAM macrophage is a CD11b(+) cell.
3. The method of any of paragraphs 1-2, wherein the M2 or MaDAM macrophage is a CD163(+) cell.
4. The method of any of paragraphs 1-3, wherein the M2 or MaDAM macrophage is a CD206(+) cell.
5. The method of any of paragraphs 1-4, wherein the pathogenic angiogenesis is associated with a condition selected from the group consisting of:
   AMD, CNV, or aging.
6. The method of any of paragraphs 1-5, wherein the vascular leakage is associated with AMD.
7. The method of any of paragraphs 1-6, wherein the M2 macrophage inhibitor is a pan-ROCK inhibitor.
8. The method of paragraph 7, wherein the pan-ROCK inhibitor is selected from the group consisting of:
   Fasudil; HP1152P; and Y-27632.
9. The method of any of paragraphs 1-8, where in the M2 macrophage inhibitor is a ROCK2-specific inhibitor.
10. The method of any of paragraphs 1-9, wherein the ROCK2 inhibitor does not inhibit ROCK1.
11. The method of any of paragraphs 1-10, wherein the ROCK2 inhibitor does not affect the cytoskeleton.
12. The method of any of paragraphs 1-11, wherein the ROCK2 inhibitor does not reduce recruitment.
13. The method of any of paragraphs 1-12, wherein the ROCK2 inhibitor is SLx2119.
14. The method of any of paragraphs 1-13, wherein the M2 macrophage inhibitor is a M1-promoting cytokine.
15. The method of paragraph 14, wherein the M1-promoting cytokine is selected from the group consisting of:
   INF-γ and LPS.
16. The method of any of paragraphs 1-15, wherein the inhibitor is not a direct modulator of VEGF-A.
17. A method of treating a condition selected from the group consisting of:
   pathogenic angiogenesis; vascular leakage; and aging or age-related conditions;
   the method comprising administering a M1 macrophage to a subject.
18. The method of paragraph 17, wherein the pathogenic angiogenesis is associated with a condition selected from the group consisting of:
   AMD, CNV, or aging.
19. The method of any of paragraphs 17-18, wherein the pathogenic vascular leakage is associated with AMD.
20. The method of any of paragraphs 17-19, wherein the M1 macrophage is administered via intravitreal injection.
21. A method of promoting macrophage differentiation to the M1 phenotype, the method comprising contacting a macrophage with M2 macrophage inhibitor.
22. The method of paragraph 21, where in the inhibitor is a ROCK2-specific inhibitor.
23. The method of any of paragraphs 21-22, wherein the ROCK2 inhibitor does not inhibit ROCK1.
24. The method of any of paragraphs 21-23, wherein the ROCK2 inhibitor does not affect the cytoskeleton.
25. The method of any of paragraphs 21-24, wherein the ROCK2 inhibitor does not reduce recruitment.
26. The method of any of paragraphs 21-25, wherein the ROCK2 inhibitor is SLx2119.
27. The method of any of paragraphs 21-26, wherein the M2 macrophage inhibitor is a M1-promoting cytokine.
28. The method of paragraph 27, wherein the M1-promoting cytokine is selected from the group consisting of:
   INF-γ and LPS.
29. The method of any of paragraphs 21-28, wherein the inhibitor is not a direct modulator of VEGF-A.
30. A method of treating an inflammatory or autoimmune disease the method comprising administering a M1 macrophage inhibitor to a subject.
31. The method of paragraph 30, wherein the M1 macrophage inhibitor is a ROCK1 inhibitor.
32. The method of paragraph 31, where in the ROCK1 inhibitor is a ROCK1-specific inhibitor.
33. The method of any of paragraphs 30-32, wherein the ROCK1 inhibitor does not inhibit ROCK2.
34. The method of any of paragraphs 30-33, wherein the ROCK1 inhibitor does affect the cytoskeleton.
35. The method of any of paragraphs 30-34, wherein the ROCK1 inhibitor reduces recruitment.
36. The method of any of paragraphs 30-35, wherein the ROCK1 inhibitor is selected from the group consisting of:
   GSK 429286; a dihydropyrimidinone; and a dihydropyrimidine.
37. The method of any of paragraphs 30-36, wherein the M1 macrophage inhibitor is a M2-promoting cytokine.
38. The method of paragraph 37, wherein the M2-promoting cytokine is selected from the group consisting of:
   IL-4; IL-10; and IL-13.
39. The method of any of paragraphs 30-38, wherein the inhibitor is not a direct modulator of VEGF-A.
40. A method of promoting macrophage differentiation to the M2 phenotype, the method comprising contacting a macrophage with a M1 macrophage inhibitor.
41. The method of paragraph 40, wherein the M1 macrophage inhibitor is a ROCK1 inhibitor.
42. The method of paragraph 41, where in the ROCK1 inhibitor is a ROCK1-specific inhibitor.
43. The method of any of paragraphs 40-42, wherein the ROCK1 inhibitor does not inhibit ROCK2.
44. The method of any of paragraphs 40-43, wherein the ROCK1 inhibitor does affect the cytoskeleton.
45. The method of any of paragraphs 40-44, wherein the ROCK1 inhibitor reduces recruitment.
46. The method of any of paragraphs 40-45, wherein the ROCK1 inhibitor is selected from the group consisting of:
   GSK 429286; a dihydropyrimidinone; and a dihydropyrimidine.
47. The method of any of paragraphs 40-46, wherein the M1 macrophage inhibitor is a M2-promoting cytokine.
48. The method of paragraph 47, wherein the M2-promoting cytokine is selected from the group consisting of:
   IL-4; IL-10; and IL-13.
49. The method of any of paragraphs 40-48, wherein the inhibitor is not a direct modulator of VEGF-A.
50. A method of determining if a tissue is affected by pathogenic angiogenesis or vascular leakage, the method comprising:
   measuring the level of M2 or MaDAM cells present in the tissue; and
   determining the tissue is affected by pathogenic angiogenesis if the level of M2 or MaDAM cells is increased relative to a control.
51. The method of paragraph 50, wherein the pathogenic angiogenesis or vascular leakage is associated with AMD or CNV.
52. The method of any of paragraphs 50-51, wherein the level of M2 or MaDAM cells is determined by measuring the level of CD11b.
53. The method of any of paragraphs 50-52, wherein the level of M2 or MaDAM cells is determined by measuring the level of CD163.
54. The method of any of paragraphs 50-53, wherein the level of M2 or MaDAM cells is determined by measuring the level of CD206.
55. The method of any of paragraphs 50-54, wherein the level of M2 or MaDAM cells is determined by measuring the level of ROCK1 or ROCK2.
56. The method of any of paragraphs 50-55, wherein the level of M2 or MaDAM cells is determined by measuring the level of a marker selected from the group consisting of:
   arginase 1; YM 1; Fizz1; CCL5; IL-10; CCL3; MYPT1; IκBa; NF-κB; IL-4; CCR3; MLC; RhoA; and iNOS.
57. The method of any of paragraphs 50-56, wherein the method further comprises a step of administering a treatment for pathogenic angiogenesis if the level of M2 or MaDAM cells is increased relative to the control.
58. The method of paragraph 57, wherein the treatment is the treatment of any of paragraphs 1-20.
59. A method of treatment comprising administering a M2 or MaDAM macrophage inhibitor to a patient determined to have an increased level of M2 or MaDAM cells in a tissue.
60. The method of paragraph 59, wherein the M2 or MaDAM macrophage is a CD11b(+) cell.
61. The method of any of paragraphs 59-60, wherein the M2 or MaDAM macrophage is a CD163(+) cell.
62. The method of any of paragraphs 59-61, wherein the M2 or MaDAM macrophage is a CD206(+) cell.
63. The method of any of paragraphs 59-62, wherein the patient is a patient in need of treatment for a condition selected from the group consisting of:
   vascular leakage; pathogenic angiogenesis; AMD, CNV, and aging.
64. The method of any of paragraphs 59-63, wherein the M2 macrophage inhibitor is a pan-ROCK inhibitor.
65. The method of paragraph 64, wherein the pan-ROCK inhibitor is selected from the group consisting of:
   Fasudil; HP1152P; and Y-27632.
66. The method of any of paragraphs 59-65, where in the M2 macrophage inhibitor is a ROCK2-specific inhibitor.
67. The method of any of paragraphs 59-66, wherein the ROCK2 inhibitor does not inhibit ROCK1.
68. The method of any of paragraphs 59-67, wherein the ROCK2 inhibitor does not affect the cytoskeleton.
69. The method of any of paragraphs 59-68, wherein the ROCK2 inhibitor does not reduce recruitment.
70. The method of any of paragraphs 59-69, wherein the ROCK2 inhibitor is SLx2119.
71. The method of any of paragraphs 59-70, wherein the M2 macrophage inhibitor is a M1-promoting cytokine.
72. The method of paragraph 71, wherein the M1-promoting cytokine is selected from the group consisting of:
   INF-γ and LPS.
73. The method of any of paragraphs 59-72, wherein the inhibitor is not a direct modulator of VEGF-A.
74. The method of any of paragraphs 59-63, wherein the M2 or MaDAM macrophage inhibitor is a M1 macrophage.
75. The method of paragraph 74, wherein the M1 macrophage is administered via intravitreal injection.

### EXAMPLES

### Example 1

A mechanistic link between age and macular degeneration has not been established. Described herein is a novel regulatory role for the rho-associated kinase (ROCK) in macrophage polarization and aging. ROCK1 inhibition increased the M2-specific molecules, CCL22 and IL-4. In contrast, ROCK2 inhibition increased the M1-specific molecules, CCL-3, CCL-5, INFγ, as well as CD80+ cells, while reducing M2-specific IL-10 and CD206+ cells. A distinct M2-like population, the Macular Degeneration Associated Macrophages (MaDAMs) that expressed both ROCK isoforms was found in human, primate, and rodent choroidal neovascular (CNV) lesions, a hallmark of age-related macular degeneration (AMD). ROCK2 inhibition with the novel ROCK2 specific inhibitor, SLx2119, significantly decreased MaDAMs, CNV size, and leakage. In contrast, CNV was unchanged in ROCK1+/−TieCre mice. M2, but not M1, macrophages exacerbated CNV, indicating the causal role of these cells in AMD pathology.

In WT mice, age increased pMYPT-1, and particularly pIκB-a, downstream of ROCK, as well as M2 characteristics, such as IL-4 and CCR3. IκB-α activity turned out a biomarker for aging, while CD163 in WT only concurred with CNV. In contrast, the M1 markers CCR7 and CD80 remained unaffected by age or CNV. In aged MCP-1−/− mice, elevated IL-4, pIκB-a, ERM, and pERM underline the importance of ROCK signaling and M2 shift in AMD. This work reveals a novel connection between age, ROCK signaling as cause of M2-like macrophage polarization, and AMD pathology. The ROCK signaling pathway provides attractive targets for immunity and anti-aging.

### Example 2

ROCK1/2 signaling is described herein as a master switch in macrophage polarization and identify the M2-like [age-related] macular-degeneration associated macrophages (MaDAMs) as a cause of disease. As a source of VEGF¹⁴, macrophages promote angiogenesis and CNV⁸⁻¹³. Paradoxically however, macrophages also inhibit CNV⁸. The work presented herein resolves this apparent discrepancy and provides novel mechanistic insights into how macrophages both decrease and increase CNV, depending on their polarization.

As opposed to M1 macrophages that reduce CNV, M2 macrophages increase CNV. ROCK2 inhibition suppresses M2-like differentiation, while it furthers M1 polarization, the latter even in an M2 environment. In contrast, ROCK1 inhibition furthers M2 polarization, revealing a decisive role for ROCK-isoforms in macrophage differentiation.

In mouse, monkey, and man, ROCK expression and signaling associate with the characteristic AMD symptoms, angiogenesis and leakage. In these species, angiogenic, but not normal vessels, selectively express the ROCK isoforms, qualifying them as biomarkers for AMD. Downstream mediators of ROCK—MYPT1, IκBa, and NF-κB—are activated in CNV, indicating a key role for this pathway in AMD. ROCK2 inhibition significantly reduces CNV and MYPT1 phosphorylation, while CNV is unaffected with endothelium specific deletion of ROCK1.

MaDAMs associate with human AMD, but not normal tissues. Analogous to tumor-associated macrophages (TAMs) that correlate with poor prognosis in cancer⁷, MaDAMs might predict AMD prognosis, or the risk of angiogenic proliferation. MaDAMs express both ROCK isoforms and ROCK2 inhibition reduces their number in CNV, without affecting total macrophage numbers. This preserves the beneficial macrophages that are necessary for ocular health⁶, and simultaneously restores the balance between proangiogenic MaDAMs and their angiostatic counterparts.

Age aggravates ROCK signaling and the M2-type immune response, evidenced by elevated pMYPT-1, pIκBa, CCR3, and IL-4. In contrast, age leaves the M1-type response unaffected. This establishes a novel link between age and ROCK-mediated macrophage differentiation. MCP-1−/− mice show pronounced ROCK signaling and an M2 shift with age. Interestingly, MCP-1−/− mice express the specific M2 marker CD163 in normal young and at higher levels in aged eyes, while normal WT eyes do not express comparable amounts of CD163. This indicates the ROCK-mediated M2-shift as a possible cause of the spontaneous proliferative changes in these animals.

ROCK signaling is identified herein as a regulator of macrophage differentiation. Aging stresses ROCK signaling, resulting into overt expression of the pro-angiogenic MaDAMs. This newly revealed chain of events can explain the proposed angiogenic switch in the aging eye⁵. Despite a high demand⁴, there has not been a specific ROCK2 inhibitor. Fasudil and Y27632 inhibit both ROCK isoforms, but also affect protein kinase A and C³. The antagonistic role of ROCK 1 and 2 in macrophage polarization indicates more efficacy and fewer side effects with specific inhibition of one isoform.

Described herein is the use of the orally available ROCK2 inhibitor, SLx2119, which has successfully completed a phase I clinical toxicity trial. Compared to anti-VEGF agents that treat late symptoms of proliferative AMD, targeting ROCK-2 and macrophage differentiation addresses earlier and mechanistic causes of AMD. The results described herein illuminate a to date unrecognized immune-modulatory root of AMD. NF-κB signaling was proposed in CNV² and maintenance of the M2 phenotype¹. It is demonstrated herein, however, that a suppressed endothelial NF-κB signaling, as in the Tie-1-ΔN mice, does not affect CNV. This and the presence of antiinflammatory M2-like macrophages in the human CNV lesions challenge the conventional paradigm that AMD is predominantly inflammatory. Instead, the present results indicate AMD's genesis in an immune imbalance. These insights permit new immune-based therapy of macular degeneration or other age-related diseases.

The work presented herein reveals the key role of ROCK-1 and -2 in macrophage polarization and remodeling of the eye microenvironment. Age and ROCK2 signaling determine the MaDAM phenotype, which drives the pathology.

### Example 3: ROCK-Isoform Specific Polarization of Age-Related Macular Degeneration Associated Macrophages: Introducing MaDAMs as a Cause of Disease

A mechanistic link between age and macular degeneration has not yet been established. Described herein is a novel regulatory role for the rho-associated kinase (ROCK) in both macrophage polarization and aging leading to age-related macular degeneration (AMD). A distinct M2-like macrophage population, the Macular Degeneration Associated Macrophages (MaDAMs), that expresses both ROCK isoforms was found in experimental and surgically removed human choroidalc neovascular (CNV) membranes, a main feature of AMD. ROCK1 inhibition increased M2-specific characteristics. In contrast, ROCK2 inhibition increased M1 macrophage markers and decreased MaDAMs and CNV pathology. In addition, intravitreal injection of exogenous M2 macrophages increased CNV via ROCK signaling, while M1 macrophages reduced lesions, indicating the causal role of M2 macrophages in AMD pathology.

M1 prone adiponectin−/− mice showed both, partial protection against laser-induced CNV, as well as no additional reduction of lesion size with ROCK2 inhibition. In contrast, M2 slanted IL-12p40−/− mice showed larger CNV lesions than WT controls. In WT and MCP-1−/− mice, age increased ROCK signaling and M2 characteristics, while M1 markers remained unchanged. This work reveals ROCK isoforms as a master switch in determining macrophage fate. Demonstrated herein is a connection between age and ROCK signaling as a cause of M2-like macrophage differentiation in CNV pathology.

Introduction.

Age-related macular degeneration (AMD) is the leading cause of adult vision loss. Most AMD cases are of the central geographic atrophy type, which at any time can become proliferative due to choroidal neovascularization (CNV). The normal choroid maintains quiescence through an excess of anti-versus pro-angiogenic factors. Little is known about what might reverse that balance and lead to an angiogenic switch. Aging is the strongest risk factor for AMD 1,2, however, how age is mechanistically linked to the pathogenesis is unknown. Polymorphisms in the complement factor H gene are strongly correlated with AMD 3-6, while the underlying patho-mechanisms remain to be investigated.

Vascular endothelial growth factor A (VEGF-A) is key to angiogenesis, but also fulfills important physiological functions in the retina 7. Current drug therapy in AMD is based on repeated intravitreal injections of VEGF-A inhibitors 8, which is not free of risks to the retina 7,9. A multi-center cohort study showed macular atrophy in virtually all longterm treated AMD cases, one third of which suffered an alarming visual decay 10.

Macrophages are critical components of AMD 11. In mice, deletion of the monocyte chemotactic protein-1 (MCP-1) causes AMD-like symptoms with aging 12, the mechanism of which is not understood. Undifferentiated M0 macrophages can polarize into the classical pro-inflammatory M1 and the alternative anti-inflammatory M2 13, both of which are found in AMD 14. Depending on the microenvironment, macrophages differentiate into either phenotype 13. IL-1β, IL-12, IL-23, IFN-γ, LPS, and TNF-α induce the M1-like phenotype that expresses CCL3, CCL5, CD80, CCR7, iNOS, and INFγ 13. In contrast, IL-4, IL-10, IL-13, and TGF-β promote the M2-like phenotype that expresses CCL22, CD206, CD163, YM 1, Fizz 1, and arginase 1 13. Known regulators of macrophage polarization include the Krüppel-like factor 4 15 and adiponectin 16. Adiponectin promotes M2 cells, and adiponectin-deficient mice express more M1 cells 16.

Rho-associated, coiled-coil-containing protein kinases (ROCKs) are involved in cytoskeletal rearrangement, contractility 17, angiogenesis 18 and inflammation 19. ROCK inhibition increases the survival of human embryonic stem cells 20. Upstream of ROCK are RhoA and RhoE which are involved in cytoskletal functions. ROCK has two isoforms, ROCK1 and ROCK2, whose intracellular localizations widely differ depending on the type and condition of the examined cells 21,22. The distribution of ROCK isoforms in macrophages is not known. Similarly, the role of ROCK signaling in macrophage polarization and AMD has not been explored. Immediate ROCK substrates are myosin light chain (MLC), myosin binding subunit of myosin phosphatase (MYPT), and ezrin/radixin/moesin (ERM) proteins 23, while downstream targets include IκB-α and NF-κB 24. ROCK phosphorylates MLC phosphatase, causing smooth muscle contraction and vasoconstriction 25.

The work presented herein describes a previously unknown molecular switch for macrophage polarization through the ROCK signaling pathway. An M2 shift with physiologic aging and the causal role of M2-like macrophages in CNV pathology are established herein.

### Results

Choroidal Neovascular Endothelium Expresses ROCK.

To determine if ROCK1 and ROCK2 are expressed in human AMD, surgically excised CNV membranes from AMD patients were used for immunohistochemistry (data not shown). In these samples staining was done for both ROCK isoforms and for the von Willebrand Factor (vWF). Surgically excised CNV membranes from human AMD (n=7) and idiopathic macular degeneration (n=7) patients expressed both ROCK1 and ROCK2 (14 out of 14). Sections from two different AMD membranes revealed excess extracellular matrix, stromal cells, pigmented epithelial cells and neovascularization that is characteristic of proliferative disease.

In contrast, in none of the normal patient specimens there was staining for either isoform (0 out of 5). Analogously, in the laser-induced CNV in mouse and monkey, ROCK1 and ROCK2 co-localized in angiogenic vWF-positive endothelium, but were not detected in the normal vessels (data not shown). In the CNV tissues of all three species, non-endothelial cells also expressed ROCK isoforms.

Increased ROCK Signaling in Choroidal Neovascularization

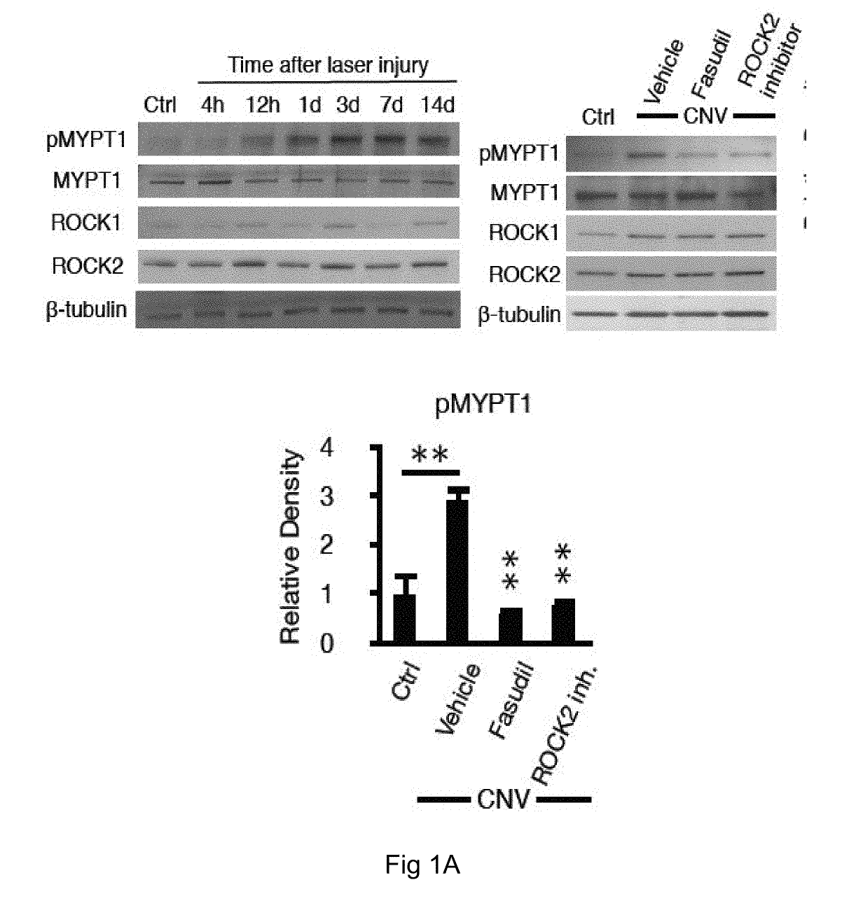

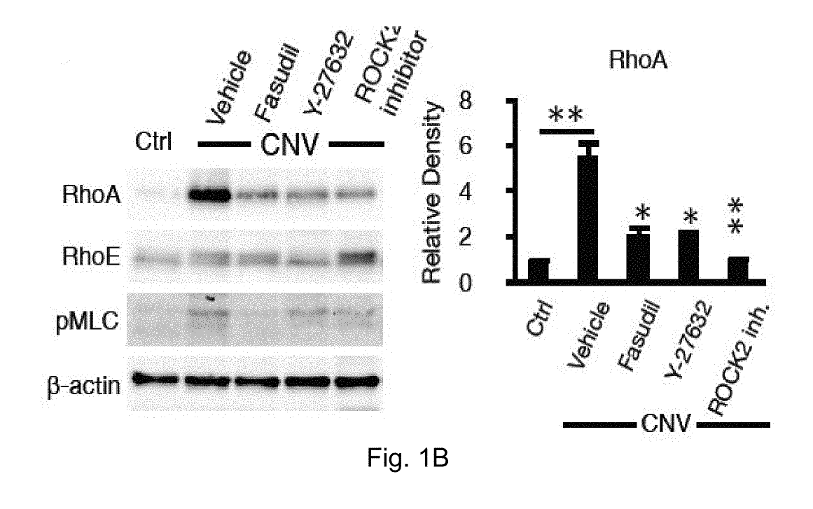

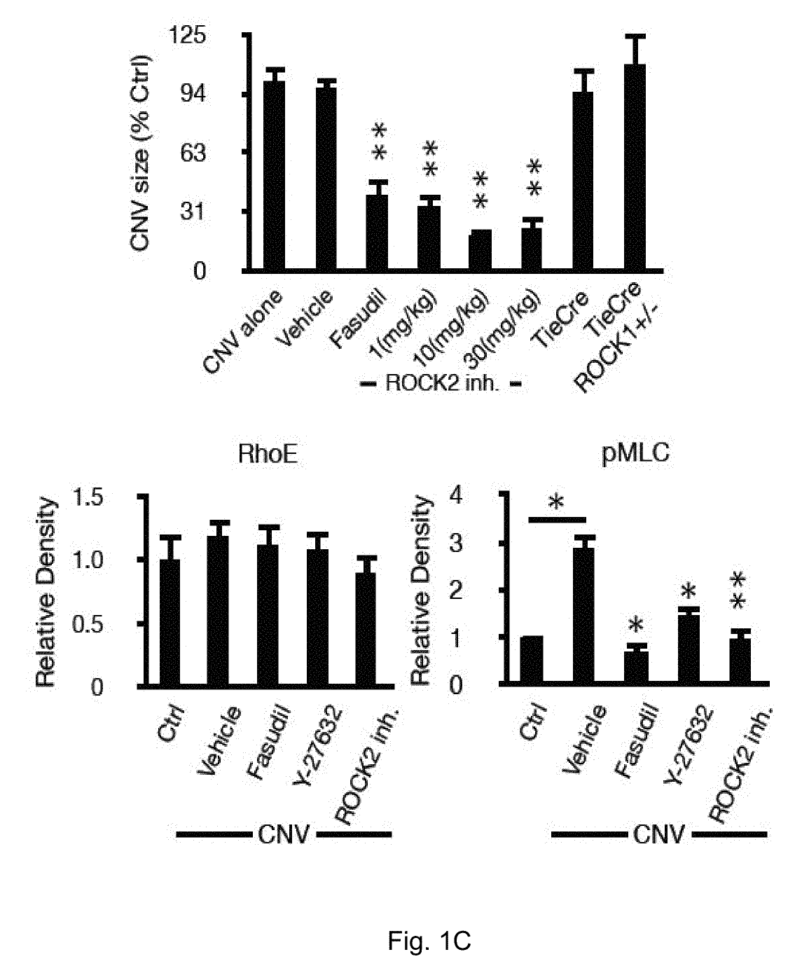

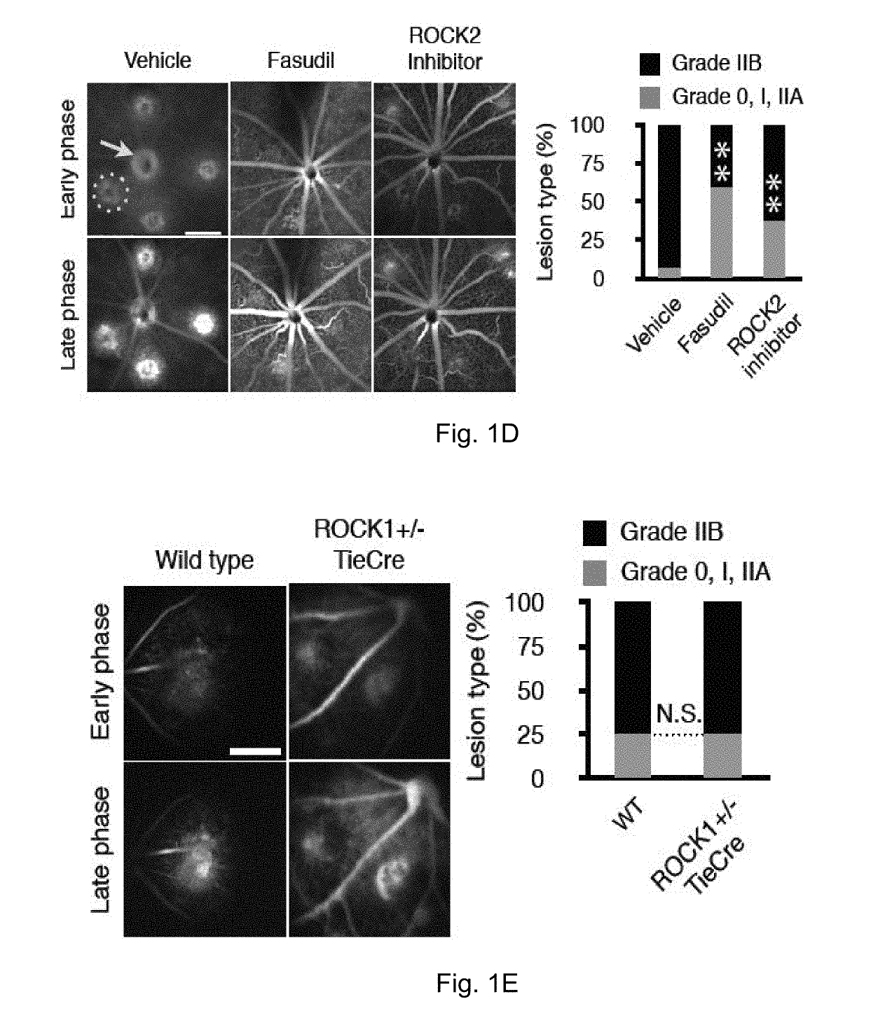

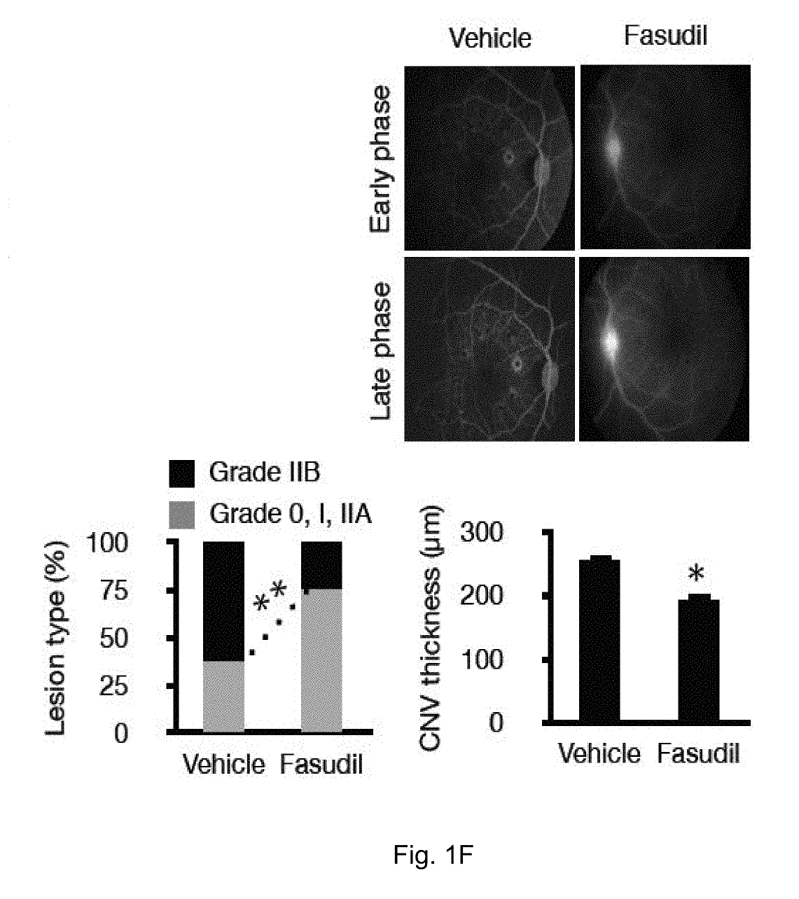

To investigate ROCK signaling in CNV, western blots were performed for ROCK isoforms and one of its substrates, MYPT1. MYPT1 phosphorylation peaked at 3 and 7 days following laser injury in the mouse choroid, while ROCK1 and ROCK2 expressions remained unchanged throughout the course of CNV. Pan ROCK (both ROCK isoforms) and selective ROCK2 inhibition reduced pMYPT1 to normal unlasered levels (FIG. 1A). MLC phosphorylation was increased in CNV and reduced to normal levels with pan ROCK and selective ROCK2 inhibition. Upstream of ROCK, RhoA was significantly increased in CNV, while RhoE remained unchanged. Pan ROCK and selective ROCK2 inhibition significantly reduced RhoA expression in lasered eyes to those found in unlasered controls (FIG. 1B).

ROCK2 Inhibition Reduces Choroidal Neovascularization and Leakage

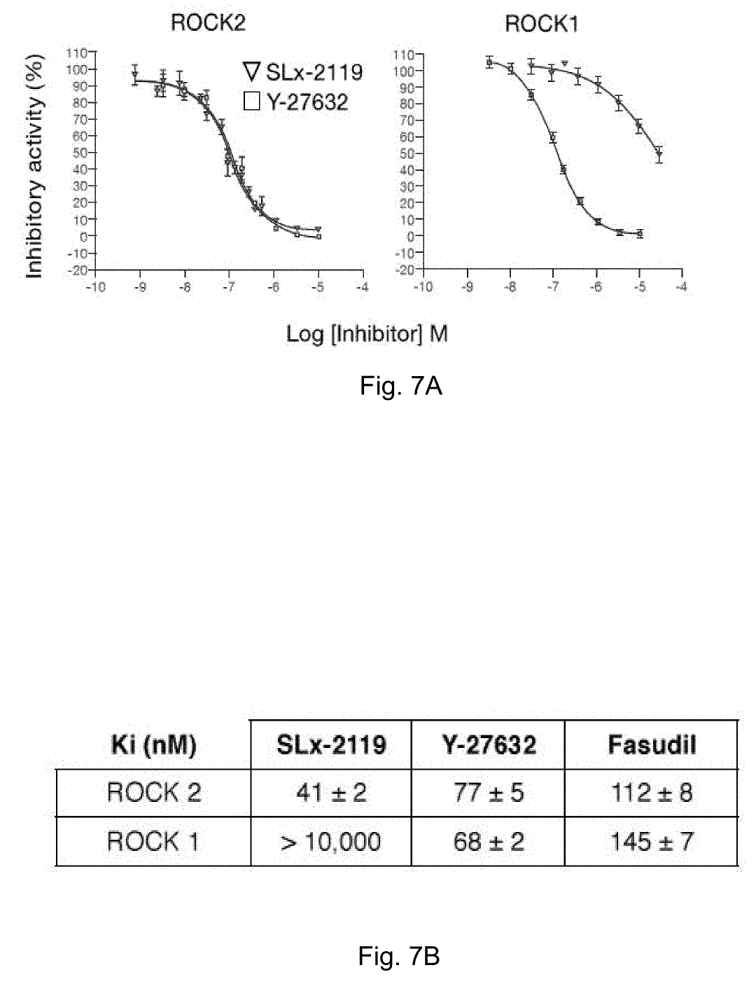

To investigate the contribution of ROCK signaling to CNV formation, CNV was induced by laser injury and the animals treated with the pan ROCK inhibitor, fasudil, or the selective ROCK2 inhibitor. Lesion size and vascular leakage were then quantified. In rodents, CNV lesions were significantly smaller with pan ROCK inhibition compared to control, showing an approximate 60% reduction. To block ROCK2 activity we used an isoform specific inhibitor (FIGS. 7A-7D). Intraperitoneal injection of the selective ROCK2 inhibitor significantly reduced CNV in a dose-dependent fashion. At a dose of 1 mg/kg, it reduced CNV by 66%, while maximum efficacy was reached at 10 mg/kg. In comparison, the CNV size in ROCK1+/−Tie1Cre mice that have reduced endothelial specific ROCK1, did not differ from that of lasered Tie1Cre or WT control (FIG. 1C).

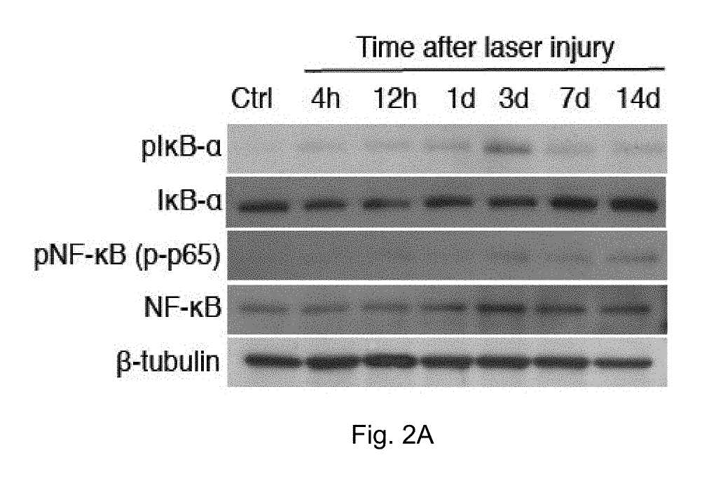

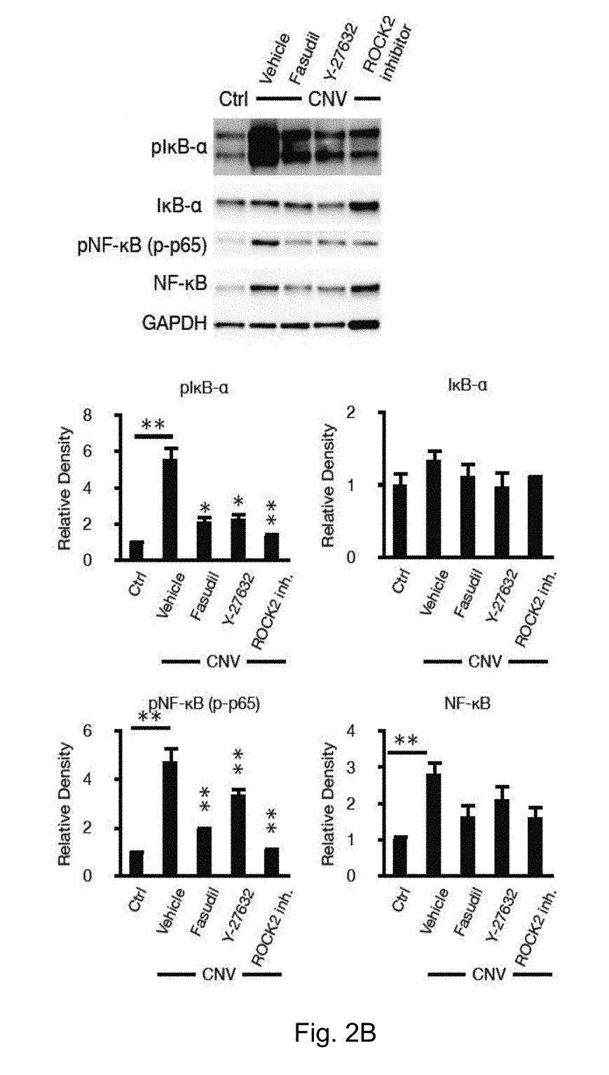

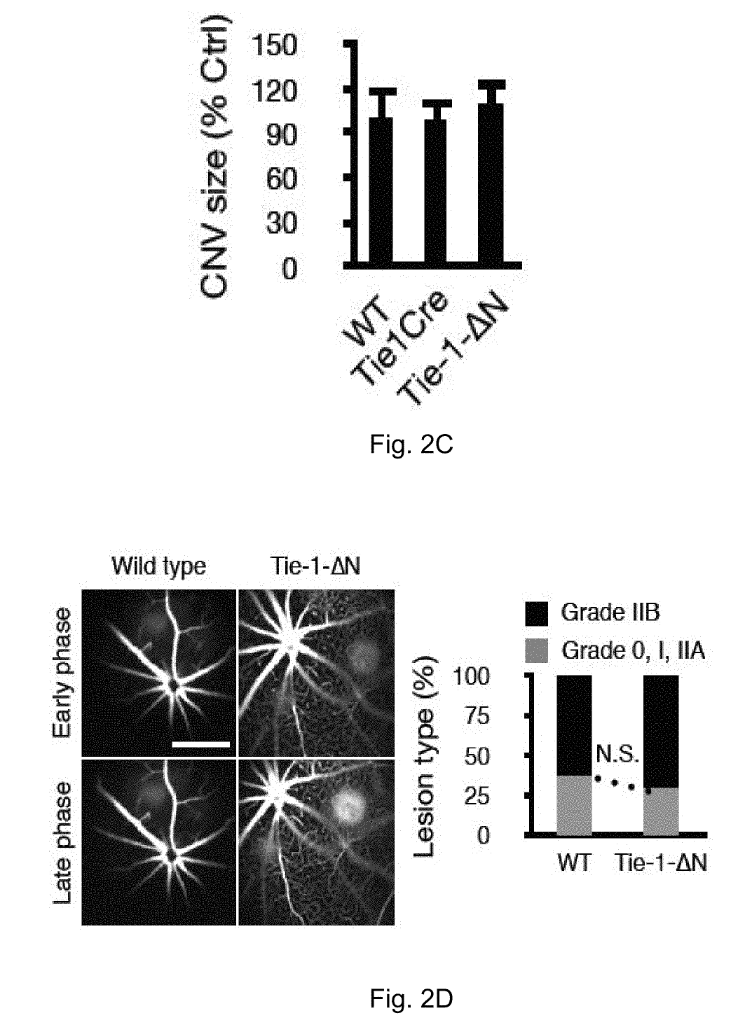

Pan ROCK and selective ROCK2 inhibition supressed the percentage of the clinically relevant 2B leakages in mice (FIG. 2D). In comparison, ROCK1+/−TieCre showed the same amount of leakage as lasered WT (FIG. 2E). Furthermore, intravitreal injections of fasudil in monkeys significantly reduced the percentage of the clinically relevant 2B leakages, as well as CNV membrane thickness (FIG. 2F).

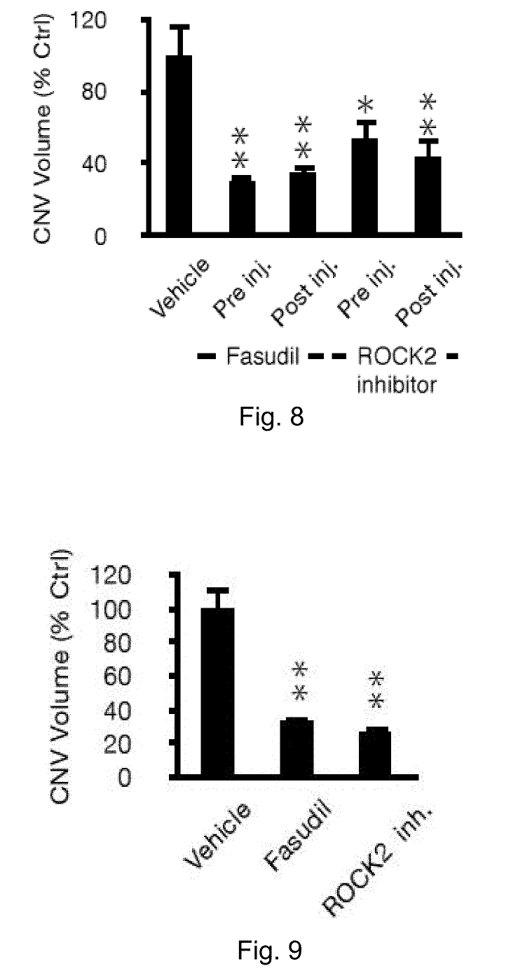

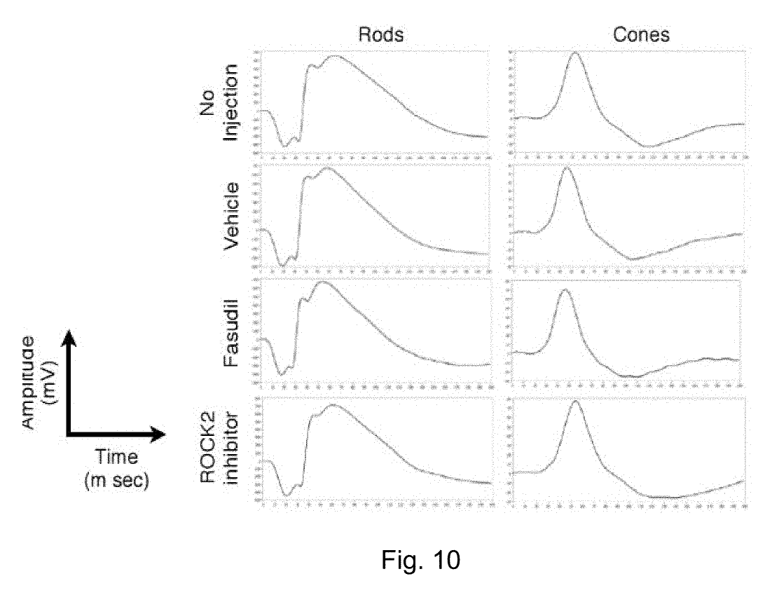

Pan ROCK and ROCK2 inhibitor treatments, starting one week after CNV formation, reduced CNV lesions on day 14, which was comparable to the outcome of early treatments that started on day 1 (FIG. 8). Since intravitreal injections of VEGF-A inhibitors are the current standard in AMD treatment, the effect of ROCK inhibitors on CNV formation and retinal toxicity was tested. Intravitreal ROCK2 or pan ROCK inhibition reduced CNV size (FIG. 9), but did not show changes in the electroretinogram (ERG) (FIG. 10).

Endothelial NF-κB not Required for CNV Formation

To study downstream mediators of ROCK, IκB-α and pIκB-α was measured in the eyes of normal and lasered animals with and without inhibitor treatments. IκB-α phosphorylation peaked 3 days after laser injury, while phosphorylated NF-κB p65 (RelA) was detectable by day 3 through 14 (FIG. 2A). Pan ROCK and selective ROCK2 inhibition reduced CNV-induced IκB-α and NFκB phosphorylations. The IκB-α protein expression was not affected by laser injury or the various treatments (FIG. 2B).

The implication of NF-κB in angiogenesis 26, motivated the examination of the role of endothelial NF-κB signaling in CNV. The Tie IAN mouse, which was generated by crossing the foxed IκB-αΔN (loxP-ΔN) with the Tie1Cre knock-in mouse 27 was studied. In these mice, CNV and leakage was the same as that in lasered WT or in the Tie1Cre control mice (FIGS. 2C-2D).

ROCK Mediated Macrophage Infiltration in Choroidal Neovascularization

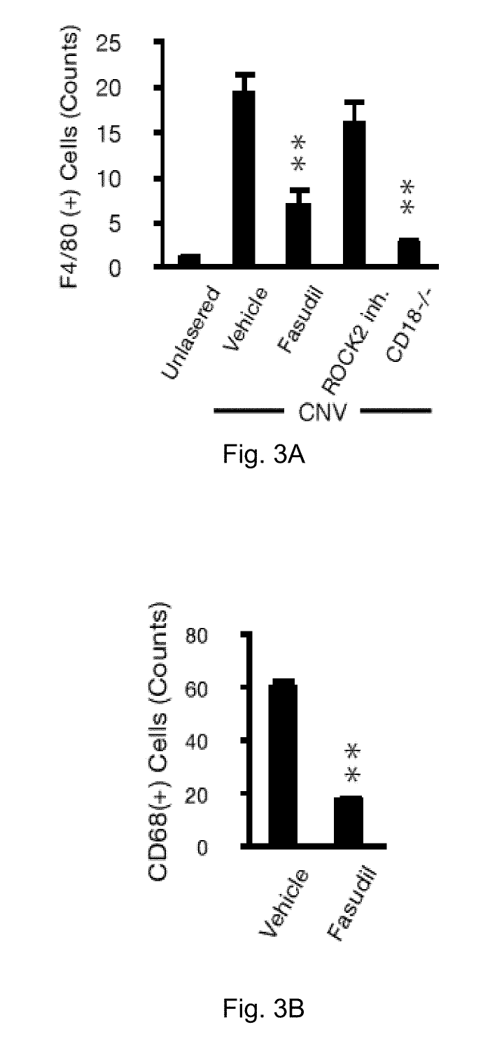

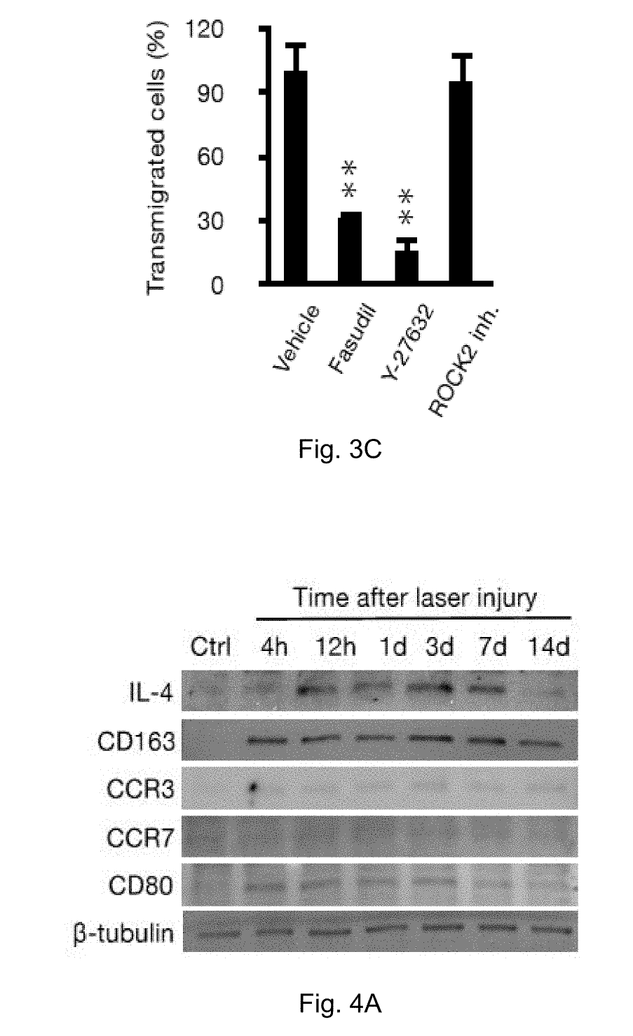

To investigate the role of ROCK in macrophage infiltration, staining was performed for the macrophage marker F4/80 in CNV. Macrophage recruitment peaks 3 days after laser injury 28. The number of accumulated F4/80(+) macrophages at day 3 was significantly reduced by pan ROCK, but not by selective ROCK2 inhibition (FIG. 3A). As a control, lasered CD18−/− mice with a known leukocyte recruitment deficiency showed levels of F4/80(+) cells comparable to that of unlasered WT mice. In monkey eyes, intravitreal fasudil injection significantly reduced macrophage infiltration into CNV lesions (FIG. 3B). Immunohistochemistry showed staining for both ROCK isoforms in infiltrated macrophages in the CNV lesions of mouse and monkey, but not in normal control eyes (data not shown).

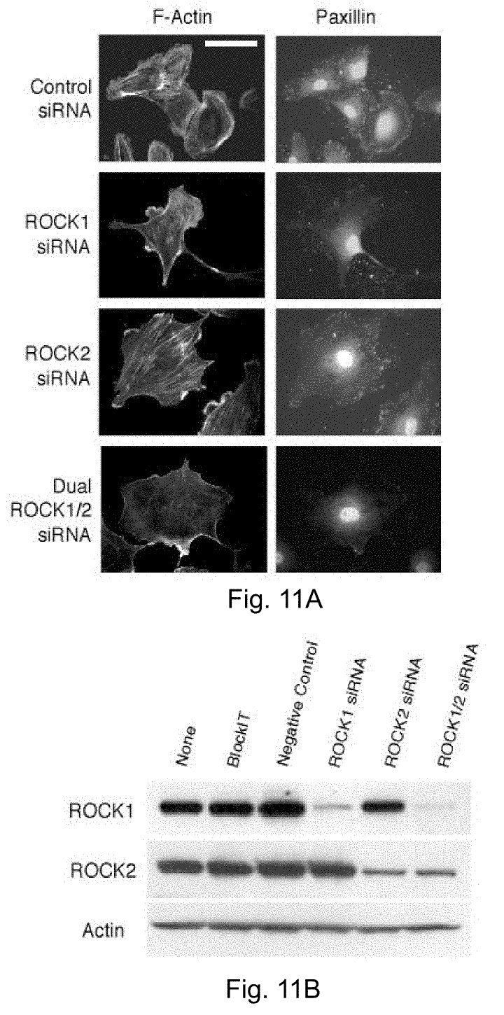

To investigate the differential impacts of pan ROCK versus ROCK2 inhibition on leukocyte transmigration from angiogenic vessels, a new imaging technique was developed (data not shown). By combining growth-factor-induced corneal angiogenesis with in vivo AO labeling of leukocytes, visualization of leukocyte extravasation from limbal and angiogenic blood vessels in a chemotactic gradient was achieved. In MCP-1 implanted corneas, pan ROCK inhibition suppressed leukocyte transmigration, while selective ROCK2 inhibition did not (FIG. 3C). This is in line with the finding that pan ROCK inhibition or ROCK1 knockdown affects cytoskeletal proteins, while selective ROCK2 inhibition or ROCK2 knockdown does not (FIGS. 11A-11B). The distribution of paxillin was also examined. In the vehicle treated controls, paxillin was distributed in the cytoplasm. When both ROCK isoforms were blocked, using the pan-ROCK inhibitors fasudil or Y-27632, paxillin was no longer found evenly spread in the cytoplasm. In these cells paxillin was concentrated in the nuclear or immediate perinuclear regions. In comparison, in the ROCK2 inhibitor treated cells, paxillin distribution in the cells was comparable to the control cells. These results confirm the impact of ROCK isoform knockdowns with siRNAs (data not shown).

ROCK Mediated Macrophage Polarization in Choroidal Neovascularization

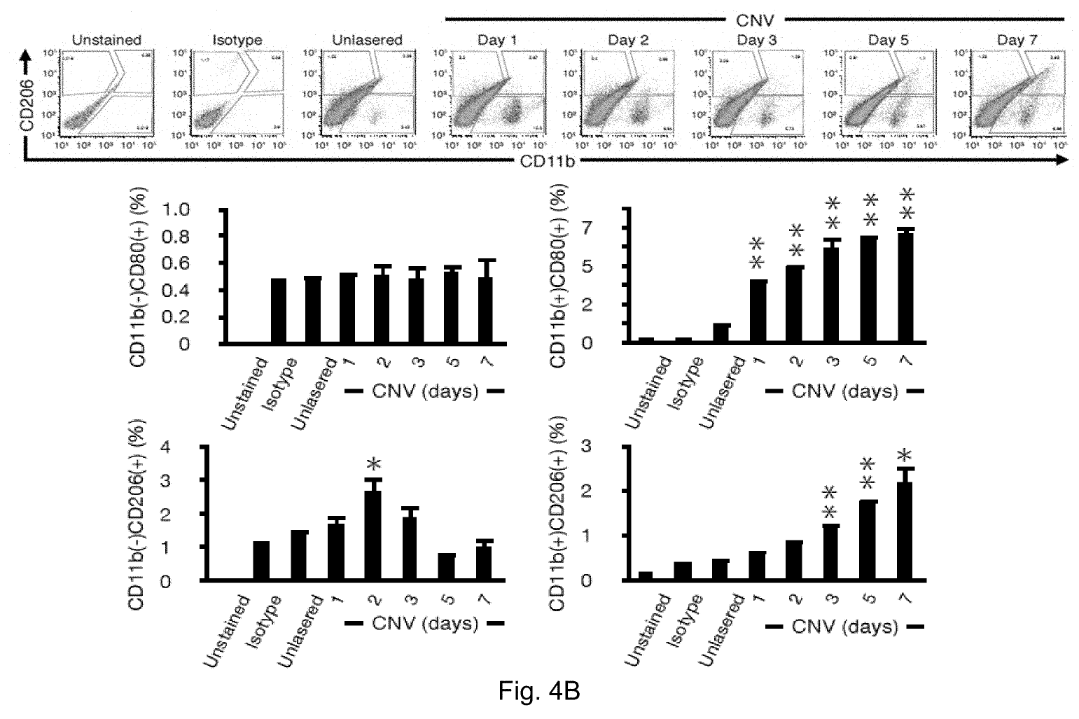

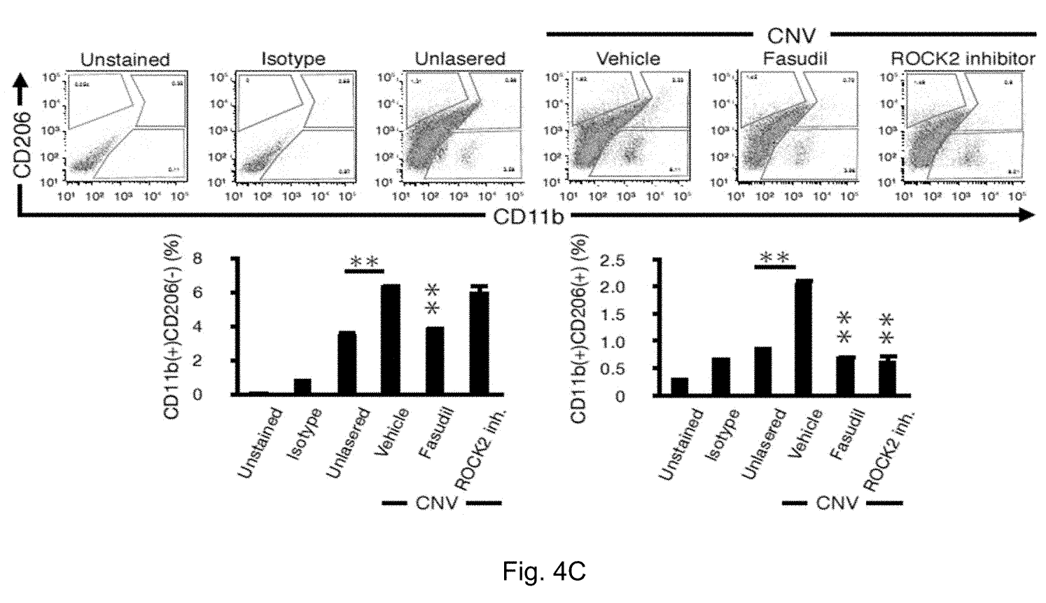

The role of ROCK isoforms in macrophage polarization has previously not been investigated. In surgically excised membranes from AMD patients, ROCK1 and ROCK2 co-localized with CD206, expressed on M2 macrophages (data not shown). CD80(+) cells expressed ROCK1 and ROCK2 in neovascular tissues from AMD patients, whereas normal eyes did not stain for CD80, CD206, or either ROCK isoform. The time course of protein expression showed elevated IL-4 and CD163 levels in CNV through day 7 or day 14, respectively. CCR7 was unchanged while CD80 was moderately higher in the first three days. CCR3 levels remained unchanged at the examined time points (FIG. 4A).

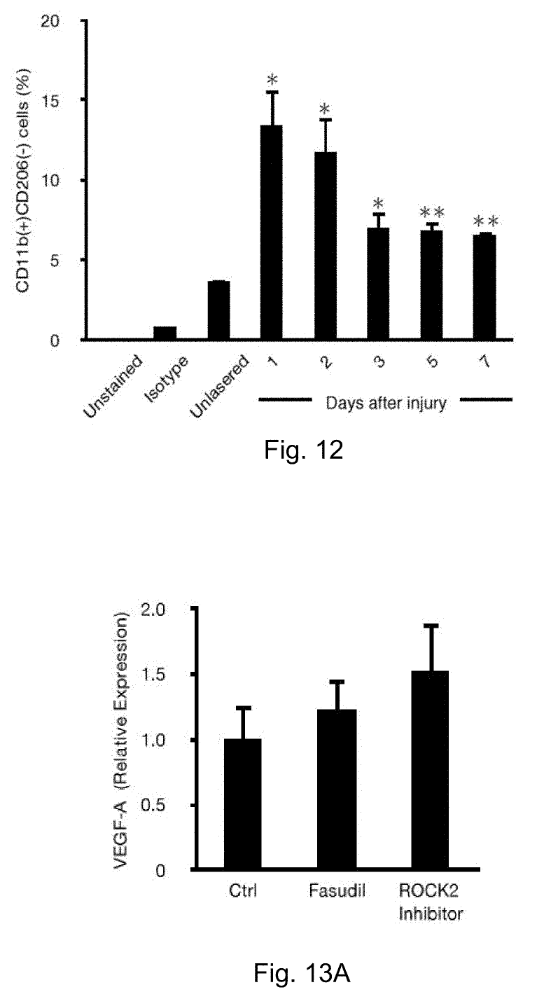

To further characterize macrophage phenotypes, flow cytometry was performed for M1- and M2-like macrophages in normal and lasered mouse eyes during CNV development. The number of CD11b(+)CD80(+) M1-like macrophages increased on day 1 after laser injury and remained high through day 7. In the CNV model, angiogenesis starts 3 days after laser injury and peaks on day 7 29. A peak of CD11b(−)CD206(+) cells was found on day 2 post laser injury, which preceded the reported start of angiogenesis. On days 3 through 7 the percentage of CD11b(+)CD206(+) cells increased with a peak on day 7, coinciding with the maximum angiogenic response in the laser-injury model (FIG. 4B). CD11b(+)CD206(−) peaked on day 1 after laser injury, which could be due to an initial neutrophil accumulation after laser injury (FIG. 12).

Pan ROCK and ROCK2 inhibition substantially decreased the CD11b(+)CD206(+) M2 population, when examined on day 7. The CD11b(+)CD206(−) cell population was reduced by pan ROCK inhibition but not by ROCK2 inhibition (FIG. 4C).

ROCK Regulates Macrophage Polarization

To investigate the role of ROCK signaling in macrophage polarization, staining for each isoform was performed in macrophages (data not shown). In undifferentiated M0 macrophages ROCK1 and ROCK2 were evenly distributed in the cytoplasm. In M1 macrophages, ROCK1 was concentrated in the pen-nuclear regions. In comparison, ROCK2 was distributed in the cytoplasm. Interestingly, the cytoplasmic distribution of ROCK2 in M1 macrophages showed unique circular areas of non-expression reminiscent of vacuoles. In M2 macrophages, ROCK1 was evenly distributed in the cytoplasm, while ROCK2 was highly concentrated near the nucleus. In a small number of M1 cells, ROCK2 was also found concentrated around the nucleus.

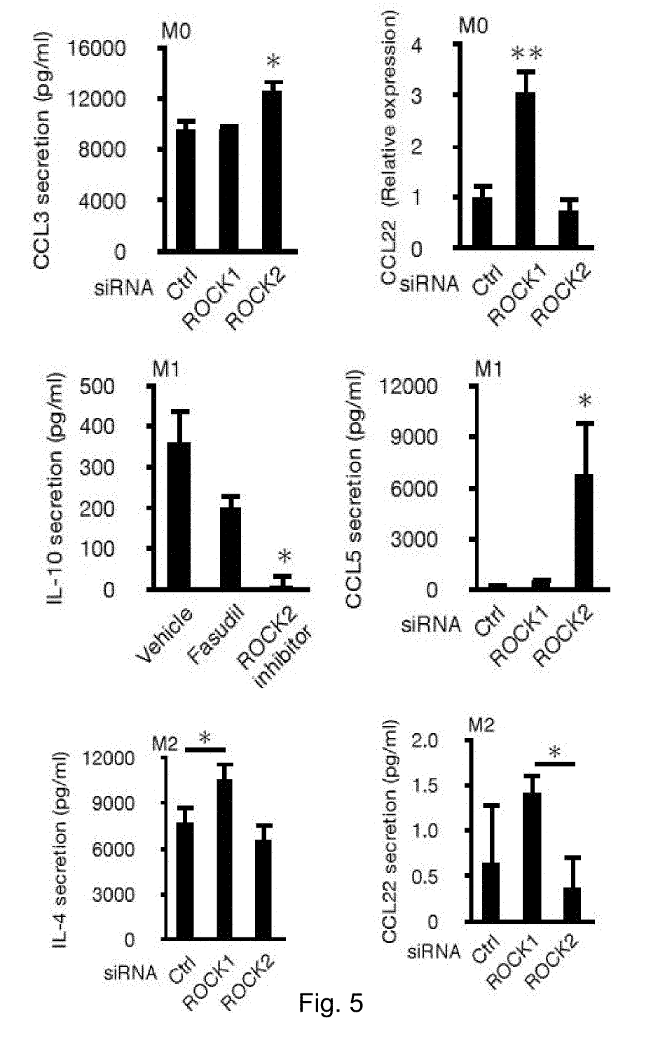

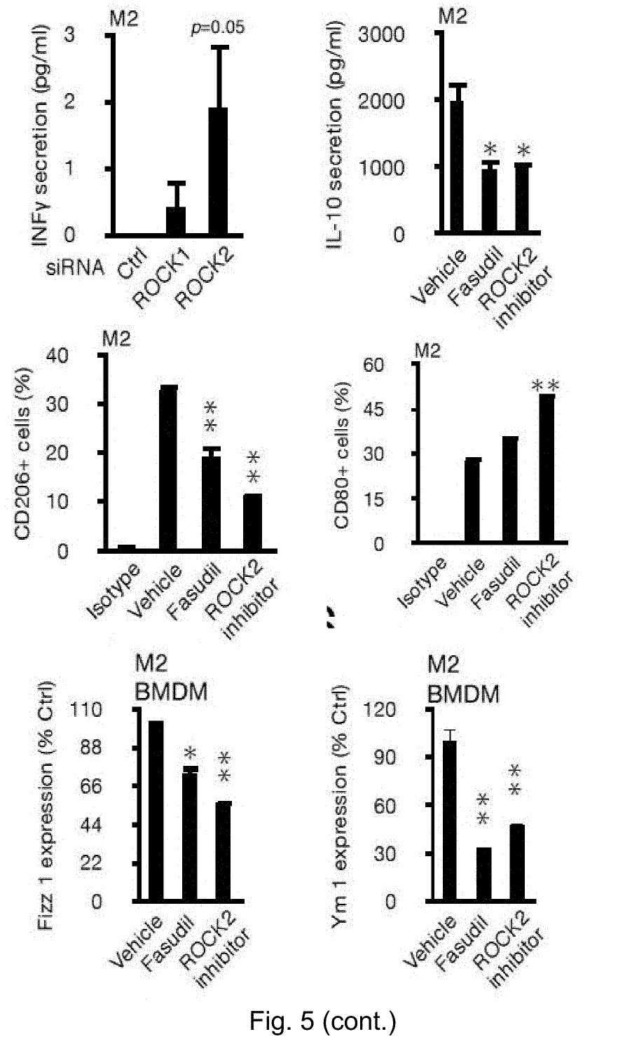

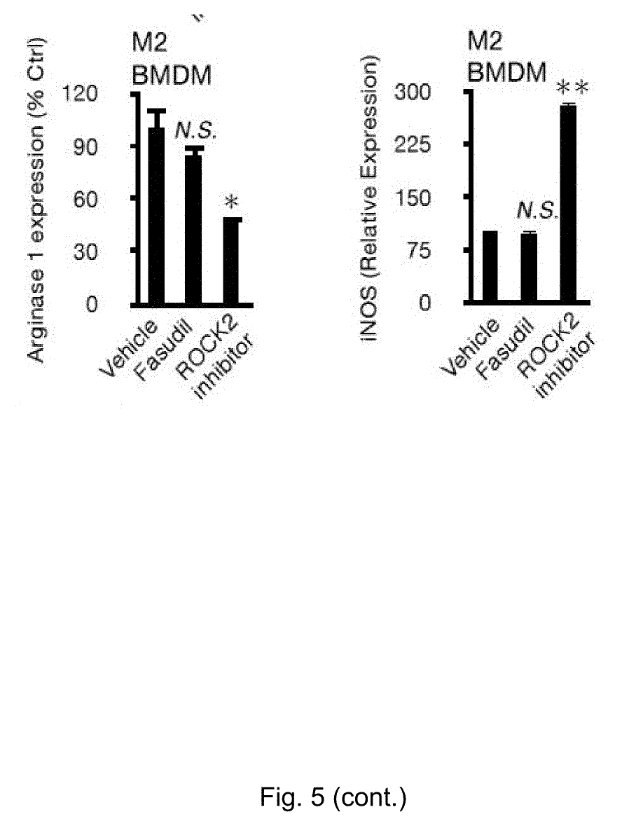

To elucidate the role of ROCK in macrophage fate, genetic knockdown and pharmacological inhibition was used (FIG. 5). In M0 cells ROCK2 knockdown increased CCL3, while ROCK1 knockdown increased CCL22. In an M1 environment, ROCK2 inhibition decreased IL-10 secretion, while it increased CCL5. In an M2 environment, ROCK1 knockdown increased IL-4 and CCL22 secretion, while ROCK1 knockdown increased IFN-γ secretion. Both pan ROCK and selective ROCK2 inhibition reduced IL-10 secretion. Furthermore, pan ROCK and selective ROCK2 inhibition reduced the percentage of CD206(+) cells in flow cytometry, while ROCK2 inhibition increased the percentage of CD80(+) cells.

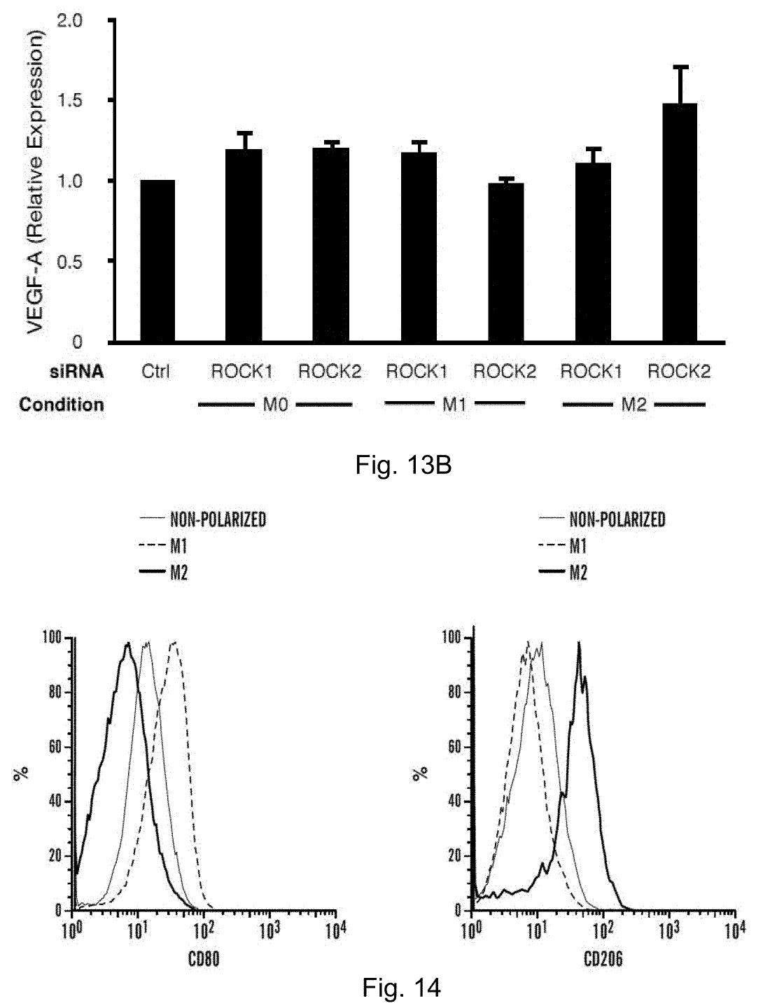

In bone marrow derived M2 macrophages, pan ROCK and selective ROCK2 inhibition reduced Fizz 1 and YM 1. In comparison, only ROCK2 inhibition but not pan ROCK inhibition reduced arginase 1. In contrast, ROCK2 inhibition significantly increased iNOS expression, as determined by RT-PCR. These data indicate a previously unknown regulatory role for ROCK isoforms in macrophage polarization. Pharmacologic inhibition of ROCK or knockdown of ROCK isoforms did not affect VEGF-A expression (FIGS. 13A-13B).

Casual Role of M2-Macrophages in Choroidal Neovascularization.

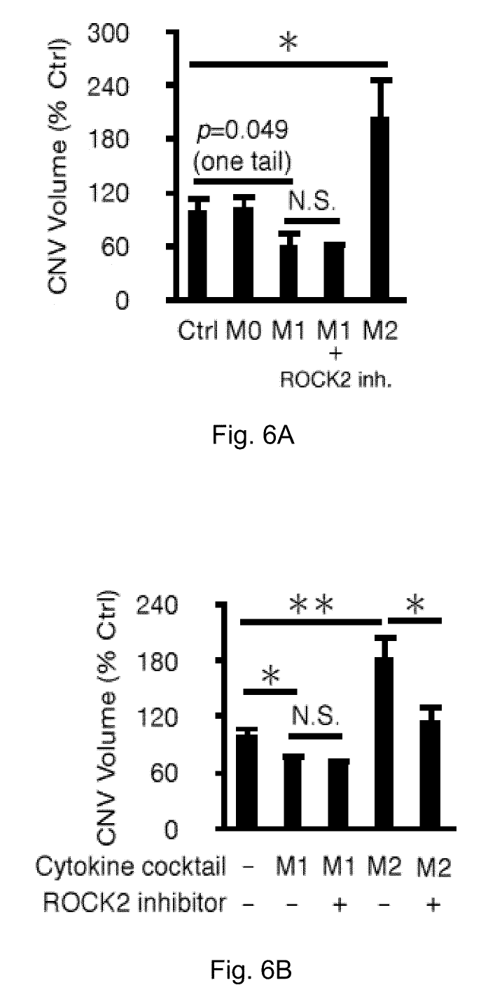

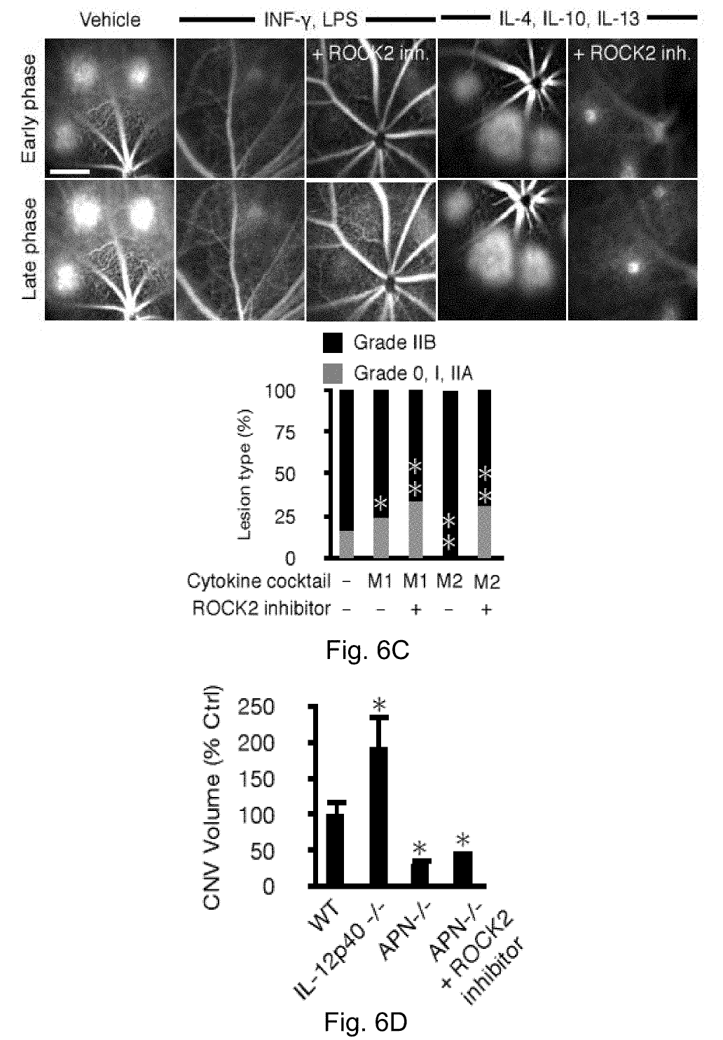

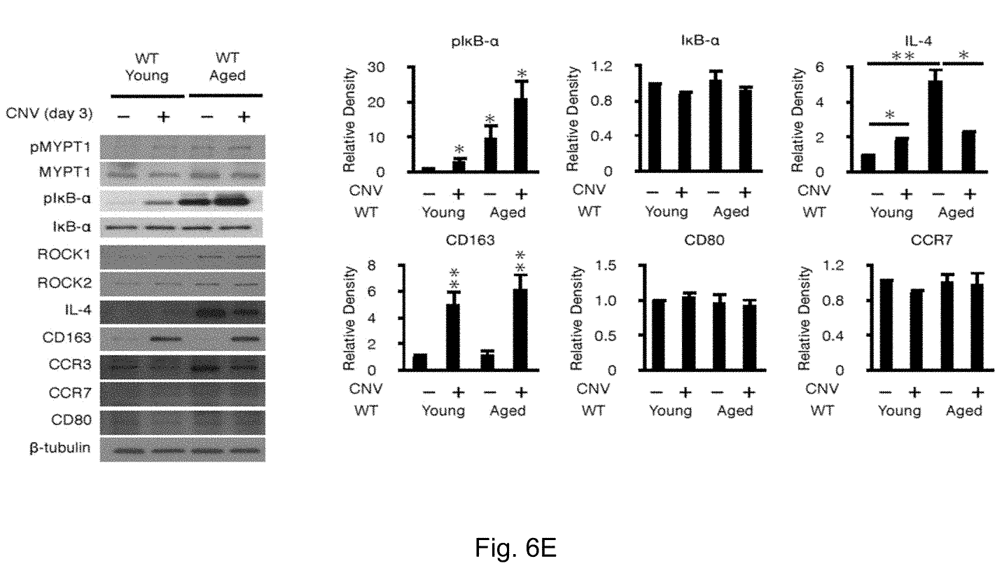

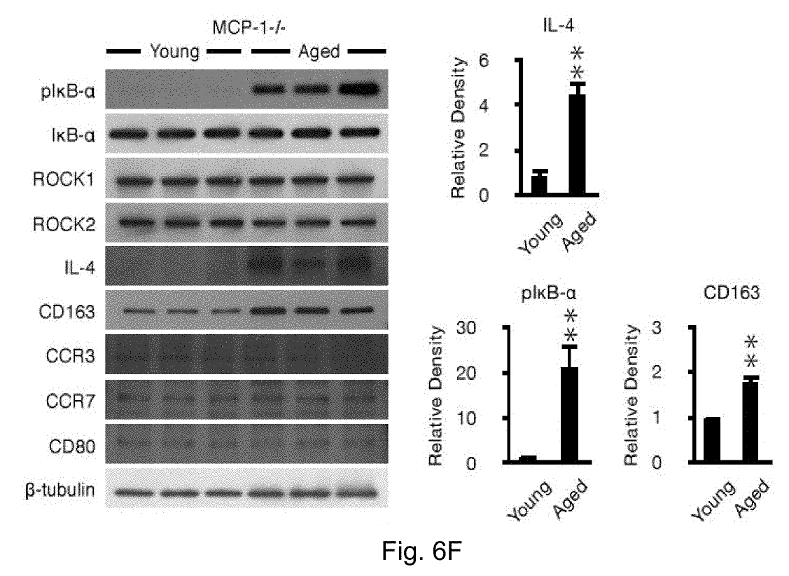

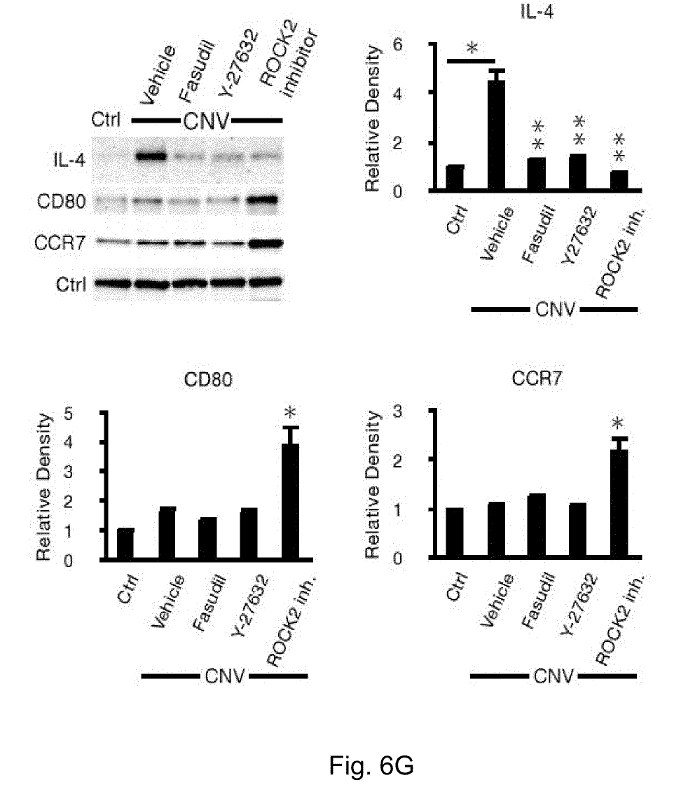

To examine the contribution of macrophage subtypes in CNV formation, murine bone marrow-derived macrophages were differentiated into M1 or M2 phenotype (FIG. 14) and injected into the vitreous of laser treated WT mice. While undifferentiated macrophages did not affect CNV, M2 macrophages increased the lesion size. In comparison, M1 macrophages reduced CNV lesions. Intravitreal injection of M1 macrophages in lasered mice that were treated with the ROCK2 inhibitor did not reduce lesion size any further, indicating that the beneficial effect of ROCK2 inhibition in vivo is indeed through macrophage polarization (FIG. 6A).

Next, WT animals were intravitreally injected with M1 (INF-γ and LPS) or M2 (IL-4, IL-10, and IL-13) transforming cytokine cocktails. Intravitreal injection of INF-γ and LPS reduced CNV size and leakage, in line with the previous report that low dose systemic LPS reduces CNV 30. CNV and leakage in the M1-cytokine cocktail injected eyes remained unaffected when the animals were in addition ROCK2 inhibitor treated. In contrast, intravitreal injection of IL-4, IL-10, and IL-13 increased CNV size and the percentage of clinically relevant 2B leakage, which were reduced to control levels when the animals were in addition ROCK2 inhibitor treated (FIGS. 6B-6C). These results indicate the importance of the retinal cytokine profile in CNV, mediated through ROCK signaling.

M2 prone IL-12p40−/− mice had larger CNV lesions than WT animals. In contrast, M1 slanted adiponectin−/− mice had smaller CNV lesions than WT. These results indicate that an endogenous bias for macrophage subtypes affects CNV pathology. Also, ROCK2 inhibition did not further decrease CNV size in adiponectin−/−mice (FIG. 6D). The lack of efficacy of ROCK2 inhibition in these mice further supports the finding that the beneficial effects of ROCK2 inhibition are through a shift in macrophage polarization.

Age Compounded ROCK Signaling and an M2 Shift

To investigate ROCK signaling as a function of age and disease, ROCK isoforms, their downstream mediators, and M1 and M2 markers were quantified in the choroids of young (8-12 week old) and aged (>16 month old) WT mice with and without CNV. Compared to the baseline in the normal young, IκB-α phosphorylation was higher in the lasered young and in the normal aged animals, with the highest levels found in the aged lasered animals, suggesting a compounding effect of age. In comparison, IκB-α protein expression did not differ between the groups (FIG. 6E).

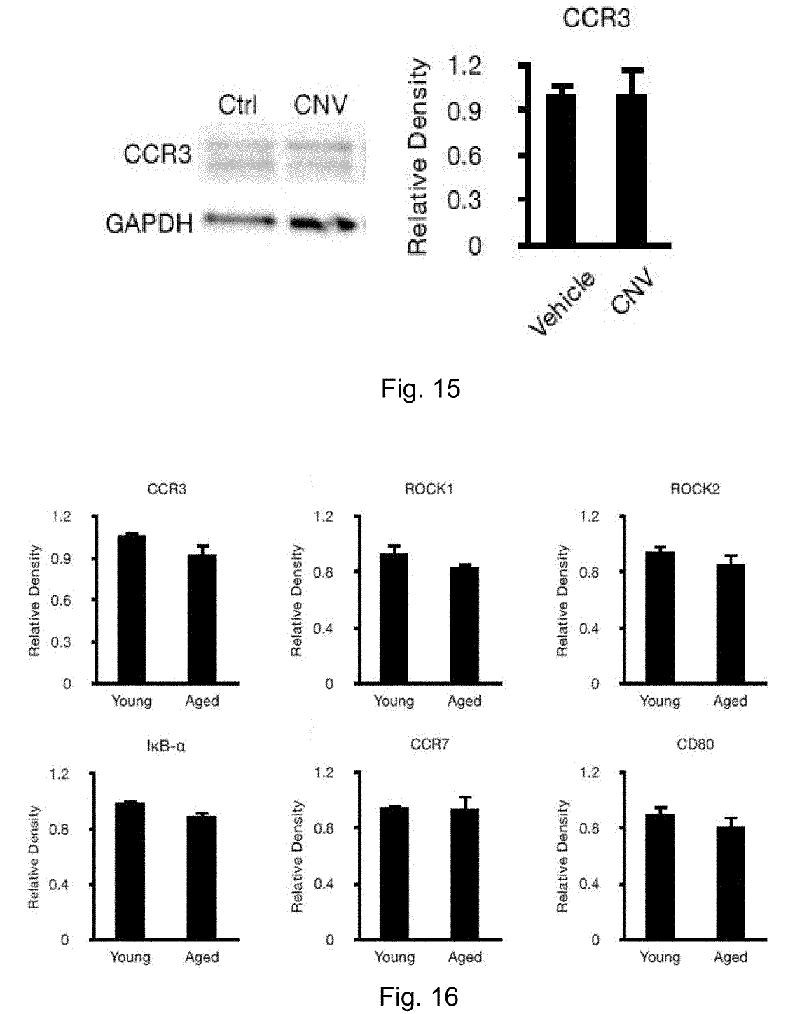

IL-4 was elevated in the young WT animals with CNV compared to the base levels in the normal young eyes. Unlasered aged WT mice had higher IL-4 levels compared to young WT animals even with CNV. Interestingly, the highest IL-4 level was found in the unlasered aged animals. CD163 was higher in CNV, although it did not change with age. The M1 specific markers CCR7 and CD80 were at similar levels in young and aged animals with or without laser injury (FIG. 6E). Interestingly, CCR3 that was previously reported to be upregulated in CNV 31, was unchanged in choroidal tissues of lasered mice (FIG. 15).

Aged MCP-1−/− mice spontaneously develop CNV, rendering them a realistic model of AMD 12. However, the cause of proliferation in these animals is unknown. Significantly higher IL-4 was found in aged MCP-1−/− mice, that could underlie an M2 shift. Surprisingly, CD163 was found in normal unlasered young MCP-1−/− mice, which further increased in aged MCP-1−/− mice (FIG. 6F)). CD163(+) cells were found in CNV lesions of WT mice and the posterior segment of aged MCP-1−/− mice (data not shown). IκB-α phosphorylation was substantially upregulated in aged MCP-1−/− mice (FIG. 6F). In contrast, M1-specific markers, CCR7 and CD80 remained unchanged in aged MCP-1−/− mice, as compared to those of young animals (FIG. 16). The increased IL-4 in CNV was reversed by pan ROCK or selective ROCK2 inhibition. Strikingly, ROCK2 inhibition but not pan ROCK inhibition increased the M1 markers CD80 and CCR7 in lasered eyes (FIG. 6G).

### Discussion

ROCK signaling is demonstrated herein to be a master switch in macrophage polarization and the M2-like macular-degeneration-associated macrophages (MaDAMs) are identified as a cause of disease. As a source of VEGF-A, macrophages promote CNV 11. Paradoxically however, macrophages can also inhibit CNV 28. The work described herein resolves this apparent discrepancy and provides novel mechanistic insights into how macrophages decrease and increase CNV, depending on their phenotype. M1 macrophages reduce, while M2 macrophages increase CNV. It is demonstrated herein that ROCK signaling determines macrophage fate. ROCK2 inhibition suppresses M2 differentiation, while it furthers M1 polarization, the latter, even in an M2 cytokine environment. In contrast, ROCK1 inhibition furthers M2 polarization.

A mechanistic distinction is that ROCK1 inhibition reduces macrophage extravasation while ROCK2 inhibition does not affect it. The partial protection against laser-injury in the M1 prone adiponectin−/− mice further strengthens the key role for macrophage polarization in CNV. The adiponectin receptor 1 is implicated in human AMD 32, however the mechanistic links remain to be investigated. Furthermore, the lack of function of the ROCK2 inhibitor in the adiponectin−/− mice suggests that the beneficial effects of selective ROCK2 inhibition are through polarization from M2 to M1, which in these mice is likely to be ROCK unrelated. In contrast, the M2 prone IL-12p40−/− mice show a larger CNV than WT.

In mouse, monkey, and human, ROCK expression and signaling are associated with CNV. In AMD membranes, angiogenic but not normal vessels express the ROCK isoforms, making them biomarker candidates. Phosphorylation of downstream mediators of ROCK—MLC, MYPT1, IκB-α, and NF-κB p65—are increased in CNV and suppressed with pan ROCK or selective ROCK2 inhibition, suggesting a key role for this pathway in CNV. Pan ROCK and selective ROCK2 inhibition significantly reduce CNV, while reduced endothelial-specific ROCK1 does not. Upstream of ROCK, RhoA is increased in CNV and reduced to normal levels with ROCK inhibition. RhoA activation causes translocation of ROCK2 33, which is in line with the cytosolic changes found in M2 macrophages.

MaDAMs are found in AMD membranes, but not in the normal retina. Their accumulation could be due to changes in the fundus microenvironment. For instance, in CNV and in aged unlasered animals elevated IL-4 was found. Interestingly, only M2 macrophages and not M0 or M1, secrete IL-4. This is in line with the fact that lung tissue macrophages secrete IL-4, while monocytes do not 34. In CNV, pan ROCK and ROCK2 inhibition reduce IL-4 to baseline levels. In addition to shifting the macrophage balance from M2 to M1, ROCK2 inhibition also affects the cytokine micro-environment in which macrophages differentiate.

In analogy to tumor-associated macrophages that correlate with poor prognosis in cancer 35, MaDAMs could indicate the onset or severity of AMD, for instance if quantified by molecular imaging 36. MaDAMs express both ROCK isoforms and ROCK2 inhibition reduces their number in CNV, without affecting total macrophage numbers. ROCK2 but not pan ROCK inhibition increases M1 markers CD80 and CCR7. This preserves the beneficial macrophages that are essential for retinal health 37,38, and simultaneously restores the balance between pro-angiogenic MaDAMs and their angiostatic counterparts.

Age increases ROCK signaling and the M2-type immune response, evidenced by elevated pMYPT1, pIκB-α, CCR3, and IL-4. In contrast, age does not affect the M1-type response. This establishes a novel link between age and ROCK-mediated macrophage differentiation. CD163 was only found in CNV, which could make it a candidate biomarker for proliferative AMD. Senescent MCP-1−/− mice show pronounced ROCK signaling and an M2 shift, while their M1 markers are unchanged.

The increased IκB-α phosphorylation in aged MCP-1−/− and WT mice is an intriguing finding, the relevance of which to CNV remains to be investigated. Interestingly, MCP-1−/− mice express the specific M2 marker CD163 in normal young and at higher levels in aged eyes, while normal WT eyes do not express comparable amounts of CD163. The ROCK-mediated M2-shift could thus underlie the spontaneous proliferative changes in these animals.

Demand is high for a ROCK2 specific inhibitor 17. We use an orally available selective ROCK2 inhibitor, which successfully completed the phase I clinical trial. The antagonistic role of ROCK isoforms in macrophage polarization suggests that more efficacy and fewer side effects may be possible with isoform specific inhibition. A distinct benefit of selective ROCK2 versus pan ROCK inhibition is the M1 upregulation in lesions, since selective ROCK2 inhibition does not interfere with macrophage recruitment. Instead, it restores the immunological balance that prevails in the eyes of normal young animals. Compared to anti-VEGF-A agents that treat late symptoms and have considerable deleterious effects 10, targeting macrophage differentiation would address a mechanistic root of the disease. Furthermore, since lack of ROCK isoforms does not affect VEGF-A expression, the side effects of the current anti-VEGF-A treatments would not be expected to occur with ROCK inhibition 10. This also raises the possibility of combining the anti-VEGF-A and ROCK inhibitor treatments.

AMD is commonly considered to be an inflammatory disease 39, yet the results described herein indicate an immune-modulatory feature of AMD. Elevated NF-κB signaling was found in CNV that is suppressed with ROCK inhibition. Interestingly, NF-κB can be pro- or antiangiogenic 40, yet lack of endothelial NF-κB activity does not affect CNV. This indicates a role for NF-κB signaling in other cells. Indeed, due to the key role of NF-κB in immune cells 41,42 and the up-regulated IκB-α phosphorylation in aging and CNV, it is likely that NF-κB does indeed contribute to CNV but not in the endothelium, in line with a prior report 43. The causal role of anti-inflammatory M2-like macrophages in CNV pathology challenges the conventional paradigm that AMD is predominantly inflammatory. Instead, the results described herein indicate an immune imbalance as a root of AMD.

The data presented herein permit new therapeutic approaches for immune-based therapy in age-related diseases. In conclusion, this study demonstrates the key role of ROCK in macrophage polarization and remodeling of the eye microenvironment. Aging increases ROCK2 signaling, resulting in overt expression of the pro-angiogenic MaDAMs. This newly revealed chain of events explains the angiogenic switch in the aging eye 44. In contrast, a shift of the fundus micro-environment towards M1, for instance through ROCK2 inhibition, reduces the pathology and restores the physiological macrophage balance found in the young.

### Materials & Methods

Human Tissues

CNV membranes were surgically excised from AMD and idiopathic neovascular maculopathy patients. The symptoms were documented as classic choroidal neovascularization, subfoveal or juxtafoveal choroidal neovascularization with hemorrhage and retinal detachment, when present. Average age for AMD patients (n=7) was 71.3 years, and for idiopathic neovascular maculopathy (n=7) 33.2 years. The surgeries were performed at the Surugadai Hospital of Nihon University, Japan, between 2000 and 2004. Average duration from the time of onset to surgery, 8.9 months. Average size of the choroidal neovascular lesions, 0.84 of the disc diameter. Control eyes were from healthy donors. The study followed the guidelines of theDeclaration of Helsinki. An institutional review board granted approval for allocation and histological analysis of specimens.

Animals

All animal experiments adhered to The Guiding Principles in the Care and Use of Animals (DHEW Publication, NIH 80-23) and were performed according to approved experimental protocols.

Monkeys:

Eyes from cynomolgus monkeys between 3 and 4 years of age were used in this study. Cynomolgus monkeys were restrained in a squeeze cage and injected intramuscularly in the thigh with 20 mg/kg of ketamine hydrochloride (Sankyo Yell Pharmaceutical Products Co) for general anesthesia.

Rodents:

Male C57BL/6J mice (Jackson Laboratories), weighing 24-28 g, and male Brown Norway rats (Charles River Laboratories), weighing 200 to 250 g were used in the experiments. The genetically modified mice used in this work were phenotypically normal and did not differ in weight from their WT counter parts. Animals were sheltered in ventilated plastic cages in a temperature-controlled animal facility with a 12-hour light/dark cycle and were fed standard laboratory chow and water ad libitum. In this study, young WT and MCP-1−/− were 8-12 weeks old, while aged animals were >16 months old. Male, 8-12 weeks old adiponectin−/− and IL-12p40−/− mice were purchased from Jackson labs. Macrophages from IL-12p40−/− mice have a bias toward the M2 phenotype 45, while adiponectin−/− mice have an M1 shift 16.

Tie1Cre/IκB-αΔN (Tie1ΔN) mice. The IκB-αΔN mice are knock-in mice. Briefly, the cDNA of the human NF-κB suppressor IκB-αΔN was integrated by homologous recombination in frame into the β-catenin locus 46. In the floxed IκB-αΔN mice that were used in the current work, a loxP-stop-loxP cassette was cloned in front of the transgene, resulting in the human IκB-αΔN suppressor only to be expressed in the presence of Cre 27,46,47. The expression level of IκB-αΔN in the floxed mice depends on the Cre activity and the level of β-catenin promoter activity. The latter can slightly vary in different tissues. Therefore, early in the breeding scheme, the level of IκB-αΔN expression was examined in all organs, including the peripheral blood leukocytes. IκB-αΔN was expressed in all tissues, consistent with the previously known ubiquitous distribution of β-catenin.

For this project, the IκB-αΔN mice were crossed with the Tie1Cre mice to generate the Tie1Cre/IκB-αΔN mice (or short Tie1ΔN) that were used for CNV experiments. As a result of the IκB-αΔN expression in the endothelial cells, these mice lack NF-κB signaling in their vascular endothelium.

The ROCK1+/−Tie1Cre Mice on C57Bl/6 Background.

To generate CNV lesions, pigmentation of the RPE is required, as in the C57Bl/6 background. Litters of ROCK1−/− mice with the C57Bl/6 background are markedly underrepresented, showing high lethality in utero as well as postnatally 48. A further complication is that, at birth, a large portion of the ROCK1−/− mice exhibit defects in eyelid closure (eyes-open-at-birth or EOB) and omphalocoele, which renders them unusable for eye examinations 48.

On non-pigmented backgrounds, such as FVB, the ROCK1−/− progeny does not exhibit EOB or omphalocele defects, however more than 60% of the homozygotes die in utero before E9.5 49. The majority of the pups die after birth due to cannibalism or other unknown causes, so that very few mice ever reach the experimental age 50. To have ROCK1 deficient mice on a pigmented background, tissue specific haploinsufficient ROCK1 deficient mice were generated on C57Bl/6 background. For this, Tie1-Cre ROCK1 LoxP/− (or endothelial-specific ROCK1-deficient mice) were generated by mating Tie 1-Cre recombinase knockin mice with ROCK1 LoxP/− mice 51. Heterozygote ROCK1+/−mice on C57Bl/6 background are viable and fertile with no obvious phenotypic abnormalities. They express ROCK1 in approximately half the amount of normal WT, while there is no compensatory upregulation of ROCK2 expression for the loss of ROCK1 48.

Leukocyte Transmigration Assay

To visualize the leukocyte transmigration rate, a recently introduced assay 52 was used. Mice were anesthetized with ketamine (100 mg/kg) and xylazine (10 mg/kg). Poly-HEMA pellets (0.3 μl, P3932; Sigma, St. Louis, Mo., USA) containing 400 ng MCP-1 were prepared and implanted into the corneas. Cytokine pellets were positioned at ˜0.8-1.0 mm distance from the corneal limbus. After implantation, bacitracin ophthalmic ointment (E. Fougera & Co., Melville, N.Y., USA) was applied to each eye to prevent infection.

To stain the leukocytes, 500 μl AO (1 mg/ml) was injected intravenously. Two hours after AO injection blood vessels were stained by perfusing the animals with rhodamine-labeled concanavalin A lectin (ConA; Vector Laboratories, Burlingame, Calif., USA), 10 μg/ml in PBS (pH 7.4). Briefly, under deep anesthesia, the chest cavity was opened, and a 24-gauge perfusion needle was placed into the aorta. Drainage was achieved by opening the right atrium. The animals were then perfused with 10 ml PBS to wash out blood cells in the vessels. After PBS perfusion, the animals were perfused with 5 ml rhodamine-labeled ConA and subsequently with 5 ml PBS to remove residual unbound ConA. Immediately after perfusion, the corneas were carefully removed, and flatmounts were prepared using a mounting medium (TA-030-FM, Mountant Permafluor; Lab Vision).

Cell Culture and Transfection

The monocyte cell line U937 (CRL-15932, ATCC) was maintained in RPMI-1640 supplemented with 10% FBS (Atlanta Biologicals), glutamine (2 mmol/L), penicillin (100U/mL), and streptomycin (100 μg/mL; Gibco, BRL). The mouse monocyte cell line RAW 264.7 (TIB-71, ATCC) was maintained in DMEM (30-2002, ATCC). Transfections were performed using Control—(sc-37007), ROCK1—(sc-29473h, sc-36432m) and ROCK2 (sc-29474h, sc-36433m) siRNA from Santa Cruz Biotechnology (Santa Cruz, Calif., USA) via electroporation (VCA-1004 Amaxa).

Differentiation of Bone Marrow-Derived Macrophages

Bone marrow cells were collected from femurs and tibias of wild type CL57/B6 mice. The cells were cultured in RPMI1640 medium supplemented with 20% FCS, 30% L cell sup (containing M-CSF) and P/S for 5 days. Bone marrow-derived macrophages (BMDMs) were stimulated with 1 μg/ml LPS and 20 ng/ml IFN-γ (M1 phenotype) or 20 ng/ml IL-4, IL-10 and IL-13 (M2 phenotype) for 24 hours. BMDMs were collected and washed with PBS/2 mM EDTA (cold), incubated with 5 μg/ml anti-FcγR mAb (BMφ), followed by staining with 2.5 μg/ml CD80-FITC, CD206-FITC, or isotype control. After washing with PBS/EDTA, BMDMs were analyzed using a flow cytometer (FACS Calibur).

Immunohistochemistry

Paraffin embedded sections of human eyes were deparaffinized and rehydrated with a graded alcohol series. Immunofluorescent staining was performed with antibodies (Abs) against human von Willebrandt factor (F3520, Sigma), human MMR (CD206, MAB2534, R&D Systems), human CD80 (ab53003 Abcam) or human ROCK1 (sc-17794, Santa Cruz Biotechnology) or ROCK2 (sc-1851, Santa Cruz Biotechnology) and identified with Alexa Fluor 488 (10 μg/ml, A-11055; Invitrogen) or 647 (10 μg/ml, A21244; Invitrogen) secondary Abs. On day 3 after laser injury, 10 μm frozen sections of the posterior segment were prepared. The mouse eye sections were incubated with a rat anti-mouse F4/80 mAb (MCA497R, AbD Serotec) or CD11b (550282, BD Pharmingen), and subsequently with the secondary Ab. In monkey eyes, CD68 (goat polyclonal antibody, sc-7082, Santa Cruz), vWF (rabbit polyclonal antibody, A0082, DAKO), ROCK1 (mouse monoclonal antibody, sc-17794, Santa Cruz) and ROCK2 (goat polyclonal antibody, sc-1851, Santa Cruz) were stained. Images were obtained with a Leica microscope.

Western Blot

To obtain tissues, animals were perfused with PBS and eyes were enucleated immediately after perfusion. Choroids were micro-surgically isolated and placed in 100 μl of lysis buffer (mammalian cell lysis kit MCL1, Sigma), supplemented with protease and phosphatase inhibitors (P2850, P5726, P8340 Sigma), and sonicated. The lysate was centrifuged (12000 rpm, 15 min, 4° C.) and the supernatant was collected. Each sample containing an equal amount of total protein, quantified by protein assay (Bio-Rad Laboratories), was separated by SDS-PAGE and electroblotted to PVDF membranes (Invitrogen). To block nonspecific binding, the membranes were washed with 5% skim milk and subsequently incubated with the following: rabbit Abs against phospho-MBS/MYPT1-THr853 (CY-P1025, Cyclex), MYPT1 (sc-25618, Santa Cruz Biotechnology), phospho NF-κB p65 (3033, Cell Signaling), NF-κB p65 (3034, Cell Signaling), IκB-α (9242, Cell Signaling) or mouse Abs against pIκB-α (9246, Cell Signaling), ROCK1 and ROCK2 (611136, 610623, BD Transduction Laboratories), pERM (3149, Cell Signaling), ERM (3142, Cell Signaling), IL-4 (ab11524, Abcam), CD163 (sc-33560, Santa Cruz Biotechnology), CCR3 (ab32512, Abcam), CCR7 (ab65851, Abcam), CD80 (ab53003, Abcam) and β-tubulin (ab11308, Abcam) at 4° C. overnight, followed by incubation with a horseradish peroxidase-conjugated donkey or sheep Ab against rabbit or mouse IgG (NA934V, NXA931, GE Healthcare), or goat antirat secondary (goat anti-rat IgG-HRP: sc-2032, Santa Cruz). The signals were visualized by chemiluminescence (ECL kit; GE Healthcare) according to the manufacturer's protocol.

Real Time RT-PCR

Total RNA from cultured RAW 264.7 cells was extracted using the RNeasy Plus Mini Kit (74134, Quiagen). 600 ng cDNA per sample was synthesized with TaqMan Reverse Transcription Reagents (N808-0234, Applied Biosystems) using its contained random hexamers scaled for a reaction volume of 304 Quantitative real-time PCR was performed with the TaqMan Universal PCR Master Mix (4324018, Applied Biosystems) and the respective probes: CCL22 (Hs00171080 m1, Applied Biosystems), VEGF A (Mm01281449 ml, TaqMan), and 18S rRNA (Hs99999901_s1, Applied Biosystems) as endogenous control. In BMDM experiments cDNA was synthesized from total RNA with Quantiscript Reverse Transcriptase and optimized blend of oligo-dT and random primers (Millipore).

Gene expression was measured by the change-in-threshold (AACT) method based on quantitative real-time PCR in a Light Cycler (Roche) with SYBR Green I. The primer sets for the murine Arg1 (Arginase-1; for, cctgaaggaactgaaaggaaag (SEQ ID NO: 59), rev, ttggcagatatgcagggagt (SEQ ID NO: 60)), Retnla (Fizz1; for, ccctccactgtaacgaagactc (SEQ ID NO: 61), rev, cacacccagtagcagtcatcc (SEQ ID NO: 62)), Chi313 (Ym1; for, gaacactgagctaaaaactctcctg (SEQ ID NO: 63), rev, gagaccatggcactgaacg (SEQ ID NO: 64)) and Nos2 (iNOS; for, gggctgtcacggagatca (SEQ ID NO: 65), rev, ccatgatggtcacattctgc (SEQ ID NO: 66)), β-Actin (Actb; For, catccgtaaagacctctatgccaac (SEQ ID NO: 67), Rev, accagaggcatacagggaca (SEQ ID NO: 68)) were used. Experiments were performed using Applied Biosystem's Step One Plus real-time PCR system using the company's standard cycles. The relative abundance of transcripts was normalized according to that of mouse GAPDH (4352932, Applied Biosystems), 18S rRNA.

Flow Cytometry

To examine macrophages in the retina and choroid, cells were prepared from mouse eyes. To collect a sufficient number of ocular infiltrating cells, 50 burns were delivered to mouse eyes by laser. After laser injury, eyes were enucleated at different time points (1, 2, 3, 5, and 7 days). The anterior segment (cornea, iris, and lens) was excised and the posterior segment of the eye including sclera, choroid, and retina was disrupted with scissors and then shaken in DMEM (plus 10% FBS (Gibco Laboratories), 100 U/ml penicillin, 100 μg/ml streptomycin) supplemented with 0.5 mg/ml Collagenase type D (11 088 874 103, Boehringer Mannheim) at 37° C. for 60 min. The supernatants were collected and passed through a mesh. After 3 washes, viable cells were obtained. A total of 12 eyes (6 individual pools) were examined per group. The cells were stained with PE anti-mouse CD11b (557397, BD Pharmingen), FITC antimouse CD206 (MMR, 123005; BioLegend) and PE-Cy5 anti-mouse CD80 Abs (15-0801-81, eBioscience). RAW 264.7 cells were stained with PerCP anti-mouse CD11b (101230, BioLegend), PerCP anti-mouse F4/80 (123006, BioLegend), PE anti-mouse CD80 (12-0801, eBioscience), FITC anti-mouse CD206 (141704, BioLegend).

Laser-Induced

CNV Laser-induced CNV is a frequently used acute inflammatory wound healing model that mimics the angiogenesis and leakage aspects of the disease 29. However, it does not recapitulate the complex pathogenesis of AMD, as there is no genetic component or influence of age. A non-injurious model of AMD is the senescent MCP-1−/− mouse that exhibits some features of the human disease, without the deleterious effects of acute injury 12.

To induce CNV, C57BL/6 mice were anesthetized and pupils were dilated with 5% phenylephrine and 0.8% tropicamide. Using a 532-nm laser (Oculight GLx, Iridex), a slit-lamp delivery system, and a cover glass as a contact lens, four spots (100 mW, 50 μm, 100 ms) were placed in each eye. The lesions were located at 3, 6, 9 and 12 o'clock, meridians centered on the optic nerve head and located 2 to 3 disk diameter from the optic nerve head. The same technique was used to induce CNV in Brown Norway rats (four spots, 150 mW, 100m, 100 ms) 53,54 and cynomolgus monkeys (700 mW, 100m, 100 ms) 29. Development of a bubble under laser radiation confirmed the rupture of the Bruch's membrane. Eyes showing hemorrhage were excluded from experiments.

Quantification of CNV and Leakage

Seven or fourteen days after laser injury, the size of CNV lesions was measured in choroidal flat mounts. Briefly, mice were anesthetized and perfused through the left ventricle with PBS, followed by 5 ml of 5% fluorescein isothiocyanate-dextran (FD2000S; Sigma Aldrich) in 1% gelatin. The anterior segment and retina were removed from the eyecup. The remaining retinal pigment epithelium (RPE)-choroidsclera complex was flat mounted, after relaxing radial incisions, using Mounting Medium (FM 100119, Thermo Scientific) and coverslips. Micrographs of the choroidal complex were taken with a Leica Microscope. The volume of the lesions was quantified, using confocal microscopy (Leica TCS SP2 laser scanning confocal microscope). The magnitude of the CNV lesions was determined by measuring the hyperfluorescent area with Openlab Software (Improvision). The grade of leakage was determined by Fluorescein angiography (FA).

Fluorescein Angiography

FA was performed in anesthetized Brown Norway rats from ROCK inhibitor or vehicle treated groups, using a digital fundus camera (SLO; HRA2; Heidelberg Engineering), 7 and 14 days after laser injury. Fluorescein injections were performed intravenously (0.2 ml of 2% fluorescein; Akorn, NDC 17478-253-10). Monkeys were injected with 5% fluorescein intravenously (Fluorescite, Alcon). FA images were evaluated by two masked retina specialists. The grading criteria were as follows: Grade-0, no hyperfluorescence; Grade-I, hyperfluorescence without leakage; Grade-IIA, hyperfluorescence in the early or mid-transit images and late leakage; Grade-IIB, bright hyperfluorescence in the transit images and late leakage beyond the treated areas. The Grade-IIB lesions were considered as clinically significant, as described previously 53.

### Treatments

To block both ROCK isoforms, the pan ROCK inhibitors, fasudil (20 mg/kg, H-2330; LC Laboratories, MW: 364.29) and Y-27632 (10 mg/kg, S1049, selleckchem.com) were administered daily by intraperitoneal injections. To block ROCK2, mice received twice daily intraperitoneal injections of the selective ROCK2 inhibitor (10 mg/kg, SLx-2119, SurfaceLogix, MW: 570.61). The ROCK2 selective inhibitor, SLx-2119, is currently being developed for clinical use by Kadmon® (Kadmon Corporation, LLC, NY) under the designation KD025. The control animals received equivalent amounts of vehicle, glyceryl trioctanoate (91039, Sigma-Aldrich). Intravitreal injections (5 μl, 30 μmol/l) of fasudil and ROCK2 inhibitor were performed on day 0, 3 and 6 after CNV induction, using BSS Plus as vehicle. Adult cynomolgus monkeys received intravitreous injection of fasudil (30 μM), 3 times per week. The inhibitor treatments, unless indicated otherwise, started on day 0 at the same time as CNV induction, and continued daily (fasudil once per day, ROCK2 inhibitor twice per day) until harvest.

Electroretinography (ERG)

Fasudil (30 μmol/l), ROCK2 inhibitor (30 μmol/l) and vehicle (BSS) were injected into the vitreous cavity of Brown Norway rats on days 0, 3, and 6. On day 13, rats were dark-adapted overnight and then anesthetized. Pupils were dilated using 1% tropicamide. Methods for recording dark- and light-adapted ERGs were performed as previously described 55.

Statistical Analysis

All values are expressed as mean±SEM. Data were analyzed by Student's t-test, analysis of variance (ANOVA), or chi-squared test. Differences between the experimental groups were considered statistically significant (*) or highly significant (**), when the probability value, P was <0.05 or <0.01, respectively.

### BIBLIOGRAPHY

1 Miyazaki, M. et al. The 5-year incidence and risk factors for age-related maculopathy in a general Japanese population: the Hisayama study. Invest Ophthalmol Vis Sci 46, 1907-1910, doi:46/6/1907 [pii]10.1167/iovs.04-0923 (2005).
2 Klein, R., Klein, B. E., Tomany, S. C., Meuer, S. M. & Huang, G. H. Ten-year incidence and progression of age-related maculopathy: The Beaver Dam eye study. Ophthalmology 109, 1767-1779, doi:S0161-6420(02)01146-6 [pii] (2002).
3 Klein, R. J. et al. Complement factor H polymorphism in age-related macular degeneration. Science 308, 385-389 (2005).
4 Edwards, A. O. et al. Complement factor H polymorphism and age-related macular degeneration. Science 308, 421-424 (2005).
5 Haines, J. L. et al. Complement factor H variant increases the risk of age-related macular degeneration. Science 308, 419-421 (2005).
6 Zareparsi, S. et al. Strong association of the Y402H variant in complement factor H at 1q32 with susceptibility to age-related macular degeneration. Am J Hum Genet 77, 149-153 (2005).
7 Nishijima, K. et al. Vascular endothelial growth factor-A is a survival factor for retinal neurons and a critical neuroprotectant during the adaptive response to ischemic injury. Am J Pathol 171, 53-67, doi:S0002-9440(10)61942-6 [pii] 10.2353/ajpath.2007.061237 (2007).
8 Martin, D. F. et al. Ranibizumab and bevacizumab for neovascular age-related macular degeneration. N Engl J Med 364, 1897-1908, doi:10.1056/NEJMoa1102673 (2011).
9 Maharaj, A. S. et al. VEGF and TGF-beta are required for the maintenance of the choroid plexus and ependyma. J Exp Med 205, 491-501, doi:jem.20072041 [pii] 10.1084/jem.20072041 (2008).
10 Rofagha, S., Bhisitkul, R. B., Boyer, D. S., Sadda, S. R. & Zhang, K. Seven-Year Outcomes in Ranibizumab-Treated Patients in ANCHOR, MARINA, and HORIZON: A Multicenter Cohort Study (SEVEN-UP). Ophthalmology, doi: S0161-6420(13)00331-X [pii] 10.1016/j.ophtha.2013.03.046 (2013).
11 Tsutsumi, C. et al. The critical role of ocular-infiltrating macrophages in the development of choroidal neovascularization. J Leukoc Biol 74, 25-32 (2003).
12 Ambati, J. et al. An animal model of age-related macular degeneration in senescent Ccl-2- or Ccr-2-deficient mice. Nat Med 9, 1390-1397, doi:10.1038/nm950nm950 [pii] (2003).
13 Mantovani, A. et al. The chemokine system in diverse forms of macrophage activation and polarization. Trends Immunol 25, 677-686, doi:S1471-4906(04)00295-9 [pii] 10.1016/j.it.2004.09.015 (2004).
14 Cao, X. et al. Macrophage polarization in the maculae of age-related macular degeneration: a pilot study. Pathol Int 61, 528-535, doi:10.1111/j. 1440-1827.2011.02695.x (2011).
15 Liao, X. et al. Kruppel-like factor 4 regulates macrophage polarization. J Clin Invest 121, 2736-2749, doi:45444 [pii] 10.1172/JCI45444 (2011).
16 Ohashi, K. et al. Adiponectin promotes macrophage polarization toward an anti-inflammatory phenotype. J Biol Chem 285, 6153-6160, doi:M109.088708 [pii] 10.1074/jbc.M109.088708 (2010).
17 Riento, K. & Ridley, A. J. Rocks: multifunctional kinases in cell behaviour. Nat Rev Mol Cell Biol 4, 446-456, doi:10.1038/nrm1128 nrm1128 [pii] (2003).
18 Hata, Y. et al. Antiangiogenic properties of fasudil, a potent Rho-Kinase inhibitor. Jpn J Ophthalmol 52, 16-23, doi:10.1007/s10384-007-0487-5 (2008).
19 Arita, R. et al. Rho kinase inhibition by fasudil ameliorates diabetes-induced microvascular damage. Diabetes 58, 215-226, doi:db08-0762 [pii] 10.2337/db08-0762 (2009).
20 Watanabe, K. et al. A ROCK inhibitor permits survival of dissociated human embryonic stem cells. Nat Biotechnol 25, 681-686, doi:nbt1310 [pii] 10.1038/nbt1310 (2007).
21 Pinner, S. & Sahai, E. PDK1 regulates cancer cell motility by antagonizing inhibition of ROCK1 by RhoE. Nat Cell Biol 10, 127-137, doi:ncb1675 [pii] 10.1038/ncb1675 (2008).
22 Chun, K. H. et al. Regulation of glucose transport by ROCK1 differs from that of ROCK2 and is controlled by actin polymerization. Endocrinology 153, 1649-1662, doi:en.2011-1036 [pii] 10.1210/en.2011-1036 (2012).
23 Amano, M., Nakayama, M. & Kaibuchi, K. Rho-kinase/ROCK: A key regulator of the cytoskeleton and cell polarity. Cytoskeleton (Hoboken) 67, 545-554, doi:10.1002/cm.20472 (2010).
24 Cammarano, M. S. & Minden, A. Dbl and the Rho GTPases activate NF kappa B by I kappa B kinase (IKK)-dependent and IKK-independent pathways. J Biol Chem 276, 25876-25882, doi:10.1074/jbc.M011345200 M011345200 [pii] (2001).
25 Nagumo, H. et al. Rho kinase inhibitor HA-1077 prevents Rho-mediated myosin phosphatase inhibition in smooth muscle cells. Am J Physiol Cell Physiol 278, C57-65 (2000).
26 Tabruyn, S. P. & Griffioen, A. W. NF-kappa B: a new player in angiostatic therapy. Angiogenesis 11, 101-106, doi:10.1007/s10456-008-9094-4 (2008).
27 Henke, N. et al. Vascular endothelial cell-specific NF-kappaB suppression attenuates hypertension-induced renal damage. Circ Res 101, 268-276, doi:CIRCRESAHA.107.150474 [pii] 10.1161/CIRCRESAHA.107.150474 (2007).
28 Apte, R. S., Richter, J., Herndon, J. & Ferguson, T. A. Macrophages inhibit neovascularization in a murine model of age-related macular degeneration. PLoS Med 3, e310, doi:05-PLME-RA-0725R3 [pii]10.1371/journal.pmed.0030310 (2006).
29 Ishibashi, T., Miller, H., Orr, G., Sorgente, N. & Ryan, S. J. Morphologic observations on experimental subretinal neovascularization in the monkey. Invest Ophthalmol Vis Sci 28, 1116-1130 (1987).
30 Matsumura, N. et al. Low-dose lipopolysaccharide pretreatment suppresses choroidal neovascularization via IL-10 induction. PLoS One 7, e39890, doi:10.1371/journal.pone.0039890 PONE-D-12-02235 [pii] (2012).
31 Takeda, A. et al. CCR3 is a target for age-related macular degeneration diagnosis and therapy. Nature 460, 225-230, doi:nature08151 [pii]10.1038/nature08151 (2009).
32 Kaarniranta, K. et al. Adiponectin receptor 1 gene (ADIPOR1) variant is associated with advanced age-related macular degeneration in Finnish population. Neurosci Lett 513, 233-237, doi:S0304-3940(12)00249-2 [pii] 10.1016/j.neulet.2012.02.050 (2012).
33 Leung, T., Manser, E., Tan, L. & Lim, L. A novel serine/threonine kinase binding the Ras-related RhoA GTPase which translocates the kinase to peripheral membranes. J Biol Chem 270, 29051-29054 (1995).
34 Pouliot, P., Turmel, V., Gelinas, E., Laviolette, M. & Bissonnette, E. Y. Interleukin-4 production by human alveolar macrophages. Clin Exp Allergy 35, 804-810, doi:CEA2246 [pii] 10.1111/j.1365-2222.2005.02246.x (2005).
35 Sica, A. et al. Macrophage polarization in tumour progression. Semin Cancer Biol 18, 349-355, doi:S1044-579X(08)00023-0 [pii] 10.1016/j.semcancer.2008.03.004 (2008).
36 Sun, D. et al. Superior sensitivity of novel molecular imaging probe: simultaneously targeting two types of endothelial injury markers. FASEB J 24, 1532-1540, doi:fj.09-148981 [pii] 10.1096/fj.09-148981 (2010).
37 Lang, R. A. & Bishop, J. M. Macrophages are required for cell death and tissue remodeling in the developing mouse eye. Cell 74, 453-462, doi:0092-8674(93)80047-I [pii] (1993).
38 London, A. et al. Neuroprotection and progenitor cell renewal in the injured adult murine retina requires healing monocyte-derived macrophages. J Exp Med 208, 23-39, doi:jem.20101202 [pii] 10.1084/jem.20101202 (2011).
39 Hollyfield, J. G. et al. Oxidative damage-induced inflammation initiates agerelated macular degeneration. Nat Med 14, 194-198, doi:nm1709 [pii] 10.1038/nm1709 (2008).
40 Aurora, A. B. et al. NF-kappaB balances vascular regression and angiogenesis via chromatin remodeling and NFAT displacement. Blood 116, 475-484, doi:blood-2009-07-232132 [pii] 10.1182/blood-2009-07-232132 (2010).
41 Saccani, A. et al. p50 nuclear factor-kappaB overexpression in tumor-associated macrophages inhibits M1 inflammatory responses and antitumor resistance. Cancer Res 66, 11432-11440, doi:66/23/11432 [pii] 10.1158/0008-5472.CAN-06-1867 (2006).
42 Hagemann, T. et al. “Re-educating” tumor-associated macrophages by targeting NF-kappaB. J Exp Med 205, 1261-1268, doi:jem.20080108 [pii] 10.1084/jem.20080108 (2008).
43 Jo, N. et al. Effective transfection of a cis element “decoy” of the nuclear factorkappaB binding site into the experimental choroidal neovascularization. Curr Eye Res 24, 465-473 (2002).
44 Hanahan, D. & Folkman, J. Patterns and emerging mechanisms of the angiogenic switch during tumorigenesis. Cell 86, 353-364, doi:S0092-8674(00)80108-7 [pii] (1996).
45 Bastos, K. R., Alvarez, J. M., Marinho, C. R., Rizzo, L. V. & Lima, M. R. Macrophages from IL-12p40-deficient mice have a bias toward the M2 activation profile. J Leukoc Biol 71, 271-278 (2002).
46 Schmidt-Ullrich, R. et al. Requirement of NF-kappaB/Rel for the development of hair follicles and other epidermal appendices. Development 128, 3843-3853 (2001).
47 Freund, C. et al. Requirement of nuclear factor-kappaB in angiotensin II- and isoproterenol-induced cardiac hypertrophy in vivo. Circulation 111, 2319-2325, doi:01.CIR.0000164237.58200.5A [pii] 10.1161/01.CIR.0000164237.58200.5A (2005).
48 Rikitake, Y. et al. Decreased perivascular fibrosis but not cardiac hypertrophy in ROCK1+/−haploinsufficient mice. Circulation 112, 2959-2965, doi:CIRCULATIONAHA. 105.584623 [pii] 10.1161/CIRCULATIONAHA.105.584623 (2005).
49 Shi, J., Zhang, L. & Wei, L. Rho-kinase in development and heart failure: insights from genetic models. Pediatr Cardiol 32, 297-304, doi:10.1007/s00246-011-9920-0 (2011).
50 Shimizu, Y. et al. ROCK-I regulates closure of the eyelids and ventral body wall by inducing assembly of actomyosin bundles. J Cell Biol 168, 941-953, doi:jcb. 200411179 [pii] 10.1083/jcb.200411179 (2005).
51 Huang, H. et al. Rho-kinase regulates energy balance by targeting hypothalamic leptin receptor signaling. Nat Neurosci 15, 1391-1398, doi:nn.3207 [pii] 10.1038/nn.3207 (2012).
52 Nakao, S., Zandi, S., Faez, S., Kohno, R. & Hafezi-Moghadam, A. Discontinuous LYVE-1 expression in corneal limbal lymphatics: dual function as microvalves and immunological hot spots. FASEB J 26, 808-817, doi:fj.11-183897 [pii] 10.1096411-183897 (2012).
53 Noda, K. et al. Vascular adhesion protein-1 blockade suppresses choroidal neovascularization. FASEB J 22, 2928-2935, doi:fj.07-105346 [pii] 10.1096407-105346 (2008).
54 Lara-Castillo, N. et al. Atrial natriuretic peptide reduces vascular leakage and choroidal neovascularization. Am J Pathol 175, 2343-2350, doi:ajpath.2009.090439 [pii] 10.2353/ajpath.2009.090439 (2009).
55 Pawlyk, B. S. et al. Replacement Gene Therapy with a Human RPGRIP1 Sequence Slows Photoreceptor Degeneration in a Murine Model of Leber Congenital Amaurosis. Hum Gene Ther, doi:10.1089/hum.2009.218 (2010).
56 Schueller, O., Tong, W., Ferkany, J. W. & Sweetnam, P. Abstract 1216: Selective ROCK 2 Inhibition Attenuates Arterial Plaque Formation in an ApoE Knockout Mouse Model. Circulation 114, II—228 (2007).
57 Boerma, M. et al. Comparative gene expression profiling in three primary human cell lines after treatment with a novel inhibitor of Rho kinase or atorvastatin. Blood Coagul Fibrinolysis 19, 709-718, doi:10.1097/MBC.0b013e32830b2891 00001721-200810000-00017 [pii] (2008).
58 Ishizaki, T. et al. Pharmacological properties of Y-27632, a specific inhibitor of rho-associated kinases. Mol Pharmacol 57, 976-983 (2000).
59 Lin, T. S., Neenan, J. P., Cheng, Y. C. & Prusoff, W. H. Synthesis and antiviral activity of 5- and 5′-substituted thymidine analogs. J Med Chem 19, 495-498 (1976).
60 Anastassiadis, T., Deacon, S. W., Devarajan, K., Ma, H. & Peterson, J. R. Comprehensive assay of kinase catalytic activity reveals features of kinase inhibitor selectivity. Nat Biotechnol 29, 1039-1045, doi:nbt.2017 [pii] 10.1038/nbt.2017 (2011).
61 Rikitake, Y. & Liao, J. K. ROCKs as therapeutic targets in cardiovascular diseases. Expert Rev Cardiovasc Ther 3, 441-451, doi:10.1586/14779072.3.3.441 (2005).
62 Sallusto, F., Mackay, C. R. & Lanzavecchia, A. Selective expression of the eotaxin receptor CCR3 by human T helper 2 cells. Science 277, 2005-2007 (1997).
63 Nissinen, R. et al. CCR3, CCRS, interleukin 4, and interferon-gamma expression on synovial and peripheral T cells and monocytes in patients with rheumatoid arthritis. J Rheumatol 30, 1928-1934, doi:0315162X-30-1928 [pii] (2003).
64 Huang, L., Bosch, I., Hofmann, W., Sodroski, J. & Pardee, A. B. Tat protein induces human immunodeficiency virus type 1 (HIV-1) coreceptors and promotes infection with both macrophage-tropic and T-lymphotropic HIV-1 strains. J Vivol 72, 8952-8960 (1998).

What is claimed herein is:

1. A method of treating a condition selected from the group consisting of:
pathogenic angiogenesis; vascular leakage; and aging or age-related conditions; the method comprising administering a M2 or MaDAM macrophage inhibitor to a subject.

2. The method of claim 1, wherein the M2 or MaDAM macrophage is selected from the group consisting of:
a CD11b(+) cell; a CD163(+) cell; and a CD206(+) cell.

3. The method of claim 1, wherein the pathogenic angiogenesis is associated with a condition selected from the group consisting of:
AMD, CNV, or aging.

4. The method of claim 1, wherein the vascular leakage is associated with AMD.

5. The method of claim 1, wherein the M2 macrophage inhibitor is a pan-ROCK inhibitor.

6. The method of claim 5, wherein the pan-ROCK inhibitor is selected from the group consisting of:
Fasudil; HP1152P; and Y-27632.

7. The method of claim 1, where in the M2 macrophage inhibitor is a ROCK2-specific inhibitor.

8. The method of claim 7, wherein the ROCK2 inhibitor is SLx2119.

9. The method of claim 1, wherein the M2 macrophage inhibitor is a M1-promoting cytokine.

10. The method of claim 9, wherein the M1-promoting cytokine is selected from the group consisting of:
INF-γ and LPS.

11. The method of claim 1, wherein the inhibitor is not a direct modulator of VEGF-A.

12. A method of treating a condition selected from the group consisting of:
pathogenic angiogenesis; vascular leakage; and aging or age-related conditions;
the method comprising administering a M1 macrophage to a subject.

13. The method of claim 12, wherein the M1 macrophage is administered via intravitreal injection.

14. A method of treating an inflammatory or autoimmune disease the method comprising administering a M1 macrophage inhibitor to a subject.

15. The method of claim 14, wherein the M1 macrophage inhibitor is a ROCK1 inhibitor.

16. The method of claim 15, where in the ROCK1 inhibitor is a ROCK1-specific inhibitor.

17. The method of claim 15, wherein the ROCK1 inhibitor is selected from the group consisting of:
GSK 429286; a dihydropyrimidinone; and a dihydropyrimidine.

18. The method of claim 14, wherein the M1 macrophage inhibitor is a M2-promoting cytokine.

19. The method of claim 18, wherein the M2-promoting cytokine is selected from the group consisting of:
IL-4; IL-10; and IL-13.

20. The method of claim 14, wherein the inhibitor is not a direct modulator of VEGF-A.


SEQUENCE LISTING (68 sequences)

SEQ ID NO: 1 - DNA, 6650 bp, Homo sapiens
gctggttccccttccgagcgtccgcgccccgcatgcgcagtctgccccggcggtctccgtttgtttgaacaggaaggcggacatattagtccctctcagcccccctcgccccaccccccaggcattcgccgccgcgactcgccctttccccggctgggaccgcagcccctcccagaagctcccccatcagcagccgccgggacccaactatcgtcttcctcttcgcccgctctccagcctttcctctgctaagtctccatcgggcatcgacctcgccctgccccaccggacaccgtagcagcagccccagcagcgacgggacaaaatgggagagtgaggctgtcctgcgtggaccagctcgtggccgagactgatcggtgcgtcgggccgggccgagtagagccggggacgcggggctagaccgtctacagcgcctctgagcggagcgggcccggcccgtggcccgagcggcggccgcagctggcacagctcctcacccgccctttgctttcgcctttcctcttctccctcccttgttgcccggagggagtctccaccctgcttctctttctctacccgctcctgcccatctcgggacggggacccctccatggcgacggcggccggggcccgctagactgaagcacctcgccggagcgacgaggctggtggcgacggcgctgtcggctgtcgtgaggggctgccgggtgggatgcgactttgggcgtccgagcggctgtgggtcgctgttgcccccggcccggggtctggagagcggaggtcccctcagtgaggggaagacgggggaaccgggcgcacctggtgaccctgaggttccggctcctccgccccgcggctgcgaacccaccgcggaggaagttggttgaaattgctttccgctgctggtgctggtaagagggcattgtcacagcagcagcaacatgtcgactggggacagttttgagactcgatttgaaaaaatggacaacctgctgcgggatcccaaatcggaagtgaattcggattgtttgctggatggattggatgctttggtatatgatttggattttcctgccttaagaaaaaacaaaaatattgacaactttttaagcagatataaagacacaataaataaaatcagagatttacgaatgaaagctgaagattatgaagtagtgaaggtgattggtagaggtgcatttggagaagttcaattggtaaggcataaatccaccaggaaggtatatgctatgaagcttctcagcaaatttgaaatgataaagagatctgattctgcttttttctgggaagaaagggacatcatggcttttgccaacagtccttgggttgttcagcttttttatgcattccaagatgatcgttatctctacatggtgatggaatacatgcctggtggagatcttgtaaacttaatgagcaactatgatgtgcctgaaaaatgggcacgattctatactgcagaagtagttcttgcattggatgcaatccattccatgggttttattcacagagatgtgaagcctgataacatgctgctggataaatctggacatttgaagttagcagattttggtacttgtatgaagatgaataaggaaggcatggtacgatgtgatacagcggttggaacacctgattatatttcccctgaagtattaaaatcccaaggtggtgatggttattatggaagagaatgtgactggtggtcggttggggtatttttatacgaaatgcttgtaggtgatacacctttttatgcagattctttggttggaacttacagtaaaattatgaaccataaaaattcacttacctttcctgatgataatgacatatcaaaagaagcaaaaaaccttatttgtgccttccttactgacagggaagtgaggttagggcgaaatggtgtagaagaaatcaaacgacatctcttcttcaaaaatgaccagtgggcttgggaaacgctccgagacactgtagcaccagttgtacccgatttaagtagtgacattgatactagtaattttgatgacttggaagaagataaaggagaggaagaaacattccctattcctaaagctttcgttggcaatcaactaccttttgtaggatttacatattatagcaatcgtagatacttatcttcagcaaatcctaatgataacagaactagctccaatgcagataaaagcttgcaggaaagtttgcaaaaaacaatctataagctggaagaacagctgcataatgaaatgcagttaaaagatgaaatggagcagaagtgcagaacctcaaacataaaactagacaagataatgaaagaattggatgaagagggaaatcaaagaagaaatctagaatctacagtgtctcagattgagaaggagaaaatgttgctacagcatagaattaatgagtaccaaagaaaagctgaacaggaaaatgagaagagaagaaatgtagaaaatgaagtttctacattaaaggatcagttggaagacttaaagaaagtcagtcagaattcacagcttgctaatgagaagctgtcccagttacaaaagcagctagaagaagccaatgacttacttaggacagaatcggacacagctgtaagattgaggaagagtcacacagagatgagcaagtcaattagtcagttagagtccctgaacagagagttgcaagagagaaatcgaattttagagaattctaagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatctaaccctgcaactggagcaggaatcaaataagcggctgttgttacaaaatgaattgaagactcaagcatttgaggcagacaatttaaaaggtttagaaaagcagatgaaacaggaaataaatactttattggaagcaaagagattattagaatttgagttagctcagcttacgaaacagtatagaggaaatgaaggacagatgcgggagctacaagatcagcttgaagctgagcaatatttctcgacactttataaaacccaggtaaaggaacttaaagaagaaattgaagaaaaaaacagagaaaatttaaagaaaatacaggaactacaaaatgaaaaagaaactcttgctactcagttggatctagcagaaacaaaagctgagtctgagcagttggcgcgaggccttctggaagaacagtattttgaattgacgcaagaaagcaagaaagctgcttcaagaaatagacaagagattacagataaagatcacactgttagtcggcttgaagaagcaaacagcatgctaaccaaagatattgaaatattaagaagagagaatgaagagctaacagagaaaatgaagaaggcagaggaagaatataaactggagaaggaggaggagatcagtaatcttaaggctgcctttgaaaagaatatcaacactgaacgaacccttaaaacacaggctgttaacaaattggcagaaataatgaatcgaaaagattttaaaattgatagaaagaaagctaatacacaagatttgagaaagaaagaaaaggaaaatcgaaagctgcaactggaactcaaccaagaaagagagaaattcaaccagatggtagtgaaacatcagaaggaactgaatgacatgcaagcgcaattggtagaagaatgtgcacataggaatgagcttcagatgcagttggccagcaaagagagtgatattgagcaattgcgtgctaaacttttggacctctcggattctacaagtgttgctagttttcctagtgctgatgaaactgatggtaacctcccagagtcaagaattgaaggttggctttcagtaccaaatagaggaaatatcaaacgatatggctggaagaaacagtatgttgtggtaagcagcaaaaaaattttgttctataatgacgaacaagataaggagcaatccaatccatctatggtattggacatagataaactgtttcacgttagacctgtaacccaaggagatgtgtatagagctgaaactgaagaaattcctaaaatattccagatactatatgcaaatgaaggtgaatgtagaaaagatgtagagatggaaccagtacaacaagctgaaaaaactaatttccaaaatcacaaaggccatgagtttattcctacactctaccactttcctgccaattgtgatgcctgtgccaaacctctctggcatgtttttaagccaccccctgccctagagtgtcgaagatgccatgttaagtgccacagagatcacttagataagaaagaggacttaatttgtccatgtaaagtaagttatgatgtaacatcagcaagagatatgctgctgttagcatgttctcaggatgaacaaaaaaaatgggtaactcatttagtaaagaaaatccctaagaatccaccatctggttttgttcgtgcttcccctcgaacgctttctacaagatccactgcaaatcagtctttccggaaagtggtcaaaaatacatctggaaaaactagttaaccatgtgactgagtgccctgtggaatcgtgtgggatgctacctgataaaccaggcttctttaaccatgcagagcagacaggctgtttctttgacacaaatatcacaggcttcagggttaagattgctgtttttctgtccttgctttggcacaacacactgagggttttttttattgcgggtttgcctacaggtagattagattaattattactatgtaatgcaagtacagttgggggaaagcttaggtagatatattttttttaaaaggtgctgcctttttggatttataagaaaatgcctgtcagtcgtgatagaacagagttttcctcatatgagtaagaggaagggactttcactttcaagtggaacagccatcactatcaagatcagctcatggaaggagtaaagaaaatatctcaaaatgagacaaactgaagttttgttttttttttaatgacttaagtttttgtgctcttgcaagactatacaaaactattttaagaaagcagtgatatcacttgaacttcagtgccctcactgtagaatttaaaagccttactgttgattgcccatgttggacttgatggagaaattaaatatctttcattatgctttacaaaatactgtatatgtttcagcaagtttggggaatgggagaggacaaaaaaaagttacatttaatctatgcatttttgccaagccatattgagttattttactactagagacattaggaaactaactgtacaaaagaaccaagtttaaaagcattttgtggggtacatcatttctataattgtataatgtatttctttgtggttttaaatgataaagacattaagttaacaaacatataagaaatgtatgcactgtttgaaatgtaaattattcttagaacactttcaatgggggttgcattgtccttttagtgccttaatttgagataattattttactgccatgagtaagtatagaaatttcaaaaaatgtattttcaaaaaattatgtgtgtcagtgagtttttcattgataattggtttaatttaaaatatttagaggtttgttggactttcataaattgagtacaatctttgcatcaaactacctgctacaataatgactttataaaactgcaaaaaatgtagaaggttgcaccaacataaaaaggaaatatggcaatacatccatgatgttttccagttaacataggaattaccagataaatactgttaaactcttgtccagtaacaagagttgattcatatggacagtatgatttattgtttatttttttaaccaaatacctcctcagtaatttataatggctttgcagtaatgtgtatcagataagaagcactggaaaaccgatcgtctctaggatgatatgcatgtttcaagtggtattgaaagccgcactgatggatatgtaataataaacatatctgttattaatatactaatgactctgtgctcatttaatgagaaataaaagtaatttatggatgggtatctttaatttttactgcaatgtgttttctcatggctgaaatgaatggaaaacatacttcaaattagtctctgattgtatataaatgtttgtgaaattccatggttagattaaagtgtatttttaaaagataaaa

SEQ ID NO: 2 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 3 - DNA, 6401 bp, Homo sapiens
caaggcggccggcggcgaccatggcagcgggccggcggcggccgtagtggcccaggcctgggcttcagcctcccggggccccagagggcggggcggtccgggccgcggcggtggcggcgccacttccctgctcccgcccgaggactcctgcgggcactcgctgaggaccagcggaccggcggcgcgaatctgactgaggggcggggacgccgtctgttccccgccgctcccggcagggccgggccgggctgggccgggctgggccgggcgggcccctgggagcagcccccaggcgggggaccgccttggagacccgaagccggagctagaggcaggcggtgggcccgggtggagtcccggccggagctggtggttcgggggcggtgctaggccccgaggctgcgggacctgagcgcgaggagcctgagtgcgggtccagcggtggcggcatgagccggcccccgccgacggggaaaatgcccggcgcccccgagaccgcgccgggggacggggcaggcgcgagccgccagaggaagctggaggcgctgatccgagaccctcgctcccccatcaacgtggagagcttgctggatggcttaaattccttggtccttgatttagattttcctgctttgaggaaaaacaagaacatagataatttcttaaatagatatgagaaaattgtgaaaaaaatcagaggtctacagatgaaggcagaagactatgatgttgtaaaagttattggaagaggtgcttttggtgaagtgcagttggttcgtcacaaggcatcgcagaaggtttatgctatgaagcttcttagtaagtttgaaatgataaaaagatcagattctgcctttttttgggaagaaagagatattatggcctttgccaatagcccctgggtggttcagcttttttatgcctttcaagatgataggtatctgtacatggtaatggagtacatgcctggtggagaccttgtaaaccttatgagtaattatgatgtgcctgaaaaatgggccaaattttacactgctgaagttgttcttgctctggatgcaatacactccatgggtttaatacacagagatgtgaagcctgacaacatgctcttggataaacatggacatctaaaattagcagattttggcacgtgtatgaagatggatgaaacaggcatggtacattgtgatacagcagttggaacaccggattatatatcacctgaggttctgaaatcacaagggggtgatggtttctatgggcgagaatgtgattggtggtctgtaggtgttttcctttatgagatgctagtgggggatactccattttatgcggattcacttgtaggaacatatagcaaaattatggatcataagaattcactgtgtttccctgaagatgcagaaatttccaaacatgcaaagaatctcatctgtgctttcttaacagatagggaggtacgacttgggagaaatggggtggaagaaatcagacagcatcctttctttaagaatgatcagtggcattgggataacataagagaaacggcagctcctgtagtacctgaactcagcagtgacatagacagcagcaatttcgatgacattgaagatgacaaaggagatgtagaaaccttcccaattcctaaagcttttgttggaaatcagctgcctttcatcggatttacctactatagagaaaatttattattaagtgactctccatcttgtagagaaactgattccatacaatcaaggaaaaatgaagaaagtcaagagattcagaaaaaactgtatacattagaagaacatcttagcaatgagatgcaagccaaagaggaactggaacagaagtgcaaatctgttaatactcgcctagaaaaaacagcaaaggagctagaagaggagattaccttacggaaaagtgtggaatcagcattaagacagttagaaagagaaaaggcgcttcttcagcacaaaaatgcagaatatcagaggaaagctgatcatgaagcagacaaaaaacgaaatttggaaaatgatgttaacagcttaaaagatcaacttgaagatttgaaaaaaagaaatcaaaactctcaaatatccactgagaaagtgaatcaactccagagacaactggatgaaaccaatgctttactgcgaacagagtctgatactgcagcccggttaaggaaaacccaggcagaaagttcaaaacagattcagcagctggaatctaacaatagagatctacaagataaaaactgcctgctggagactgccaagttaaaacttgaaaaggaatttatcaatcttcagtcagctctagaatctgaaaggagggatcgaacccatggatcagagataattaatgatttacaaggtagaatatgtggcctagaagaagatttaaagaacggcaaaatcttactagcgaaagtagaactggagaagagacaacttcaggagagatttactgatttggaaaaggaaaaaagcaacatggaaatagatatgacataccaactaaaagttatacagcagagcctagaacaagaagaagctgaacataaggccacaaaggcacgactagcagataaaaataagatctatgagtccatcgaagaagccaaatcagaagccatgaaagaaatggagaagaagctcttggaggaaagaactttaaaacagaaagtggagaacctattgctagaagctgagaaaagatgttctctattagactgtgacctcaaacagtcacagcagaaaataaatgagctccttaaacagaaagatgtgctaaatgaggatgttagaaacctgacattaaaaatagagcaagaaactcagaagcgctgccttacacaaaatgacctgaagatgcaaacacaacaggttaacacactaaaaatgtcagaaaagcagttaaagcaagaaaataaccatctcatggaaatgaaaatgaacttggaaaaacaaaatgctgaacttcgaaaagaacgtcaggatgcagatgggcaaatgaaagagctccaggatcagctcgaagcagaacagtatttctcaaccctttataaaacacaagttagggagcttaaagaagaatgtgaagaaaagaccaaacttggtaaagaattgcagcagaagaaacaggaattacaggatgaacgggactctttggctgcccaactggagatcaccttgaccaaagcagattctgagcaactggctcgttcaattgctgaagaacaatattctgatttggaaaaagagaagatcatgaaagagctggagatcaaagagatgatggctagacacaaacaggaacttacggaaaaagatgctacaattgcttctcttgaggaaactaataggacactaactagtgatgttgccaatcttgcaaatgagaaagaagaattaaataacaaattgaaagatgttcaagagcaactgtcaagattgaaagatgaagaaataagcgcagcagctattaaagcacagtttgagaagcagctattaacagaaagaacactcaaaactcaagctgtgaataagttggctgagatcatgaatcgaaaagaacctgtcaagcgtggtaatgacacagatgtgcggagaaaagagaaggagaatagaaagctacatatggagcttaaatctgaacgtgagaaattgacccagcagatgatcaagtatcagaaagaactgaatgaaatgcaggcacaaatagctgaagagagccagattcgaattgaactgcagatgacattggacagtaaagacagtgacattgagcagctgcggtcacaactccaagccttgcatattggtctggatagttccagtataggcagtggaccaggggatgctgaggcagatgatgggtttccagaatcaagattagaaggatggctttcattgcctgtacgaaacaacactaagaaatttggatgggttaaaaagtatgtgattgtaagcagtaagaagattcttttctatgacagtgaacaagataaagaacaatccaatccttacatggttttagatatagacaagttatttcatgtccgaccagttacacagacagatgtgtatagagcagatgctaaagaaattccaaggatattccagattctgtatgccaatgaaggagaaagtaagaaggaacaagaatttccagtggagccagttggagaaaaatctaattatatttgccacaagggacatgagtttattcctactctttatcatttcccaaccaactgtgaggcttgtatgaagcccctgtggcacatgtttaagcctcctcctgctttggagtgccgccgttgccatattaagtgtcataaagatcatatggacaaaaaggaggagattatagcaccttgcaaagtatattatgatatttcaacggcaaagaatctgttattactagcaaattctacagaagagcagcagaagtgggttagtcggttggtgaaaaagatacctaaaaagcccccagctccagacccttttgcccgatcatctcctagaacttcaatgaagatacagcaaaaccagtctattagacggccaagtcgacagcttgccccaaacaaacctagctaactgccttctatgaaagcagtcattattcaaggtgatcgtattcttccagtgaaaacaagactgaaatatgatggcccaaaatttattaaaaagctatattttcctgagagactgatacatacactcatacatatatgtgttccccttttccctgtaatataaattacaaatctgggctcctttgaagcaacaggttgaaccaacaatgattggttgatagactaaggatatatgcaactcttccagacttttccataaagctctctcggcagtcgctcacactacaatgcacacaaggattgagaagagttaaaggctaaagaaaacatcttttctagcttcaacagagaggtttcaccagcacatttaccagaagaatctgggaatggattccactacagtgatattgactgcatctttaagaagtgaccattatactgtgtatatatatataaacacacacacatatatatatatatatatagtactctaatactgcaagaaggttttttaaacttcccactttattttttatacacattaatcagatatcattacttgctgcagttgcaactatgcacttgtataaagccataatgttggagtttatatcactcattcctgtgtacctgatggaagttgcatgttcatgtttaagcagttactgtaacaagaagtttaaagttaattatatcagtttcctaatgcttcatgataggcaactttacccattttgaatgccttaatttaatttttttcaaagtctcagccctgtctgtattaaaaaacaaaaaaagcgtttaccagctcttaggatgtaaactagctttgtggaagataaatcgtgcactatttttacacataaatagttatatcaatgtcagcctattttgattaacaaatgtttttaaagtattattggttatagaaacaataatggatggtgttggaactaatatatccttgatgtctgtctattattcattcaactctttttacagacctcagtattagtctgtgactacaaaatattttatttgctttaaatttgctggctaccctagatgtgtttttattcctggtaaagacatttgtgattacattttcacacttaagattcaaaatttttcccaaatataaagaaaactaagacagactgtagatgcattttaaatatttaaatatgatcctcagacatgcagctgtgtgtggcagtattttagtaccgggttaagaaaactggcaactgggaagaagtggcctcaaaggcacttaatttgatttttattttttaaatgctgtcaaagttacagtttacgcaggacattcttgccgtattctcatgatcccagataagtgtgtgttttatactgcaacaatatgcagcaatggtaagcgtaaagttttttttttgtttttgtttttttttatattatgaagtcttttaacagtctctctttatataaatacacagagtttggtatgatatttaaatacatcatctggccaggcatggtggcttacgcctgtaatcctagcactttgggaggccaagacgggcggatcacctgaggtgaggagttcaagaccagcctgcccaacatagtgaaactccgtctctaccaatatacaaaaattagccgggcatgatggtggtggcctgtaatcccagctacttgggaggctgagacaggagaatcgcttgaacccaggagacggtggttgcagtgagcgaagatcgagccactgcactccagcctgggcagctgaacaagactccgtctc

SEQ ID NO: 4 - protein, 1388 aa, Homo sapiens
MSRPPPTGKMPGAPETAPGDGAGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFYAFQDDRYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGFYGRECDWWSVGVFLYEMLVGDTPFYADSLVGTYSKIMDHKNSLCFPEDAEISKHAKNLICAFLTDREVRLGRNGVEEIRQHPFFKNDQWHWDNIRETAAPVVPELSSDIDSSNFDDIEDDKGDVETFPIPKAFVGNQLPFIGFTYYRENLLLSDSPSCRETDSIQSRKNEESQEIQKKLYTLEEHLSNEMQAKEELEQKCKSVNTRLEKTAKELEEEITLRKSVESALRQLEREKALLQHKNAEYQRKADHEADKKRNLENDVNSLKDQLEDLKKRNQNSQISTEKVNQLQRQLDETNALLRTESDTAARLRKTQAESSKQIQQLESNNRDLQDKNCLLETAKLKLEKEFINLQSALESERRDRTHGSEIINDLQGRICGLEEDLKNGKILLAKVELEKRQLQERFTDLEKEKSNMEIDMTYQLKVIQQSLEQEEAEHKATKARLADKNKIYESIEEAKSEAMKEMEKKLLEERTLKQKVENLLLEAEKRCSLLDCDLKQSQQKINELLKQKDVLNEDVRNLTLKIEQETQKRCLTQNDLKMQTQQVNTLKMSEKQLKQENNHLMEMKMNLEKQNAELRKERQDADGQMKELQDQLEAEQYFSTLYKTQVRELKEECEEKTKLGKELQQKKQELQDERDSLAAQLEITLTKADSEQLARSIAEEQYSDLEKEKIMKELEIKEMMARHKQELTEKDATIASLEETNRTLTSDVANLANEKEELNNKLKDVQEQLSRLKDEEISAAAIKAQFEKQLLTERTLKTQAVNKLAEIMNRKEPVKRGNDTDVRRKEKENRKLHMELKSEREKLTQQMIKYQKELNEMQAQIAEESQIRIELQMTLDSKDSDIEQLRSQLQALHIGLDSSSIGSGPGDAEADDGFPESRLEGWLSLPVRNNTKKFGWVKKYVIVSSKKILFYDSEQDKEQSNPYMVLDIDKLFHVRPVTQTDVYRADAKEIPRIFQILYANEGESKKEQEFPVEPVGEKSNYICHKGHEFIPTLYHFPTNCEACMKPLWHMFKPPPALECRRCHIKCHKDHMDKKEEIIAPCKVYYDISTAKNLLLLANSTEEQQKWVSRLVKKIPKKPPAPDPFARSSPRTSMKIQQNQSIRRPSRQLAPNKPS

SEQ ID NO: 5 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 6 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 7 - DNA, 4745 bp, Homo sapiens
ttttctgcccttctttgctttggtggcttccttgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggcagagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacgctccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcaagtgtctgtgtgcaagtgtgtgcacatgtgtgcgtgtgcgtgcatgtgcacttgcacgcccatgtgtgagtgtgtgcaagtatgtgagtgtgtccaagtgtgtgtgcgtgtgtccatgtgtgtgcaagtgtgtgcatgtgtgcgagtgtgtgcatgtgtgtgctcaggggcgtgtggctcacgtgtgtgactcagatgtctctggcgtgtgggtaggtgacggcagcgtagcctctccggcagaagggaactgcctgggctcccttgtgcgtgggtgaagccgctgctgggttttcctccgggagaggggacggtcaatcctgtgggtgaagacagagggaaacacagcagcttctctccactgaaagaagtgggacttcccgtcgcctgcgagcctgcggcctgctggagcctgcgcagcttggatggagactccatgagaagccgtgggtggaaccaggaacctcctccacaccagcgctgatgcccaataaagatgcccactgaggaatgatgaagcttcctttctggattcatttattatttcaatgtgactttaattttttggatggataagcttgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacacaccacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcattctcttgttattgcatcaatgctgagttaataaatcaaatatatgtcatttttgcatatatgtaaggataa

SEQ ID NO: 8 - protein, 1153 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 9 - DNA, 4190 bp, Homo sapiens
atatgtagccttttcattttcatgaaagtgaagtgatttttagaattcttagttgttttctttagaagaacatttctagggaataatacaagaagatttaggaatcattgaagttataaatctttggaatgagcaaactcagaatggtgctacttgaagactctggatctgctgacttcagaagacattttgtcaacttgagtcccttcaccattactgtggtcttacttctcagtgcctgttttgtcaccagttctcttggaggaacagacaaggagctgaggctagtggatggtgaaaacaagtgtagcgggagagtggaagtgaaagtccaggaggagtggggaacggtgtgtaataatggctggagcatggaagcggtctctgtgatttgtaaccagctgggatgtccaactgctatcaaagcccctggatgggctaattccagtgcaggttctggacgcatttggatggatcatgtttcttgtcgtgggaatgagtcagctctttgggattgcaaacatgatggatggggaaagcatagtaactgtactcaccaacaagatgctggagtgacctgctcagatggatccaatttggaaatgaggctgacgcgtggagggaatatgtgttctggaagaatagagatcaaattccaaggacggtggggaacagtgtgtgatgataacttcaacatagatcatgcatctgtcatttgtagacaacttgaatgtggaagtgctgtcagtttctctggttcatctaattttggagaaggctctggaccaatctggtttgatgatcttatatgcaacggaaatgagtcagctctctggaactgcaaacatcaaggatggggaaagcataactgtgatcatgctgaggatgctggagtgatttgctcaaagggagcagatctgagcctgagactggtagatggagtcactgaatgttcaggaagattagaagtgagattccaaggagaatgggggacaatatgtgatgacggctgggacagttacgatgctgctgtggcatgcaagcaactgggatgtccaactgccgtcacagccattggtcgagttaacgccagtaagggatttggacacatctggcttgacagcgtttcttgccagggacatgaacctgctatctggcaatgtaaacaccatgaatggggaaagcattattgcaatcacaatgaagatgctggcgtgacatgttctgatggatcagatctggagctaagacttagaggtggaggcagccgctgtgctgggacagttgaggtggagattcagagactgttagggaaggtgtgtgacagaggctggggactgaaagaagctgatgtggtttgcaggcagctgggatgtggatctgcactcaaaacatcttatcaagtgtactccaaaatccaggcaacaaacacatggctgtttctaagtagctgtaacggaaatgaaacttctctttgggactgcaagaactggcaatggggtggacttacctgtgatcactatgaagaagccaaaattacctgctcagcccacagggaacccagactggttggaggggacattccctgttctggacgtgttgaagtgaagcatggtgacacgtggggctccatctgtgattcggacttctctctggaagctgccagcgttctatgcagggaattacagtgtggcacagttgtctctatcctggggggagctcactttggagagggaaatggacagatctgggctgaagaattccagtgtgagggacatgagtcccatctttcactctgcccagtagcaccccgcccagaaggaacttgtagccacagcagggatgttggagtagtctgctcaagatacacagaaattcgcttggtgaatggcaagaccccgtgtgagggcagagtggagctcaaaacgcttggtgcctggggatccctctgtaactctcactgggacatagaagatgcccatgttctttgccagcagcttaaatgtggagttgccctttctaccccaggaggagcacgttttggaaaaggaaatggtcagatctggaggcatatgtttcactgcactgggactgagcagcacatgggagattgtcctgtaactgctctaggtgcttcattatgtccttcagagcaagtggcctctgtaatctgctcaggaaaccagtcccaaacactgtcctcgtgcaattcatcgtctttgggcccaacaaggcctaccattccagaagaaagtgctgtggcctgcatagagagtggtcaacttcgcctggtaaatggaggaggtcgctgtgctgggagagtagagatctatcatgagggctcctggggcaccatctgtgatgacagctgggacctgagtgatgcccacgtggtttgcagacagctgggctgtggagaggccattaatgccactggttctgctcattttggggaaggaacagggcccatctggctggatgagatgaaatgcaatggaaaagaatcccgcatttggcagtgccattcacacggctgggggcagcaaaattgcaggcacaaggaggatgcgggagttatctgctcagaattcatgtctctgagactgaccagtgaagccagcagagaggcctgtgcagggcgtctggaagttttttacaatggagcttggggcactgttggcaagagtagcatgtctgaaaccactgtgggtgtggtgtgcaggcagctgggctgtgcagacaaagggaaaatcaaccctgcatctttagacaaggccatgtccattcccatgtgggtggacaatgttcagtgtccaaaaggacctgacacgctgtggcagtgcccatcatctccatgggagaagagactggccagcccctcggaggagacctggatcacatgtgacaacaagataagacttcaggaaggacccacttcctgttctggacgtgtggagatctggcatggaggttcctgggggacagtgtgtgatgactcttgggacttggacgatgctcaggtggtgtgtcaacaacttggctgtggtccagctttgaaagcattcaaagaagcagagtttggtcaggggactggaccgatatggctcaatgaagtgaagtgcaaagggaatgagtcttccttgtgggattgtcctgccagacgctggggccatagtgagtgtgggcacaaggaagacgctgcagtgaattgcacagatatttcagtgcagaaaaccccacaaaaagccacaacaggtcgctcatcccgtcagtcatcctttattgcagtcgggatccttggggttgttctgttggccattttcgtcgcattattcttcttgactaaaaagcgaagacagagacagcggcttgcagtttcctcaagaggagagaacttagtccaccaaattcaataccgggagatgaattcttgcctgaatgcagatgatctggacctaatgaattcctcagaaaattcccatgagtcagctgatttcagtgctgctgaactaatttctgtgtctaaatttcttcctatttctggaatggaaaaggaggccattctgagccacactgaaaaggaaaatgggaatttataacccagtgagttcagcctttaagataccttgatgaagacctggactattgaatggagcagaaattcacctctctcactgactattacagttgcatttttatggagttcttcttctcctaggattcctaagactgctgctgaatttataaaaattaagtttgtgaatgtgactacttagtggtgtatatgagactttcaagggaattaaataaataaataagaatgttattgatttgagtttgctttaattacttgtccttaattctattaatttctaaatgggcttcctaattttttgtagagtttcctagatgtattataatgtgttttatttgacagtgtttcaatttgcatatacagtactgtatattttttcttatttggtttgaataattttcctattaccaaataaaaataaatttatttttactttagtttttctaagacaggaaaagttaatgatattgaagggtctgtaaataatatatggctaactttataaggcatgactcacaacgattctttaactgctttttgttactgtaattctgttcactagaataaaatgcagagccacacctggtgagggcac

SEQ ID NO: 10 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 11 - DNA, 5205 bp, Homo sapiens
cttttcagctgggcagctctgggaacttggattaggtggagaggcagttggggggcctcgttgttttgcgtcttagttccgccctcctgtccatcaggagaaggaaaggataaaccctgggccatgaggctacccctgctcctggtttttgcctctgtcattccgggtgctgttctcctactggacaccaggcaatttttaatctataatgaagatcacaagcgctgcgtggatgcagtgagtcccagtgccgtccaaaccgcagcttgcaaccaggatgccgaatcacagaaattccgatgggtgtccgaatctcagattatgagtgttgcatttaaattatgcctgggagtgccatcaaaaacggactgggttgctatcactctctatgcctgtgactcaaaaagtgaatttcagaaatgggagtgcaaaaatgacacacttttggggatcaaaggagaagatttattttttaactacggcaacagacaagaaaagaatattatgctctacaagggatcgggtttatggagcaggtggaagatctatggaaccacagacaatctgtgctccagaggttatgaagccatgtatacgctactaggcaatgccaatggagcaacctgtgcattcccgttcaagtttgaaaacaagtggtacgcagattgcacgagtgctgggcggtcggatggatggctctggtgcggaaccactactgactatgacacagacaagctatttggatattgtccattgaaatttgagggcagtgaaagcttatggaataaagacccgctgaccagcgtttcctaccagataaactccaaatccgctttaacgtggcaccaggcgaggaaaagctgccaacaacagaacgctgagctcctgagcatcacagagattcatgagcaaacatacctgacaggattaaccagttccttgacctcaggactctggattggacttaacagtctgagcttcaacagcggttggcagtggagtgaccgcagtcctttccgatatttgaactggttaccaggaagtccatcagctgaacctggaaaaagctgtgtgtcactaaatcctggaaaaaatgctaaatgggaaaatctggaatgtgttcagaaactgggctatatttgcaaaaagggcaacaccactttaaattcttttgttattccctcagaaagtgatgtgcctactcactgtcctagtcagtggtggccgtatgccggtcactgttacaagattcacagagatgagaaaaaaatccagagggatgctctgaccacctgcaggaaggaaggcggtgacctcacaagtatccacaccatcgaggaattggactttattatctcccagctaggatatgagccaaatgacgaattgtggatcggcttaaatgacattaagattcaaatgtactttgagtggagtgatgggacccctgtaacgtttaccaaatggcttcgtggagaaccaagccatgaaaacaacagacaggaggattgtgtggtgatgaaaggcaaggatgggtactgggcagatcggggctgtgagtggcctcttggctacatctgcaagatgaaatcacgaagccaaggtccagaaatagtggaagtcgaaaaaggctgcaggaaaggctggaaaaaacatcacttttactgctatatgattggacatacgctttcaacatttgcagaagcaaaccaaacctgtaataatgagaatgcttatttaacaactattgaagacagatatgaacaagccttcctgactagtttcgttggcttaaggcctgaaaaatatttctggacaggactttcagatatacaaaccaaagggacttttcagtggaccatcgaggaagaggttcggttcacccactggaattcagatatgccagggcgaaagccagggtgtgttgccatgagaaccgggattgcagggggcttatgggatgttttgaaatgtgatgaaaaggcaaaatttgtgtgcaagcactgggcagaaggagtaacccacccaccgaagcccacgacgactcccgaacccaaatgtccggaggattggggcgccagcagtagaacaagcttgtgtttcaagctgtatgcaaaaggaaaacatgagaagaaaacgtggtttgaatctcgagatttttgtcgagctctgggtggagacttagctagcatcaataacaaagaggaacagcaaacaatatggcgattaataacagctagtggaagctaccacaaactgttttggttgggattgacatatggaagcccttcagaaggttttacttggagtgatggttctcctgtttcatatgaaaactgggcttatggagaacctaataattatcaaaatgttgaatactgtggtgagctgaaaggtgaccctactatgtcttggaatgatattaattgtgaacaccttaacaactggatttgccagatacaaaaaggacaaacaccaaaacctgagccaacaccagctcctcaagacaatccaccagttactgaagatgggtgggttatttacaaagactaccagtattatttcagcaaagagaaggaaaccatggacaatgcgcgagcgttttgcaagaggaattttggtgatcttgtttctattcaaagtgaaagtgaaaagaagtttctatggaaatatgtaaacagaaatgatgcacagtctgcatattttattggtttattgatcagcttggataaaaagtttgcttggatggatggaagcaaagtggattacgtgtcttgggccacaggtgaacccaattttgcaaatgaagatgaaaactgtgtgaccatgtattcaaattcagggttttggaatgacattaactgtggctatccaaacgccttcatttgccagcgacataacagtagtatcaatgctaccacagttatgcctaccatgccctcggtcccatcagggtgcaaggaaggttggaatttctacagcaacaagtgtttcaaaatctttggatttatggaagaagaaagaaaaaattggcaagaggcacgaaaagcttgtataggctttggagggaatctggtctccatacaaaatgaaaaagagcaagcatttcttacctatcacatgaaggactccactttcagtgcctggactgggctgaatgatgtcaattcagaacacacgttcctttggacggatggacgaggagtccattacacaaactgggggaaaggttaccctggtggaagaagaagcagtctttcttatgaagatgctgactgtgttgttattattggaggtgcatcaaatgaagcaggaaaatggatggatgatacctgcgacagtaaacgaggctacatatgccagacacgatccgacccttccttgactaatcctccagcaacgattcaaacagatggctttgttaaatatggcaaaagcagctattcactcatgagacaaaaatttcaatggcatgaagcggagacatactgcaagcttcacaattcccttatagccagcattctggatccctacagtaatgcatttgcgtggctgcagatggaaacatctaatgaacgtgtgtggatcgccctgaacagtaacttgactgataatcaatacacttggactgataagtggagggtgaggtacactaactgggctgctgatgagcccaaattgaaatcagcatgtgtttatctggatcttgatggctactggaagacagcacattgcaatgaaagtttttactttctctgtaaaagatcagatgaaatccctgctactgaacccccacaactgcctggcagatgcccggagtcagatcacacagcatggattcctttccatggtcactgttactatattgagtcctcatatacaagaaactggggccaagcttctctggaatgtcttcgaatgggttcctctctggtttccattgaaagtgctgcagaatccagttttctgtcatatcgggttgagccacttaaaagtaaaaccaatttttggataggattgttcagaaatgttgaagggacgtggctgtggataaataacagtccggtctcctttgtcaactggaacacaggagatccctctggtgaacggaatgattgtgtagctttacatgcgtcttctgggttttggagtaatattcactgttcatcctacaaaggatatatttgtaaaagaccaaaaattattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttaacagtcagtcaagcccaggaactagtgatatgaaagatctcgtgggcaatattgaacagaatgaacactcggtcatctagtacctcaatgcgattctgagatatttgaatttcataaaattgtaactgaaatttaaaatttttagttcaatgtgattgttttctttaaaatgagtactgaattgtactggtctgtccttttttcctttgcctaattgaagaaataattgcttgttttctagcctggcaagatattttcataaaagagggataacaatgctgattactaccttttaaaatattttagataaatgcacagcaccacagcaccacatctaagcattagtgatgggtagctgatgtcagcttcatgtggattttaagcactctagaaacaatgaagcttcttggcatattttaaggagctcccaaaatgtgttacctattaaattgtaactcagcaagtagaagaccatttgaaaagtcaggtacaaatttcctcaagtggcataaaaatgtagtcagttttctcttttaccagtttttatttccactccaattatttagaactttatttgtacatgtgcagaagaataaggcagctgagaatcttgtttcccccaagagagttttacaggctgagtgttgcaaatgtgttctttgtcctgttatatgtatatcaggaatacaaggatgtgaaataaaactgtaaatttgcataactggatgtacttagataatgtgaaataaacattaaagacaaggtctatttttaataaaaaaaaaaaaaaa

SEQ ID NO: 12 - protein, 1456 aa, Homo sapiens [sequence omitted]

SEQ ID NO: 13 - DNA, 642 bp, Homo sapiens
tgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaaaaaaaaaaaaaaaaaaaaaaaaaa

SEQ ID NO: 14 - protein, 153 aa, Homo sapiens [sequence omitted]

SEQ ID NO: 15 - DNA, 1629 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 16 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - DNA, 1282 bp, Homo sapiens
aagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagat

SEQ ID NO: 18 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 19 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 20 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 21 - DNA, 1049 bp, Homo sapiens
aaaacaacaggaagcagcttacaaactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaa

SEQ ID NO: 22 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 23 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 24 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 25 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 26 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 27 - DNA, 813 bp, Homo sapiens
agctggtttcagacttcagaaggacacgggcagcagacagtggtcagtcctttcttggctctgctgacactcgagcccacattccgtcacctgctcagaatcatgcaggtctccactgctgcccttgctgtcctcctctgcaccatggctctctgcaaccagttctctgcatcacttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaaaaaaaaaaaaaaaaaa

SEQ ID NO: 28 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 29 - DNA, 1319 bp, Homo sapiens
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 30 - protein, 154 aa, Homo sapiens
MKVSAAALAVILIATALCAPASASPYSSDTTPCCFAYIARPLPRAHIKEYFYTSGKCSNPAVVHRSRMPKREGQQVWQDFLYDSRLNKGKLCHPKEPPSVCQPREEMGSGVHQLFGDELGWRVLEPELTQICLFLLALVLAWEASPHYPTPPAP

SEQ ID NO: 31 - DNA, 2757 bp, Homo sapiens
gacaagtactgagtgaactcaaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtcccacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggtgttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataa

SEQ ID NO: 32 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 33 - DNA, 2207 bp, Homo sapiens
cacttcctccccagacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 34 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 35 - DNA, 4206 bp, Homo sapiens
ataactttgtagcgagtcgaaaactgaggctccggccgcagagaactcagcctcattcctgctttaaaatctctcggccacctttgatgaggggactgggcagttctagacagtcccgaagttctcaaggcacaggtctcttcctggtttgactgtccttaccccggggaggcagtgcagccagctgcaagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccagtccagtgacacaggatgaccttcagtatcacaacctcagcaagcagcagaatgagtccccgcagcccctcgtggagacgggaaagaagtctccagaatctctggtcaagctggatgcaaccccattgtcctccccacggcatgtgaggatcaaaaactggggcagcgggatgactttccaagacacacttcaccataaggccaaagggattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagaggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtcttcgatgcccgcagctgttccactgcccgggaaatgtttgaacacatctgcagacacgtgcgttactccaccaacaatggcaacatcaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaaatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggaagtgggcaggagaatgggcctggaaacgcacaagctggcctcgctctggaaagaccaggctgtcgttgagatcaacattgctgtgctccatagtttccagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggtagaggcctggaaaacccatgtctggcaggacgagaagcggagacccaagagaagagagattccattgaaagtcttggtcaaagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggttgtctgcatggataagtacaggctgagctgcctggaggaggaacggctgctgttggtggtgaccagtacgtttggcaatggagactgccctggcaatggagagaaactgaagaaatcgctcttcatgctgaaagagctcaacaacaaattcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggcagcctgtgagacgtttgatgtccgaggcaaacagcacattcagatccccaagctctacacctccaatgtgacctgggacccgcaccactacaggctcgtgcaggactcacagcctttggacctcagcaaagccctcagcagcatgcatgccaagaacgtgttcaccatgaggctcaaatctcggcagaatctacaaagtccgacatccagccgtgccaccatcctggtggaactctcctgtgaggatggccaaggcctgaactacctgccgggggagcaccttggggtttgcccaggcaaccagccggccctggtccaaggtatcctggagcgagtggtggatggccccacaccccaccagacagtgcgcctggaggccctggatgagagtggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacagagatccacctgactgtggccgtggtcacctaccacacccgagatggccagggtcccctgcaccacggcgtctgcagcacatggctcaacagcctgaagccccaagacccagtgccctgctttgtgcggaatgccagcggcttccacctccccgaggatccctcccatccttgcatcctcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagcccagcagcctggagatgtcagcgctctgagggcctacaggaggggttaaagctgccggcacagaacttaaggatggagccagctctgcattatctgaggtcacagggcctggggagatggaggaaagtgatatcccccagcctcaagtcttatttcctcaacgttgctccccatcaagccctttacttgacctcctaacaagtagcaccctggattgatcggagcctcctctctcaaactggggcctccctggtcccttggagacaaaatcttaaatgccaggcctggcaagtgggtgaaagatggaacttgctgctgagtgcaccacttcaagtgaccaccaggaggtgctatcgcaccactgtgtatttaactgccttgtgtacagttatttatgcctctgtatttaaaaaactaacacccagtctgttccccatggccacttgggtcttccctgtatgattccttgatggagatatttacatgaattgcattttactttaatcaca

SEQ ID NO: 36 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 37 - DNA, 2583 bp, Homo sapiens
agccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggcc

SEQ ID NO: 38 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 39 - DNA, 2933 bp, Homo sapiens
gcagacacctgggctgagacatacaggacagagcatggatcgcctacagactgcactcctggttgtcctcgtcctccttgctgtggcgcttcaagcaactgaggcaggcccctacggcgccaacatggaagacagcgtctgctgccgtgattacgtccgttaccgtctgcccctgcgcgtggtgaaacacttctactggacctcagactcctgcccgaggcctggcgtggtgttgctaaccttcagggataaggagatctgtgccgatcccagagtgccctgggtgaagatgattctcaataagctgagccaatgaagagcctactctgatgaccgtggccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccctgacccctctaacccatcctctgcctccctccctgcagtcagagggtcctgttcccatcagcgattcccctgcttaaacccttccatgactccccactgccctaagctgaggtcagtctcccaagcctggcatgtggccctctggatctgggttccatctctgtctccagcctgcccacttcccttcatgaatgttgggttctagctccctgttctccaaacccatactacacatcccacttctgggtctttgcctgggatgttgctgacacccagaaagtcccaccacctgcacatgtgtagccccaccagccctccaaggcattgctcgcccaagcagctggtaattccatttcatgtattagatgtcccctggccctctgtcccctcttaataaccctagtcacagtctccgcagattcttgggatttgggggttttctcccccacctctccactagttggaccaaggtttctagctaagttactctagtctccaagcctctagcatagagcactgcagacaggccctggctcagaatcagagcccagaaagtggctgcagacaaaatcaataaaactaatgtccctcccctctccctgccaaaaggcagttacatatcaatacagagactcaaggtcactagaaatgggccagctgggtcaatgtgaagccccaaatttgcccagattcacctttcttcccccactccctttttttttttttttttgagatggagtttcgctcttgtcacccacgctggagtgcaatggtgtggtcttggcttattgaagcctctgcctcctgggttcaagtgattctcttgcctcagcctcctgagtagctgggattacaggttcctgctaccacgcccagctaatttttgtatttttagtagagacgaggcttcaccatgttggccaggctggtctcgaactcctgtcctcaggtaatccgcccacctcagcctcccaaagtgctgggattacaggcgtgagccacagtgcctggcctcttccctctccccaccccccccccaacttttttttttttttatggcagggtctcactctgtcgcccaggctggagtgcagtggcgtgatctcggctcactacaacctcgacctcctgggttcaagcgattctcccaccccagcctcccaagtagctgggattacaggtgtgtgccactacggctggctaatttttgtatttttagtagagacaggtttcaccatattggccaggctggtcttgaactcctgacctcaagtgatccaccttccttgtgctcccaaagtgctgagattacaggcgtgagctatcacacccagcctccccctttttttcctaataggagactcctgtacctttcttcgttttacctatgtgtcgtgtctgcttacatttccttctcccctcaggctttttttgggtggtcctccaacctccaatacccaggcctggcctcttcagagtaccccccattccactttccctgcctccttccttaaatagctgacaatcaaattcatgctatggtgtgaaagactacctttgacttggtattataagctggagttatatatgtatttgaaaacagagtaaatacttaagaggccaaatagatgaatggaagaattttaggaactgtgagagggggacaaggtggagctttcctggccctgggaggaagctggctgtggtagcgtagcgctctctctctctgtctgtggcaggaggcaaagagtagggtgtaattgagtgaaggaatcctgggtagagaccattctcaggtggttgggccaggctaaagactgggatttgggtctatctatgcctttctggctgatttttgtagagacggggttttgccatgttacccaggctggtctcaaactcctgggctcaagcgatcctcctggctcagcctcccaaagtgctgggattacaggcgtgagtcactgcgcctggcttcctcttcctcttgagaaatattcttttcatacagcaagtatgggacagcagtgtcccaggtaaaggacataaatgttacaagtgtctggtcctttctgagggaggctggtgccgctctgcagggtatttgaacctgtggaattggaggaggccatttcactccctgaacccagcctgacaaatcacagtgagaatgttcaccttataggcttgctgtggggctcaggttgaaagtgtggggagtgacactgcctaggcatccagctcagtgtcatccagggcctgtgtccctcccgaacccagggtcaacctgcctaccacaggcactagaaggacgaatctgcctactgcccatgaacggggccctcaagcgtcctgggatctccttctccctcctgtcctgtccttgcccctcaggactgctggaaaataaatcctttaaaatagtaaaaaaaaaaaaaaa

SEQ ID NO: 40 - protein, 93 aa, Homo sapiens
MDRLQTALLVVLVLLAVALQATEAGPYGANMEDSVCCRDYVRYRLPLRVVKHFYWTSDSCPRPGVVLLTFRDKEICADPRVPWVKMILNKLSQ

SEQ ID NO: 41 - DNA, 1567 bp, Mus musculus
aaaaagccagcagaagctctccagaagcaatcctgaagacaccatggccaagctcattcttgtcacaggtctggcaattcttctgaacgtacagctgggatcttcctaccagctgatgtgctactataccagttgggctaaggacaggccaatagaagggagtttcaaacctggtaatattgacccctgcctgtgtactcacctgatctatgcctttgctggaatgcagaataatgagatcacttacacacatgagcaagacttgcgtgactatgaagcattgaatggtctgaaagacaagaacactgagctaaaaactctcctggccattggaggatggaagtttggacctgccccgttcagtgccatggtctctactcctcagaaccgtcagatattcattcagtcagttatcagattccttcgtcaatataactttgatggcctcaacctggactggcagtaccctgggtctcgaggaagccctcctaaggacaaacatctcttcagtgttctggtgaaggaaatgcgtaaagcttttgaggaagaatctgtggagaaagacattccaaggctgctactcacttccacaggagcaggaatcattgacgtaatcaagtctgggtacaagatccctgaactgtctcagtctcttgactatattcaggtcatgacatatgatctccatgatcctaaggatggctacactggagaaaatagtcccctctataaatctccatatgacattggaaagagtgctgatctcaatgtggattcaatcatttcctactggaaggaccatggagcagcttctgagaagctcattgtgggatttccagcatatgggcatacctttatcctgagtgacccttctaagactggaattggtgcccctacaattagtactggcccaccaggaaagtacacagatgaatcaggactcctggcttactatgaggtttgtacatttctgaatgaaggagccactgaggtctgggatgccccccaggaagtaccctatgcctatcagggtaatgagtgggttggttatgacaatgtcaggagcttcaagttgaaggctcagtggctcaaggacaacaatttaggaggtgccgtggtctggcccctggacatggatgacttcagtggttctttctgtcaccagagacatttccctctgacatctactttaaagggagatctcaatatacacagtgcaagttgcaagggcccttattgagaggagctttacacaatgatttgtccttgaaactctcagaataagatcaagttcaacggtttttccacagtgcattctgcatcatgcttccatggagaataatagaaataagtcatgaactttcctaaattgaatcccagagtagtactaagatggatgtcttgtctgctgtaccagctgggaagaaacaaaaaatgctcttcatctgtcagctttggctaagctctgaacatcttttgcttcctgtaaaaccaccatgcttgtttcttgctctcacaataaattccacattcatagcaaaaaaaaaaaaaaaaaaaaaaaaaaaaa

SEQ ID NO: 42 - protein, 398 aa, Mus musculus
MAKLILVTGLAILLNVQLGSSYQLMCYYTSWAKDRPIEGSFKPGNIDPCLCTHLIYAFAGMQNNEITYTHEQDLRDYEALNGLKDKNTELKTLLAIGGWKFGPAPFSAMVSTPQNRQIFIQSVIRFLRQYNFDGLNLDWQYPGSRGSPPKDKHLFSVLVKEMRKAFEEESVEKDIPRLLLTSTGAGIIDVIKSGYKIPELSQSLDYIQVMTYDLHDPKDGYTGENSPLYKSPYDIGKSADLNVDSIISYWKDHGAASEKLIVGFPAYGHTFILSDPSKTGIGAPTISTGPPGKYTDESGLLAYYEVCTFLNEGATEVWDAPQEVPYAYQGNEWVGYDNVRSFKLKAQWLKDNNLGGAVVWPLDMDDFSGSFCHQRHFPLTSTLKGDLNIHSASCKGPY

SEQ ID NO: 43 - DNA, 594 bp, Homo sapiens
ccacgttgtcttctttccttcaccaccacccaggagctcagagatctaagctgctttccatcttttctcccagccccaggacactgactctgtacaggatggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccacttcatttgttccattcctgattcttgggtaataaagacaaactttgtacctcaaaaaaaaaaaaaaaaaaaaa

SEQ ID NO: 44 - protein, 111 aa, Homo sapiens
MGPSSCLLLILIPLLQLINPGSTQCSLDSVMDKKIKDVLNSLEYSPSPISKKLSCASVKSQGRPSSCPAGMAVTGCACGYGCGSWDVQLETTCHCQCSVVDWTTARCCHLT

SEQ ID NO: 45 - DNA, 1499 bp, Homo sapiens
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 46 - protein, 330 aa, Homo sapiens
MSAKSRTIGIIGAPFSKGQPRGGVEEGPTVLRKAGLLEKLKEQVTQNFLILECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 47 - DNA, 5582 bp, Homo sapiens
agatctgccctgtagagccttgcggttccactgctggcctccggattcccgggagcccccagcccgacaggacaaccttccttccccgcttcctcctttccagacccggcggcaggagaggggatgaagatggcggacgcgaagcagaagcggaacgagcagctgaaacgctggatcggctccgagacggacctcgagcctccggtggtgaagcgccagaagaccaaggtgaagttcgacgatggcgccgtcttcctggctgcttgctccagcggcgacacggacgaggtcctcaagctgctgcaccgcggcgccgacatcaattacgccaatgtggacggactcactgccctgcaccaggcttgcattgatgacaatgttgatatggtgaagtttctggtagaaaatggagcaaatattaatcaacctgataatgaaggctggataccactacatgcagcagcttcctgtggatatcttgatattgcagagtttttgattggtcaaggagcacatgtaggggctgtcaacagtgaaggagatacacctttagatattgcggaggaggaggcaatggaagagctacttcaaaatgaagttaatcggcaaggggttgatatagaagcagctcgaaaggaagaagaacggatcatgcttagagatgccaggcagtggctaaatagtggtcatataaatgatgtccggcatgcaaaatctggaggtacagcacttcacgttgcagctgctaaaggctatacggaagttttaaaacttttaatacaggcaggctatgatgttaatattaaagactatgatggctggacacctcttcatgctgcagctcattggggtaaagaagaagcatgtcgaattttagtggacaatctgtgtgatatggagatggtcaacaaagtgggccaaacagcctttgatgtagcagatgaagacattttaggatatttagaagagttgcaaaagaaacaaaatctgctccatagtgaaaaacgggacaagaaatctccactaattgaatcaacagcaaatatggacaataatcagtcacagaagacctttaaaaacaaagagacgttgattattgaaccagagaaaaatgcatcccgtattgaatctctggaacaagaaaaggttgatgaagaagaagaaggaaagaaggatgagtctagctgctctagtgaagaagatgaggaagatgactcggaatcagaagctgaaacagataagacaaaacccctggcttctgtaactaatgccaacacttctagtacacaagcagctcctgtagctgttacaacacctactgtgtcatcaggtcaagcaacacctacatcacctattaaaaagtttccaaccacagctacaaaaatttctcccaaagaagaagagagaaaagatgagtctcctgcaacttggaggttaggacttagaaagacgggcagctatggtgcacttgctgaaatcacagcatctaaagagggtcagaaagaaaaagatactgcaggtgttacacgttcagcttcaagtcccagactttcctcctctttggataataaagaaaaggagaaagatagtaaaggaactaggcttgcatatgttgcacctacaataccaagacgactagccagtacatctgacattgaagagaaagaaaacagagattcttcaagtttgcgaacaagtagttcatatacaaggagaaaatgggaagatgatcttaaaaaaaatagctcagttaatgaaggatcaacgtatcataaaagttgctcctttggtagaagacaagatgatttgattagttctagtgttccaagcaccacatcaacaccaacagttacctctgcagctgggcttcagaaaagcctgctttccagcacaagcactactacaaagattacaacgggttcttcctcagcaggcacacaaagcagtacctcaaatcgtttgtgggctgaggatagtactgagaaagaaaaggacagtgttcctacggcagtgaccattcctgttgctccaactgttgtaaatgctgcagcttctaccacaaccctgactacaactactgctggcactgtctcctccacaacagaggtcagggagagacgcagatcatacctcactcctgttagggatgaagagtctgaatcccaaagaaaagcaagatctagacaagcaagacaatctagaagatcaacacagggagtgacattaactgatcttcaagaagctgagaaaacaataggaagaagtcgttctacccgaaccagagaacaagaaaatgaagaaaaagaaaaagaggaaaaagagaaacaagataaagagaaacaagaagaaaagaaggagtcagaaacatctagagaagatgaatataaacaaaagtactccagaacgtatgatgagacttaccagcgttataggccagtatcaacttcaagttcaaccactccatcctcttcactttctactatgagcagttcactgtatgcttcaagtcaactaaacaggccaaatagtcttgtaggcataacttctgcttactccagaggaataacaaaagaaaatgaaagagagggagaaaaaagagaagaggagaaagaaggagaagataaatcacaacctaaatcaatcagagaacgacgacgaccaagagagaaaagaagatctacaggagtttcattttggacacaagatagtgatgaaaatgaacaagaacaacaatcagacacagaagagggatccaataagaaagaaactcagacggattccatttctagatatgaaaccagttctacatcagctggtgatcgatatgattccttgctgggtcgctctggatcatacagttacttagaagaaagaaaaccttacagcagcaggctagaaaaggatgactcaactgactttaaaaagctttatgaacaaattctagctgaaaatgaaaagctgaaggcacagctacatgatacaaatatggaactaacagatcttaaattacagttggaaaaggccacccagagacaagaaagatttgctgatagatcactgttggaaatggaaaaaagggaacgaagagctctagaaagaagaatatctgaaatggaagaagagctcaaaatgttaccagacctaaaagcagacaaccagaggctaaaggatgaaaatggggccttgatcagagttataagcaaactttccaaataaaaaaaaaaaaagcagcaagtaatggaattgcacatattagtaacccagtggaccataattggcagtcactggaagtctgggaagaatccttggagactgtcattttcggatatcctgccaaatgccctcttatctagaatttttgtttcattttgtttaattttctggggtgtttttgttgttgttggtttgttttttgttttttttttttaatcaagaccattgtttcatgttaatgcagctgctgagaagatttttttttaatgactgagaaaacttgtttacagctccagcatataaggaaagtgttcaaggccagatatgcctcagatatttaaccagtaagccttagttgtacataaatacttttgtgtcaacaaaaactttcagctctcacagaagacagttactcaacattttttgatgtgccacagtttcgagtttttcgatatttaaattttttggcttttcatctaagtttgggtttgtattttttccttctaaactcttcatgtggcagagtcttctatgttttcacagctttttcattacagaaaagaacacttgctcttctgtgattattgtcatgtattaggctaatgctgtgttgtctcccacctggaactgaattgcttggtggaacatatgctttcactgtttgtgcaatatgcatttatttcttatatgaatgctttaaagtcatttgaggttagatcttttaattcctattttctgcttcattggtcactttttttttattgtagtataagatgttagattctgtaatcttcacattcattttagcaggtactgagtgatgctgtatatacaaataagtgtattgttttgatttttagaccaccacatggcatgcttgactatttcttatttcaaatgtctgctaatgcagagtaggctactccatgatagtgttaaaaaacaaaatttgctaacaatgtgatataaagactttaaaagttacacattatgtggagccctatctttacaaaagtttcctactgtaaagtgcttttattttcagttttcacttgatagtactcaaccataattaaagttgcataagataattgctttacatttcacatacctatatttatctgagtgctgtctaaaactgttgtgctagccaaagtaatgctatgaaatcatttgcagaattaacccgtgagttaatgttaaatgcactgttattgccatgtgaagaggcatcgactttgataccaccatcatgttcagaccattttatacatttcagtggcctttttttttttttaaggaaaaaaaagcgcaaaaccaagtacatagtgacgatggcttttatttggacaaatagcttttatattttcattaaaccatgcaaaaaatactacatctttctggcacataactgtctccttaaccactgaacagttcagccatttgaataaattgtacattgtaaagcttatagtagctgattgtattattgattgtattgtatactatattaaatgtgaatttgtaccccttcattttaaaaggtcattgtgttctactgcctattttagattactttggggttggggaagggtgttttggtaagcaggattttaagtcctctctctctcgattggttttatgaagatataaggttatgctcttactaccaagctcaggattcttgattttaaaggtacaaggatagttgtgggagttttatttttggttatatttgaactgtatgaatggtaggtctgaatattgagaatttccatggtcttatgtggaggtaaaatatacagataactgatcagtttgagtgactgcaacatgttctggttgcacaacagttaggcatgctatggtttatatttgtgaagtttggatgcctgtgaagaatgattaaatacatgtttctaatattttagtgttttgtatttaggttatttattctttgtatctaaaactgaacagctactgtgctatattgattttattggtagtattgagcagaccttgtttctgcatgaaactagttgcaaaacccactattttggaaataaaaatgtattttgacatatacaaataaaatgaataaaactattttaaatgtgtgaacttttaatgaagacattttaaattttgttctgagatatttaaattctgcatgggtattttttcactaattgctttacttggataccaatttgaataatttctgaaattgtaatattgtgataatttgcacagcattgtacatgcacatctgtaaatgcaatcttaattgccatatttatgcaatctgtgtaccaatttggacaaatgtttatatattttacataaagctgtctgtatgcagaatctga

SEQ ID NO: 48 - protein, 1030 aa, Homo sapiens
MKMADAKQKRNEQLKRWIGSETDLEPPVVKRQKTKVKFDDGAVFLAACSSGDTDEVLKLLHRGADINYANVDGLTALHQACIDDNVDMVKFLVENGANINQPDNEGWIPLHAAASCGYLDIAEFLIGQGAHVGAVNSEGDTPLDIAEEEAMEELLQNEVNRQGVDIEAARKEEERIMLRDARQWLNSGHINDVRHAKSGGTALHVAAAKGYTEVLKLLIQAGYDVNIKDYDGWTPLHAAAHWGKEEACRILVDNLCDMEMVNKVGQTAFDVADEDILGYLEELQKKQNLLHSEKRDKKSPLIESTANMDNNQSQKTFKNKETLIIEPEKNASRIESLEQEKVDEEEEGKKDESSCSSEEDEEDDSESEAETDKTKPLASVTNANTSSTQAAPVAVTTPTVSSGQATPTSPIKKFPTTATKISPKEEERKDESPATWRLGLRKTGSYGALAEITASKEGQKEKDTAGVTRSASSPRLSSSLDNKEKEKDSKGTRLAYVAPTIPRRLASTSDIEEKENRDSSSLRTSSSYTRRKWEDDLKKNSSVNEGSTYHKSCSFGRRQDDLISSSVPSTTSTPTVTSAAGLQKSLLSSTSTTTKITTGSSSAGTQSSTSNRLWAEDSTEKEKDSVPTAVTIPVAPTVVNAAASTTTLTTTTAGTVSSTTEVRERRRSYLTPVRDEESESQRKARSRQARQSRRSTQGVTLTDLQEAEKTIGRSRSTRTREQENEEKEKEEKEKQDKEKQEEKKESETSREDEYKQKYSRTYDETYQRYRPVSTSSSTTPSSSLSTMSSSLYASSQLNRPNSLVGITSAYSRGITKENEREGEKREEEKEGEDKSQPKSIRERRRPREKRRSTGVSFWTQDSDENEQEQQSDTEEGSNKKETQTDSISRYETSSTSAGDRYDSLLGRSGSYSYLEERKPYSSRLEKDDSTDFKKLYEQILAENEKLKAQLHDTNMELTDLKLQLEKATQRQERFADRSLLEMEKRERRALERRISEMEEELKMLPDLKADNQRLKDENGALIRVISKLSK

SEQ ID NO: 49 - DNA, 1579 bp, Homo sapiens
agcccacagcagtccgtgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaatgaaaaaaaaaaaaa

SEQ ID NO: 50 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 51 - DNA, 4093 bp, Homo sapiens
gtgagagagtgagcgagacagaaagagagagaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctcaaaaaaaaa

SEQ ID NO: 52 - protein, 969 aa, Homo sapiens
MAEDDPYLGRPEQMFHLDPSLTHTIFNPEVFQPQMALPTADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 53 - DNA, 1777 bp, Homo sapiens
ctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcagctgctgtggattggattatgccatttggaataagaatgctgttaagagcacacaagccagggagaagtgaaatgacaacctcactagatacagttgagacctttggtaccacatcctactatgatgacgtgggcctgctctgtgaaaaagctgataccagagcactgatggcccagtttgtgcccccgctgtactccctggtgttcactgtgggcctcttgggcaatgtggtggtggtgatgatcctcataaaatacaggaggctccgaattatgaccaacatctacctgctcaacctggccatttcggacctgctcttcctcgtcacccttccattctggatccactatgtcagggggcataactgggtttttggccatggcatgtgtaagctcctctcagggttttatcacacaggcttgtacagcgagatctttttcataatcctgctgacaatcgacaggtacctggccattgtccatgctgtgtttgcccttcgagcccggactgtcacttttggtgtcatcaccagcatcgtcacctggggcctggcagtgctagcagctcttcctgaatttatcttctatgagactgaagagttgtttgaagagactctttgcagtgctctttacccagaggatacagtatatagctggaggcatttccacactctgagaatgaccatcttctgtctcgttctccctctgctcgttatggccatctgctacacaggaatcatcaaaacgctgctgaggtgccccagtaaaaaaaagtacaaggccatccggctcatttttgtcatcatggcggtgtttttcattttctggacaccctacaatgtggctatccttctctcttcctatcaatccatcttatttggaaatgactgtgagcggagcaagcatctggacctggtcatgctggtgacagaggtgatcgcctactcccactgctgcatgaacccggtgatctacgcctttgttggagagaggttccggaagtacctgcgccacttcttccacaggcacttgctcatgcacctgggcagatacatcccattccttcctagtgagaagctggaaagaaccagctctgtctctccatccacagcagagccggaactctctattgtgttttaggtcagatgcagaaaattgcctaaagaggaaggaccaaggagatgaagcaaacacattaagccttccacactcacctctaaaacagtccttcaaacttccagtgcaacactgaagctcttgaagacactgaaatatacacacagcagtagcagtagatgcatgtaccctaaggtcattaccacaggccaggggctgggcagcgtactcatcatcaaccctaaaaagcagagctttgcttctctctctaaaatgagttacctacattttaatgcacctgaatgttagatagttactatatgccgctacaaaaaggtaaaactttttatattttatacattaacttcagccagctattgatataaataaaacattttcacacaatacaataagttaactattttattttctaatgtgcctagttctttccctgcttaatgaaaagcttgttttttcagtgtgaataaataatcgtaagcaaca

SEQ ID NO: 54 - protein, 373 aa, Homo sapiens
MPFGIRMLLRAHKPGRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 55 - DNA, 3960 bp, Homo sapiens
gtttttctctgcttggtgagcagagataaagggggcagcaggaccgggcccaccagccatccgggctgcccacgcaaaccacagggccgaatccggagccgcccaaggccacacagctaagccgagtgcgtgaatgcttatgtgaccgtgtgaaggaggttcccaccgtgtggctgtgggggatggaaaaaggctacttggaaagatgtagaagaccttcgagtaaacagttacgtttcagaaacagagcctgctcagaatgtgtacttggtgggattctattcttagggacgcttctttcttctgagagacccgagctctgtggcgagtggcacaggcagggccccttcctttcctagttgggttctgacagctccgaggcagtggtttacacaaccaacacgaaacatttctacgatccacccgattcctcccctcattgatattcaggaagcagctctccttcccctgccttcagctcaagtttgctgagcttttgtttcatttgtgaatacttcttgctggaagtccctcacccagagaccagtgctcccaacggcagagcagcgggggagataaagaactggtgacacgtggctgtacattcagcacagctgtggtgtccccaagtgccatgacccaggagccattcagagaggagctggcctatgaccggatgcccacgctggagcggggccggcaagaccccgccagctatgccccagacgcgaagccgagcgacctgcagctgtcgaagagactgcccccctgcttcagccacaagacgtgggtcttctctgtgctgatggggagctgcctcctggtgacctcggggttttcgctgtacctggggaacgtgttcccggctgagatggattacttgcgctgtgctgcaggctcttgcatcccctcggcaattgtgagcttcaccgtctccaggaggaacgccaatgtgattcccaactttcagatattgtttgtttccacgtttgctgtgaccactacgtgtttaatttggtttggatgcaaactagtcctgaacccatcagcaataaacatcaacttcaacctcatcctgctgctcctgctggagctgctcatggcggccacggtgatcatcgctgcacggtccagcgaggaggactgcaagaaaaagaagggctccatgtctgacagcgccaacattctggacgaagtgccatttcctgctcgggtcctgaaatcttactcagtcgtcgaggtaatcgcaggcatctctgccgtcctcggggggatcattgccctgaacgtggatgactcagtttcaggcccacacctctcagtgacgttcttttggatcctagtggcctgctttccaagtgccattgccagtcatgtggcagcagagtgtcccagcaagtgtctggtggaggtcctgattgccataagcagcctcacgtctccgctgctgttcacagcctctggatatctgtcattcagcatcatgagaatcgtggagatgtttaaggattacccgccagccataaaaccatcctacgatgtgctgctgctgctgctgctgctagtgctcctgctgcaggccggcctcaacacgggcaccgccatccagtgcgtgcgcttcaaggtcagtgcaaggctgcagggtgcatcctgggacacccagaacggcccgcaggagcgcctggctggggaggtggccaggagccccctgaaggagttcgacaaggagaaagcctggagagccgtcgtggtgcaaatggcccagtgacccccagacgcggaaaccgggtggcagcgcccagcctggccccaagcatggaaacgcacaacccctaatcgccctgagctactgcttctaacacctcttttcccttgtgtgagggcaaaccaggctgcaggtggggttttcacttcctagggtagtttaattttaaaataggccaatgttggctagtctgtgcctcagtgagatcagtcagctccgagtggctcccgtgtcgtaacagcaggagcatggccgcaacttcccaggccgaggaagggcccccggctcggcctcttgagagccccacccctgaactggccccagctcctcttcctgcctctctcatggcttgggctggagtgggctctctggacctgaccagactgtgggtccctgcgtctcctgcccactctgaccgggcttcctccctccacgcttagggtctgtcccgggtactcagtcagcccagtgggatcttacccacttccctgcaaggtgcacctgccccaggctcaggctgcccagcggctcttcctggacagtgagagcagggctgggcgcctctgtcctggcccgggagccgcaggggcccctcctccagagcctgggcgcaagcgacacaggctgccgctgctctcccaggtgaaatccacaccagtccacgccgggtcgcctgccctgtctccctacttagacccagtcattctagagggatccaccgccacactggccggcccacgtcctgggtgctgtcatgcccagcttggagtgccacgtggccgctgcccacgtcccgggcactgtcatgcccagcttggagtgccacatggccgctgcccacgtcccgggcactgtcatgcccagcttggagtgccacgtggccgctgctgtgacaggcagtgttcttgggggtggggctgcatccaaggctttgtaaaccggctggaccacgtctccctggccccagtgaccgggggaagctgagcccctccctcctgtgtttgctcccattactcaaaatgcaggacagatcaggtcagagcccaggaattctcacaggttcacccagcgccctctacctcctagcaagtactttgtcttgatcctcactgagaaggccccagggcagcggtcttctccatctccgctgttttggggtcttagggtacagcccaggcggtcactgcccacctgccaggctgcagggacagttgggtgtgagaataacactggctttgggtagtgccatggccaggagtgggtttccctgcgtctcctcgtcccgagggcgcctgggtcctcccagctgacggcagtaaatccacagtgagttggggcgactgtgaaactggaatgctgttactttgataattactttccagcaggtgttttccttcacaatggttttgtttctttccttctgatctgagaagacatgaacgttttctcttcaccgccgtggggtgtattgactggtcccccatgggctgctggaaaggcccggagatgcatctgtggcctggggccatcaagatcaaagaaccaggaggcctgggagatgcagctggatggggcggcctgcagaccctgccagggggtttgaggaccctcccaggtttcccactgcggaacaggagtgactctggctgccaagataccttcatggtgttcatgacaagtggaatcattattttcaaccattgaagggggatgcaggcaagacaccttcccagctgctcctagaggggacaagccaggccctctctgcagtcctcggcagctccggaaggacacagtcaggggccgggcaaacactttggccacagccccaaacaagcgccaccgtgggagaggagaggctgctgtcactggtaccggatgcagaccccaccctgtctgcaggccacccccacctccctgcagctttgaggctggcggggtctgctcctgggaatggggtgggagccacagggacgacccggggcgggctgatgtcttcttgggggcagaccagagagctcaagtttcagagtcagaattaggcacttggagcgtttttgctggcttgcactttcttattttcttattttagagcgcttaaaaaatccggaaaaatggggtttaaaagaactgtctctttcagtctacatttttgtttaatacgcttgagcaataaacgctgacttgcagacgtg

SEQ ID NO: 56 - protein, 377 aa, Homo sapiens
MTQEPFREELAYDRMPTLERGRQDPASYAPDAKPSDLQLSKRLPPCFSHKTWVFSVLMGSCLLVTSGFSLYLGNVFPAEMDYLRCAAGSCIPSAIVSFTVSRRNANVIPNFQILFVSTFAVTTTCLIWFGCKLVLNPSAININFNLILLLLLELLMAATVIIAARSSEEDCKKKKGSMSDSANILDEVPFPARVLKSYSVVEVIAGISAVLGGIIALNVDDSVSGPHLSVTFFWILVACFPSAIASHVAAECPSKCLVEVLIAISSLTSPLLFTASGYLSFSIMRIVEMFKDYPPAIKPSYDVLLLLLLLVLLLQAGLNTGTAIQCVRFKVSARLQGASWDTQNGPQERLAGEVARSPLKEFDKEKAWRAVVVQMAQ

SEQ ID NO: 57 - DNA, 1926 bp, Homo sapiens
gtggatgagctgtgagtgcgcgcgcgtgcgcggggccgcgacctgtgccggctcgagcccgctgggcactcggaggcgcgcacgtcgttccccgccctcccgccgccgcccgccctcgctctctcgcgctaccctcccgccgcccgcggtcctccgtcggttctctcgttagtccacggtctggtcttcagctacccgccttcgtctccgagtttgcgactcgcggaccggcgtccccggcgcgaagaggctggactcggattcgttgcctgagcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcattttttaaattcccgttttgtcactttttctgataaaagatggccatattaccccttttcggccccatgtatctcagtaccccatggagctgggctaagtaaataggaattggtttcacgcctgaggcaattagacactttggaagatggcataacctgtctcacctggacttaagcatctggctctaattcacagtgctcttttctcctcactgtatccaggttccctcccagaggagccaccagttctcatgggtggcactcagtctctcttctctccagctgactaaactttttttctgtaccagttaatttttccaactactaatagaataaaggcagttttctaaaaaaa

SEQ ID NO: 58 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 59 - DNA, 22 bp, Artificial Sequence
  Description of Artificial Sequence Synthetic primer
cctgaaggaactgaaaggaaag

SEQ ID NO: 60 - DNA, 20 bp, Artificial Sequence
  Description of Artificial Sequence Synthetic primer
ttggcagatatgcagggagt

SEQ ID NO: 61 - DNA, 22 bp, Artificial Sequence
  Description of Artificial Sequence Synthetic primer
ccctccactgtaacgaagactc

SEQ ID NO: 62 - DNA, 21 bp, Artificial Sequence
  Description of Artificial Sequence Synthetic primer
cacacccagtagcagtcatcc

SEQ ID NO: 63 - DNA, 25 bp, Artificial Sequence
  Description of Artificial Sequence Synthetic primer
gaacactgagctaaaaactctcctg

SEQ ID NO: 64 - DNA, 19 bp, Artificial Sequence
  Description of Artificial Sequence Synthetic primer
gagaccatggcactgaacg

SEQ ID NO: 65 - DNA, 18 bp, Artificial Sequence
  Description of Artificial Sequence Synthetic primer
gggctgtcacggagatca

SEQ ID NO: 66 - DNA, 20 bp, Artificial Sequence
  Description of Artificial Sequence Synthetic primer
ccatgatggtcacattctgc

SEQ ID NO: 67 - DNA, 25 bp, Artificial Sequence
  Description of Artificial Sequence Synthetic primer
catccgtaaagacctctatgccaac

SEQ ID NO: 68 - DNA, 20 bp, Artificial Sequence
  Description of Artificial Sequence Synthetic primer
accagaggcatacagggaca